(19) United States
(12) Patent Application Publication
(10) Pub. No.: US 2015/0217046 A1
(43) Pub. Date: Aug. 6, 2015

(54) DOSING REGIMENS FOR SUBCUTANEOUSLY INFUSIBLE ACIDIC COMPOSITIONS

(71) Applicant: SynAgile Corporation, Wilson, WY (US)

(72) Inventors: Adam Heller, Austin, TX (US); Ephraim Heller, Wilson, WY (US)

(21) Appl. No.: 14/405,633

(22) PCT Filed: Jun. 4, 2013
(86) PCT No.: PCT/US13/44049

§ 371 Date: Dec. 4, 2014

Related U.S. Application Data
(60) Provisional application No. 61/771,489, filed on Mar. 1, 2013.
(60) Provisional application No. 61/657,108, filed on Jun. 8, 2012.
(60) Provisional application No. 61/655,730, filed on Jun. 5, 2012.

(51) Int. Cl. A61M 5/142; A61K 31/165; A61K 31/198; A61K 38/47; A61K 31/325

(52) U.S. Cl. CPC A61M 5/14248; A61K 38/47; A61K 31/325; A61K 31/198; A61K 31/165; C12Y 302/01035

(57) ABSTRACT

The invention features methods, compositions, dosing regimens, and infusion pumps for subcutaneously infusing acidic solutions of L-DOPA prodrugs, such as esters and amides of L-DOPA, for the treatment of Parkinson's disease. The methods and acidic compositions of the invention can reduce the severity and rate of occurrence of transient local swelling, erythema, and persistent subcutaneous granulomas associated with subcutaneous delivery of certain agents used in the treatment of Parkinson's disease.

### CROSS-REFERENCE TO RELATED APPLICATIONS

This application claims benefit of U.S. Provisional Ser. No. 61/655,730, filed Jun. 5, 2012, U.S. Provisional Ser. No. 61/657,108, filed Jun. 8, 2012, and U.S. Provisional Ser. No. 61/771,489, filed Mar. 1, 2013, each of which is incorporated herein by reference in its entirety.

### BACKGROUND OF THE INVENTION

The invention relates to compositions, including levodopa esters, for the treatment of Parkinson's disease.

Parkinson's disease (PD) is characterized by the inability of the dopaminergic neurons in the substantia nigra to produce the neurotransmitter dopamine. PD impairs motor skills, cognitive processes, autonomic functions and sleep. Motor symptoms include tremor, rigidity, slow movement (bradykinesia), and loss of the ability to initiate movement (akinesia) (collectively, the “off” state). Non-motor symptoms of PD include dementia, dysphagia (difficulty swallowing), slurred speech, orthostatic hypotension, seborrheic dermatitis, urinary incontinence, constipation, mood alterations, sexual dysfunction, and sleep issues (e.g., daytime somnolence, insomnia).

After more than 40 years of clinical use levodopa therapy remains the most effective method for managing PD and provides the greatest improvement in motor function. Consequently, levodopa (LD) administration is the primary treatment for PD. Levodopa is usually orally administered. The orally administered levodopa enters the blood and part of the levodopa in the blood crosses the blood brain barrier. It is metabolized, in part, in the brain to dopamine which temporarily diminishes the motor symptoms of PD. As the neurodegeration underlying PD progresses, the patients require increasing doses of levodopa and the fluctuations of brain dopamine levels increase. When too much levodopa is transported to the brain, dyskinesia sets in, (uncontrolled movements such as writhing, twitching and shaking); when too little is transported, the patient re-enters the off state. As PD progresses, the therapeutic window for oral formulations of levodopa narrows, and it becomes increasingly difficult to control PD motor symptoms without inducing motor complications. In addition, most PD patients develop response fluctuations to oral levodopa therapy, such as end of dose wearing off, sudden on/off's, delayed time to on, and response failures.

Besides levodopa, other drugs commonly used for treatment of PD include DDC inhibitors, such as carbidopa and benserazide; dopamine receptor agonists, such as pramipexole, ropinirole, bromocriptine, pergolide, piribedil, cabergoline, Lisuride, and apomorphine; MAO-B inhibitors, such as rasagiline and selegiline; COMT inhibitors, such as entacapone and tolcapone; anticholinergics, such as trihexiphenidyl, benztropine, biperiden, and ethopropazine; and amantadine.

Most of the oral levodopa is metabolized before reaching the brain. Peripheral levodopa metabolization to dopamine causes nausea, tremors, and stiffness. Nausea is reduced and bioavailability in the brain is increased by co-administration of DDC-inhibitors, primarily CD or benserazide. CD extends the plasma half-life of levodopa to approximately 90 minutes. These DDC-inhibitors do not substantially cross the blood-brain barrier and thus inhibit only peripheral DDC. The results are reduction in side effects caused by dopamine on the periphery and increase of the concentration of levodopa and dopamine in the brain.

Standard levodopa treatment with oral delivery typically leads to intermittent plasma levodopa levels, which are thought to contribute to motor complications. By contrast, more continuous delivery of levodopa that provides smooth, predictable plasma levels leads to a good therapeutic response with reduced motor complications.

The development of an effective controlled release oral dosage form of levodopa that provides substantially reduced variability in plasma levodopa concentrations and more stable, continuous levodopa delivery to the brain is difficult. Some of the underlying causes of this difficulty, and of the response fluctuations themselves, are believed to be: (a) the short biological half-life of levodopa; (b) erratic gastric emptying, due to effects of PD on the autonomic nervous system; (c) poor absorption of levodopa in the gut in the presence of food, due to competition between levodopa and other amino acids for transport across the intestines; (d) absorption of levodopa taking place only in the duodenum, a short segment of the intestines; and (e) competition between levodopa and other amino acids for active transport from the blood into the brain.

Numerous studies demonstrate that IV infusion of levodopa stabilizes its concentration in plasma and dramatically reduces motor complications and fluctuations (see, for example, Shoulson et al., Neurology 25:1144 (1975); Rosin et al., Arch Neurol. 36:32 (1979); Quinn et al., Lancet. 2:412 (1982); Quinn et al., Neurology. 34:1131 (1984); Nutt et al., N Engl J Med. 310:483 (1984); Hardie et al., Br J Clin Pharmac. 22:429 (1986); and Hardie et al., Brain. 107:487 (1984)).

Likewise, many studies show similarly favorable results upon continuous levodopa infusion directly into the duodenum, using an ambulatory infusion pump (Duodopa therapy). Studies of Duodopa therapy confirm >50% reductions in time spent in the “off” state and time spent with severe dyskinesias. These studies also demonstrate significant improvement in quality of life of the patients (see, for example, Bredberg et al., Eur J Clin Pharmacol. 45:117 (1993); Kurth et al., Neurology 43:1698 (1993); Nilsson et al., Acta Neurol Scand. 97:175 (1998); Syed et al., Mov Disord. 13:336 (1998); Nilsson et al., Acta Neurol Scand. 104:343 (2001); Nyholm et al., Clin Neuropharmacol. 26:156 (2003); Nyholm et al., Neurology. 65:1506 (2005); and Nyholm et al., Clin Neuropharmacol. 31:63 (2008); Antonini et al., Mov Disord. 22:1145 (2007)).

Chronic subcutaneous infusion of drugs such as insulin and pain medications is widely practiced. Such systems are safe for chronic use by patients outside the hospital, convenient, and relatively low cost. It would be desirable to be able to also deliver levodopa or a levodopa prodrug subcutaneously.

The clinically most widely practiced subcutaneous drug infusion is that of insulin in diabetic people. In the management of diabetes less than 5 mg of the drug is infused daily. In the management of Parkinson's disease, apomorphine is being subcutaneously infused in daily doses of 3-30 mg. Most apomorphine-infused patients experience infusion site reactions, such as subcutaneous nodules, indurations, erythemas, tenderness and panniculitis. Advanced Parkinson's disease patients can require a daily L-DOPA dose of 1 g or more, two orders of magnitude greater than the typical daily subcutaneously infused amount of insulin or apomorphine. Another drug, Hizentra (immune globulin), when subcutaneously infused in gram quantities, causes high rates of infusion site reactions. Consequently, the subcutaneous infusion of gram quantities of L-DOPA with an acceptable small rate of incidence of skin reactions requires novel compositions, methods and systems of infusion. Such compositions, methods and systems are disclosed in this invention.

The practicality of subcutaneous levodopa infusion depends on the liquid volume that must be infused for the typical daily dose of 0.3-3 g of levodopa. The subcutaneous infusion of large volumes can cause persistent swelling and edema.

Levodopa is poorly soluble in aqueous solutions near neutral pH. For example, at 25° C. and at pH 7 or pH 5 the solubility of levodopa is about 5 g per liter or less. A patient requiring 1 g levodopa per day would correspondingly require the daily infusion of more than 0.2 liters of the pH 5 or pH 7 solutions. In early studies of IV (intravenous) levodopa infusion, volumes of over 2 L of solution (saline or dextrose and water) per day with less than 1 mg/mL of levodopa were often administered making this administration not only cumbersome, but increasing the risk of thrombophlebitis; to reduce this risk, central venous access was often required and utilized.

The two most widely tested levodopa prodrugs are its methyl ester, known as Melevodopa or LDME, and its ethyl ester, known as Etilevodopa or LDEE (see, for example, Stocchi et al., Mov Disord 25:1881 (2010); Stocchi et al., Clin Neuropharmacol 33:198 (2010); Djaldetti et al., Clin Neuropharmacol 26:322 (2003); and Blindauer et al., Arch Neurol 63:210 (2006)). LDME and LDEE can be unstable in solution, making them difficult to store. Furthermore, their subcutaneous infusion can cause infusion site reactions exemplified by transient local swelling, erythema, and persistent subcutaneous granulomas.

The invention features stable compositions and dosing regimens that can permit subcutaneous infusion of levodopa, or a levodopa prodrug, for the treatment of Parkinson's disease while reducing the severity and rate of occurrence of subcutaneous infusion site reactions.

### ABBREVIATIONS AND DEFINITIONS

The term “CD” refers to Carbidopa.

The term “carbidopa prodrug” refers to carbidopa esters, carbidopa amides, and salts thereof, such as the hydrochloride salt of carbidopa ethyl ester, carbidopa methyl ester, or carbidopa amide.

The term “COMT” refers to catechol-O-methyl transferase.

The term “DDC” refers to DOPA decarboxylase.

The term “hyaluronic acid” refers to hyaluronic acid and salts thereof.

The term “extracellular matrix degrading enzyme” means an enzyme that can break down extracellular matrix at the site of infusion, resulting in improved tissue permeability for an LD-prodrug infused at the site. Extracellular matrix degrading enzymes include enzymes catalyzing the hydrolysis of hyaluronic acid (hyaluronan), a glycosaminoglycan, chondroitin, or collagen, such as a hyaluronidase, glycosaminoglycanase, collagenase (e.g. cathepsin), serine proteases, thiol proteases, and matrix metalloproteases, of which the human enzymes are preferred and the recombinant human enzymes are most preferred. Examples of such enzymes which can be used in the methods and compositions of the invention are described in U.S. Pat. Nos. 4,258,134; 4,820,516; 7,871,607; 7,767,429; 7,829,081; 7,846,431; 7,871,607; 8,187,855; and 8,105,586, and U.S. Patent Publication Nos. 20090304665; 20110053247; 20120101325; and 20110008309, each of which is incorporated by reference. Human hyaluronidases which can be used in the methods and compositions of the invention are also described, for example, in U.S. Pat. Nos. 3,945,889; 6,057,110; 5,958,750; 5,854,046; 5,827,721; and 5,747,027, each of which is incorporated herein by reference. Commercially available hyaluronidases which can be used in the methods and compositions of the invention include Hydase (PrimaPharm Inc.), Vitrase® (ISTA Pharmaceuticals), Amphadase® (Amphastar Pharmaceuticals), and Hylenex® (sold by Halozyme Therapeutics).

The term “IV” refers to intravenous.

The term “LD” refers to levodopa, also known as L-DOPA, or a salt thereof.

The term “LD₅₀” refers to the median lethal oral dose of an LD prodrug in rats at 48 hours (e.g., the dose of LD prodrug required to kill half the rats within 48 hours after ingestion of the LD prodrug).

The term “LDE” refers to an LD prodrug that is a levodopa ester of formula (I):

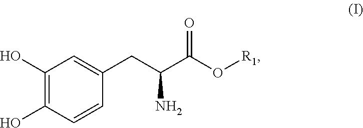

or a pharmaceutically acceptable salt thereof. In formula (I), R₁ is selected from C₁₋₆ alkyl, C₂₋₆ alkenyl, C₂₋₆ alkynyl, C₂₋₆ heterocyclyl, C₆₋₁₂ aryl, C₇₋₁₄ alkaryl, C₃₋₁₀ alkheterocyclyl, and C₁₋₇ heteroalkyl. In particular preferred embodiments, OR₁ is OCH₃, OCH₂CH₃, OCH₂CH₂CH₃, OCH(CH₃)₂, OCH₂CH₂CH₂CH₃, OCH(CH₃)CH₂CH₃,O-benzyl, O-cyclohexyl, OCH₂CH₂OH, OCH₂CH(CH₃)OH, an LD ester of sorbitol, an LD ester of mannitol, an LD ester of xylitol, or an LD ester of glycerol. LDEs are hydrolyzed in vivo to form LD and an alcohol. The LDEs of the invention and their hydrolysis products have an LD₅₀ in rats of greater than 3 millimoles/kg. The subcutaneously infused LDE can be, for example, the addition salt of the shown base with hydrochloric acid, LDEE.HCl. In the acidic solutions of the invention (I) is typically a cation, where the primary amine is an ammonium ion.

The term “LDA” refers to an LD prodrug that is a levodopa amide of formula (III):

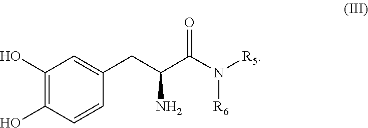

In formula (III), each of R₅ and R₆ is, independently, selected from H, C₁₋₆ alkyl, C₂₋₆ alkenyl, C₂₋₆ alkynyl, C₂₋₆ heterocyclyl, C₆₋₁₂ aryl, C₇₋₁₄ alkaryl, C₃₋₁₀ alkheterocyclyl, and C₁₋₇ heteroalkyl. In particular preferred embodiments, R₅ is H or CH₃, and R₆ is CH₃, CH₂CH₃, CH₂CH₂CH₃, benzyl, 2-deoxy-2-glucosyl, or CH₂CH₂NH₂. LDAs are hydrolyzed in vivo to form LD and an amine or ammonium salt. The LDAs of the invention and their hydrolysis products have an LD₅₀ in rats of greater than 3 millimoles/kg. The LDA can be stored, for example, in its free base form and is typically infused as an acid addition salt. The subcutaneously infused LDA can be, for example, the addition salt of the shown base with hydrochloric acid. In the acidic solutions of the invention (III) is typically a cation, where the primary amine is an ammonium ion.

The term LDEE refers to levodopa ethyl ester, or a salt thereof. In acidic solutions it is mostly protonated LDEE, i.e. it is a cation. The formula of its free base is (I) where R₁═CH₂CH₃. LDEE can be stored as the free base. It is typically infused as LDEE.HCl, the addition salt with hydrochloric acid.

The term “LDME” refers to levodopa methyl ester, or a salt thereof. The formula of its free base is (I) where R₁═CH₃. LDME can be stored as the free base. It is typically infused as LDME.HCl, the addition salt with hydrochloric acid.

The term “LD prodrug” refers to a pharmaceutical composition suitable for subcutaneous infusion. It forms LD upon its hydrolysis. The LD prodrug suitable for infusion is the acid addition salt of a free base of either Formula I or Formula III, such as an acid addition salt with hydrochloric acid. Examples include LDA, LDE, LDEE, and LDME, and their salts. The salts can be formed by neutralizing the amine of the free base with an acid, such as HCl.

The term “MAO-B” refers to monoamine oxidase-B.

As used herein, “neutral amino acid” refers to an amino acid having only one carboxylic acid and only one amine function. Although phenolic amino acids like LD and OMD are partly ionized to anions and hydrated protons at neutral pH, they are classified as neutral.

The term “PD” refers to Parkinson's disease.

As used herein, the term “pH” refers to the pH measured using a pH meter having a glass electrode connected to an electronic meter.

The term “polybasic acid” means an acid having two or more ionizable functions and acid salts of these acids. Examples of polybasic acids include citric acid, succinic acid, pyrophosphoric acid and phosphoric acid and examples of their acid salts include monosodium citrate, disodium citrate, monosodium, disodium succinate and monosodium phosphate. For the acidic solutions of this invention, the infused compositions are administered at a pH that can include polybasic acid anions where only one of the acidic functions is ionized and/or can include polybasic acids that are not ionized.

The term “s.c.” refers to subcutaneous. Subcutaneous means in or below the skin. It can be, for example, intradermal, in the subcutis, in connective tissue or intramuscular. The s.c. infusion can be, for example, at a depth between about 1 mm and about 17 mm below the epidermis, e.g., between about 3 mm and about 10 mm below the epidermis.

The term “administration” or “administering” refers to any route for giving a dosage of LD or LD prodrug (e.g., LDA or LDE) to a subject, including oral, pulmonary, and parenteral routes of administration. Typically administration will include subcutaneous infusion of LD or LD prodrug to a subject. The dosage form of the invention preferably includes subcutaneous infusion, optionally using an infusion pump.

As used herein, “aqueous” refers to formulations of the invention including greater than 10%, 20%, 35%, 50% or 80% (w/w) water.

As used herein, the term “cannula” refers to a tube that can be inserted into the body (e.g., for the delivery of a pharmaceutical composition of the invention). The cannula can be formed from an organic polymer, such as in plastic tubing, or a metallic hollow needle, or other designs known in the art.

As used herein, “co-infused” refers to two or more pharmaceutically active agents, formulated together, or separately, and infused simultaneously, either to the same site (e.g., infused via the same cannula), or adjacent sites (e.g., infused via separate cannulas within 1 cm of each other).

As used herein “continuous infusion” refers to uninterrupted infusion for a period of at least 4 hour. Typical daily durations of continuous infusion typically exceed 12 hours, and are usually 16 hours or 24 hours. The rate of infusion may be reduced during intended sleep periods, optionally to nil.

As used herein “intermittent infusion” refers to infusion that is not continuous for at least 4 hours. In the case of frequent intermittent infusion, the frequency is typically at least once every two hours.

As used herein, “infused” or “infusion” includes infusion under the epidermis, typically at a depth between about 1 mm and 17 mm, often into a part of the skin such as fat, dermis, subcutaneous tissue or connective tissue.

As used herein, the term “shelf life” means the shelf life of the inventive LD prodrug product sold for use by consumers, during which period the product is suitable for use by a subject. The shelf life of the LD prodrugs of the invention can be greater than 3, 6, 12, 18, or preferably 24 months. The shelf life may be achieved when the product is stored frozen (e.g., at about −18° C.), stored refrigerated (at about 5±3° C., for example at about 4±2° C.), or stored at room temperature (e.g., at about 25° C.). The LD prodrug product sold to consumers may be the pharmaceutical composition ready for infusion, or it may be its components. For example, the LD prodrug product for use by consumers may be the dry solid LD prodrug and, optionally, the solution used for its reconstitution; or the LD prodrug stored in an acidic solution and, optionally, a neutralizing basic solution.

As used herein, the term “operational life” means the time period during which the aqueous pharmaceutical composition containing the LD prodrug is suitable for infusion into a subject, under actual product usage conditions. The operational life of the LD prodrugs of the invention can be greater than 12 hours, 24 hours, 48 hours, 72 hours, 96 hours (4 days), or 7 days. It typically requires that the product is not frozen or refrigerated. The product is often infused at room temperature (e.g., about 25° C.), at body temperature (about 37° C.), or in-between (e.g., 30° C.).

As used herein, the term “pulsed dosing regimen” refers to a method for infusing an LD-prodrug including two or more delivery periods during which the LD-prodrug solution is infused to a subject at a site of infusion for a period of from 1 to 800 seconds, 1 second to 1 hour, 1 second to 2 hours, or 1 second to 3 hours separated by non-delivery periods during which the time averaged rate at which the LD-prodrug solution is infused to the subject at the site is substantially reduced. In the pulsed dosing regimens of the invention the non-delivery period can be shorter or longer than the delivery period. For example, the ratio of the length of the delivery period to the length of the non-delivery period can be 4:1 to 1:4.

As used herein, the term “substantially reduced” refers to the reduction in the time averaged rate at which the LD-prodrug solution is infused to the subject at the site during the non-delivery period for the pulsed dosing regimens of the invention. When the time averaged rate at which the LD-prodrug solution is infused to the subject at the site during the non-delivery period less is than 20%, 10%, 8%, 5%, 4%, 2%, 1%, or 0% of the time averaged rate at which the LD-prodrug solution is infused to the subject at a site during the delivery period, the reduced rate of infusion is “substantially reduced.”

As used herein, the term “split dose regimen” refers to a method for infusing an LD prodrug including three or more subcutaneous infusions of the LD prodrug at three sites separated by at least 1 cm. For example, the split dose infusion can be performed using a trifurcated cannula wherein at any given time two arms of the cannula positioned at first and second sites are in a delivery period mode and actively infusing, while one arm of the cannula positioned at a third site is in a non-delivery mode (e.g., inactive and not infusing). The infusion system can be programmed to cycle through delivery period and non-delivery period modes to deliver a pulsed dosing regimen.

As used herein, “stable” refers to formulations of the invention which are “oxidatively stable” and “hydrolytically stable.” Stable formulations exhibit a reduced susceptibility to chemical transformation (e.g., oxidation and/or hydrolysis) prior to infusion into a subject. Stable dry or liquid formulations are those having a shelf life during which less than 10%, 5%, 4%, 3%, 2% or less than 1% of the LD prodrug (e.g., LDA or LDE) is oxidized or hydrolyzed when stored for a period of 3, 6, 12, 18, or 24 months. In general, the solutions of the stable formulations remain clear and are colorless or lightly yellow colored, not darkly colored, meaning that they have no substantial visible precipitate and are not substantially oxidized, after their storage. Stable liquid formulations have an operational life during which less than 10%, 5%, 4%, 3%, 2% or less than 1% of the LD prodrug (e.g., LDA or LDE) is oxidized or hydrolyzed over a period of 8 hours, 12 hours, 16 hours, 24 hours, 48 hours, 72 hours, 96 hours, or 7 days. An “oxidatively stable” formulation exhibits a reduced susceptibility to oxidation during its shelf life and/or its operational life, during which less than 10%, 5%, 4%, 3%, or less than 2% of the LD prodrug (e.g., LDA or LDE) is oxidized. A “hydrolytically stable” formulation exhibits a reduced susceptibility to hydrolysis during its shelf life and/or operational life in which less than 20%, 10%, 5%, 4%, 3%, 2% or less than 1% of the LD prodrug (e.g., LDA or LDE) is hydrolyzed.

As used herein, “substantially free of LD precipitate” refers to formulations of the invention that are clear and without visible precipitates of LD.

As used herein, “substantially free of oxygen” refers to compositions of the invention packaged in a container for storage or for use wherein the packaged compositions are largely free of oxygen gas (e.g., less than 10%, or less than 5%, of the gas that is in contact with the composition is oxygen gas) or wherein the partial pressure of the oxygen is less than 15 torr, 10 torr, or 5 torr. This can be accomplished by, for example, replacing a part or all of the ambient air in the container with an inert atmosphere, such as nitrogen, carbon dioxide, argon, or neon, or by packaging the composition in a container under a vacuum.

As used herein, “substantially free of water” refers to compositions of the invention packaged in a container (e.g., a cartridge) for storage or for use wherein the packaged compositions are largely free of water (e.g., less than 2%, 1%, 0.5%, 0.1%, 0.05%, or less than 0.01% (w/w) of the composition is water). This can be accomplished by, for example, drying the constituents of the formulation prior to sealing the container.

As used herein, the term “treating” refers to infusing a pharmaceutical composition for prophylactic and/or therapeutic purposes. To “prevent disease” refers to prophylactic treatment of a subject who is not yet ill, but who is susceptible to, or otherwise at risk of, a particular disease. To “treat disease” or use for “therapeutic treatment” refers to infusing treatment to a subject already suffering from a disease to ameliorate the disease and improve the subject's condition. The term “treating” also includes treating a subject to delay progression of a disease or its symptoms. Thus, in the claims and embodiments, treating is the infusion to a subject either for therapeutic or prophylactic purposes.

As used herein, the terms “alkyl” and the prefix “alk-” are inclusive of both straight chain and branched chain groups and of cyclic groups, i.e., cycloalkyl. Cyclic groups can be monocyclic or polycyclic and preferably have from 3 to 6 ring carbon atoms, inclusive. Exemplary cyclic groups include cyclopropyl, cyclobutyl, cyclopentyl, and cyclohexyl groups.

By “C₁₋₆ alkyl” is meant a branched or unbranched hydrocarbon group having from 1 to 6 carbon atoms. A C₁₋₆ alkyl may be substituted or unsubstituted, may optionally include monocyclic or polycyclic rings. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₁₋₆ alkyls include, without limitation, methyl, ethyl, n-propyl, isopropyl, cyclopropyl, cyclopropylmethyl, n-butyl, iso-butyl, sec-butyl, tert-butyl, and cyclobutyl. The basic functions such as amino functions can be protonated, i.e. acid addition salts.

By “C₂₋₆ alkenyl” is meant a branched or unbranched hydrocarbon group containing one or more double bonds and having from 2 to 6 carbon atoms. A C₂₋₆ alkenyl may be substituted or unsubstituted, may optionally include monocyclic or polycyclic rings. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₂₋₁₂ alkenyls include, without limitation, vinyl, allyl, 2-cyclopropyl-1-ethenyl, 1-propenyl, 1-butenyl, 2-butenyl, 3-butenyl, 2-methyl-1-propenyl, and 2-methyl-2-propenyl. The basic functions such as nitrogen comprising functions can be protonated, i.e. acid addition salts.

By “C₂₋₆ alkynyl” is meant a branched or unbranched hydrocarbon group containing one or more triple bonds and having from 2 to 12 carbon atoms. A C₂₋₆ alkynyl may be substituted or unsubstituted, may optionally include monocyclic or polycyclic rings. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. C₂₋₆ alkynyls include, without limitation, ethynyl, 1-propynyl, 2-propynyl, 1-butynyl, 2-butynyl, and 3-butynyl. The basic functions such as amino functions can be protonated, i.e. acid addition salts.

By “C₆₋₁₂ aryl” is meant an aromatic group having a ring system comprised of carbon atoms with conjugated π electrons (e.g., phenyl). The aryl group has from 6 to 12 carbon atoms. Aryl groups may optionally include monocyclic, bicyclic, or tricyclic rings, in which each ring desirably has five or six members. The aryl group may be substituted or unsubstituted. Exemplary substituents include alkyl, hydroxy, alkoxy, aryloxy, sulfhydryl, alkylthio, arylthio, halide, fluoroalkyl, carboxyl, hydroxyalkyl, carboxyalkyl, amino, aminoalkyl, monosubstituted amino, disubstituted amino, and quaternary amino groups. The amino functions can be protonated, i.e. acid addition salts.

By “C₇₋₁₄ alkaryl” is meant an alkyl or heteroalkyl substituted by an aryl group (e.g., benzyl, phenethyl, phenoxyethyl, or 3,4-dichlorophenethyl) having from 7 to 14 carbon atoms.

By “C₁₋₇ heteroalkyl” is meant a branched or unbranched alkyl, alkenyl, or alkynyl group having from 1 to 7 carbon atoms in addition to 1, 2, 3 or 4 heteroatoms independently selected from the group consisting of N, O, S, and P. Heteroalkyls include, without limitation, saccharide radicals, tertiary amines, secondary amines, ethers, thioethers, amides, thioamides, carbamates, thiocarbamates, hydrazones, imines, phosphodiesters, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, hydroxyalkyl, carboxyalkyl, and carboxyl groups. The basic, e.g., nitrogen comprising functions can be protonated, i.e., acid addition salts. Examples of C₁₋₇ heteroalkyls include, without limitation, methoxymethyl and ethoxyethyl.

By “C₂₋₆ heterocyclyl” is meant a stable 5- to 7-membered monocyclic or 7- to 14-membered bicyclic heterocyclic ring which is saturated partially unsaturated or unsaturated (aromatic), and which consists of 2 to 6 carbon atoms and 1, 2, 3 or 4 heteroatoms independently selected from N, O, and S and including any bicyclic group in which any of the above-defined heterocyclic rings is fused to a benzene ring. The heterocyclyl group may be substituted or unsubstituted and can be protonated, i.e., an acid addition salt. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. The nitrogen and sulfur heteroatoms may optionally be oxidized. The heterocyclic ring may be covalently attached via any heteroatom or carbon atom which results in a stable structure, e.g., an imidazolinyl ring may be linked at either of the ring-carbon atom positions or at the nitrogen atom. A nitrogen atom in the heterocycle may optionally be quaternized. Preferably when the total number of S and O atoms in the heterocycle exceeds 1, then these heteroatoms are not adjacent to one another. Heterocycles include, without limitation, saccharide radicals.

By “C₃₋₁₀ alkheterocyclyl” is meant an alkyl or heteroalkyl substituted heterocyclic group having from 3 to 10 carbon atoms in addition to one or more heteroatoms (e.g., 3-furanylmethyl, 2-furanylmethyl, 3-tetrahydrofuranylmethyl, or 2-tetrahydrofuranylmethyl). The C₃₋₁₀ alkheterocyclyl can include basic moieties (e.g., when the heteroatom is nitrogen) which are optionally protonated (i.e., as an acid addition salt).

### SUMMARY OF THE INVENTION

The invention features aqueous compositions, methods of subcutaneous infusion and devices for the management of PD. Specifically, it features subcutaneously infusible acidic compositions, subcutaneous infusion methods and devices for maintaining plasma LD concentrations in a desired therapeutic range, thereby reducing the motor symptoms, non-motor symptoms, and response fluctuations associated with PD. Gram quantities of L-DOPA prodrugs are infused into patients to manage the symptoms of Parkinson's disease. LD prodrugs such as LDEE.HCl can be subcutaneously infused, for example, at a rate greater than 20 mg/hr, or more than 30 mg/hr, or more than 40 mg/hr, 50 mg/hr, 60 mg/hr, 70 mg/hr, 80 mg/hr, or 100 mg/hr. Subcutaneous infusion of drugs, including LDEE.HCl at such high mass-rates can lead to adverse local effects, such as nodules, indurations, erythemas, tenderness and panniculitis. The inventor of this disclosure has surprisingly discovered that the incidence of adverse local effects can be reduced by making the infused pharmaceutical composition acidic with a pH between 2.1-3.9, for example pH 2.4±0.3, 2.6±0.3, 2.8±0.3, 3.0±0.3, 3.2±0.3, 3.4±0.3 or 3.6±0.3. The acidic pharmaceutical composition of the LD-prodrug, of a pH between about 2.1 and 3.9, for example between 2.1 and 3.0, or between 3.0 and 3.9, can be subcutaneously infused at a flow rate that is between 0.1 mL per hour per infused site and 2.5 mL per hour per infused site, e.g., between 0.25 mL per hour per infused site and 1.0 mL per hour per infused site. When the pH of the LD prodrug composition is between 2.4 and 3.9, for example between 2.4 and 3.0, or between 3.1 and 3.9, the composition can be subcutaneously infused at an infused site at a flow rate that can exceed 0.3 mL/hr, without causing pain or symptoms like local inflammation, nodule formation, induration, tenderness or swelling.

This invention features a method for treating Parkinson's disease in a subject by subcutaneously administering into the subject a LD prodrug solution in a pulsed dosing regimen, wherein the pulsed dosing regimen includes (i) a delivery period during which the LD prodrug solution is infused at a first site for from 1 second to 3 hours (e.g., 1-10, 10-100, 100-200, 200-400, or 400-800 seconds, 10 minutes to 30 minutes, 30 minutes to 1 hour, 1 hour to 2 hours, or 2 hours to 3 hours); and (ii) following step (i), a non-delivery period during which the LD prodrug solution is administered at a substantially reduced rate to the first site for from 10 to 120 minutes (e.g., 10-20, 20-30, 30-40, 40-50, 50-60, 60-80, 80-100, or 100-120 minutes), and repeating steps (i) and (ii). For example, the delivery period can be repeated at least twice, three times, four times, or six times over an 8 hour period (e.g., an infusion) or, in another example, at least four, six, eight or twelve times over a 16 hour period

In a related aspect, the invention features a method for treating Parkinson's disease in a subject by subcutaneously administering into the subject a LD prodrug solution in a pulsed dosing regimen, wherein the pulsed dosing regimen includes (i) a delivery period during which the LD prodrug solution is infused at a first site for from 1 second to 3 hours (e.g., 1-10, 10-100, 100-200, 200-400, 400-800 seconds, 10 minutes to 30 minutes, 30 minutes to 1 hour, 1 hour to 2 hours, or 2 hours to 3 hours) and (ii) following step (i), a non-delivery period during which the LD prodrug solution is administered at a substantially reduced rate to the first site for from 10 to 120 minutes (e.g., 10-20, 20-30, 30-40, 40-50, 50-60, 60-80, 80-100, or 100-120 minutes); (iii) a delivery period during which the LD prodrug solution is infused at a second site for from 1 second to 3 hours (e.g., 1-10, 10-100, 100-200, 200-400, 400-800 seconds, 10 minutes to 30 minutes, 30 minutes to 1 hour, 1 hour to 2 hours, or 2 hours to 3 hours); and (iv) following step (iii), a non-delivery period during which the LD prodrug solution is administered at a substantially reduced rate to the second site for from 10 to 120 minutes (e.g., 10-20, 20-30, 30-40, 40-50, 50-60, 60-80, 80-100, or 100-120 minutes), and optionally repeating steps and repeating steps (i), (ii), (iii), and (iv). The pulsed dosing regimen can further include (v) a delivery period during which the LD prodrug solution is infused at a third site for from 1 second to 3 hours (e.g., 1-10, 10-100, 100-200, 200-400, 400-800 seconds, 10 minutes to 30 minutes, 30 minutes to 1 hour, 1 hour to 2 hours, or 2 hours to 3 hours) and (vi) following step (v), a non-delivery period during which the LD prodrug solution is administered at a substantially reduced rate to the third site for from 10 to 120 minutes (e.g., 10-20, 20-30, 30-40, 40-50, 50-60, 60-80, 80-100, or 100-120 minutes), and optionally repeating steps (v) and (vi). In some embodiments, the pulsed dosing regimen further includes (vii) a delivery period during which the LD prodrug solution is infused at a fourth site for from 1 second to 3 hours (e.g., 1-10, 10-100, 100-200, 200-400, 400-800 seconds, 10 minutes to 30 minutes, 30 minutes to 1 hour, 1 hour to 2 hours, or 2 hours to 3 hours) and (viii) following step (vii), a non-delivery period during which the LD prodrug solution is administered at a substantially reduced rate to the fourth site for from 10 to 120 minutes (e.g., 10-20, 20-30, 30-40, 40-50, 50-60, 60-80, 80-100, or 100-120 minutes), and optionally repeating steps (vii) and (viii). In still other embodiments, the pulsed dosing regimen further includes (ix) a delivery period during which the LD prodrug solution is infused at a fifth site for from 1 second to 3 hours (e.g., 1-10, 10-100, 100-200, 200-400, 400-800 seconds, 10 minutes to 30 minutes, 30 minutes to 1 hour, 1 hour to 2 hours, or 2 hours to 3 hours) and (x) following step (ix), a non-delivery period during which the LD prodrug solution is administered at a substantially reduced rate to the fifth site for from 10 to 120 minutes (e.g., 10-20, 20-30, 30-40, 40-50, 50-60, 60-80, 80-100, or 100-120 minutes), and optionally repeating steps (ix) and (x). In one particular embodiment, the delivery period is repeated at least twice, three times, four times, or six times over an 8 hour period (e.g., an infusion). In still other embodiments, the delivery period is repeated every 60 to 120 minutes (e.g., 60, 60-80, 80-100, or 100-120 minutes) over an 8 hour period. The pulsed dosing regimen can include administration of the LD prodrug solution to a plurality of sites sequentially, wherein each of the sites are separated from each other by at least 1 cm, 3 cm, or 5 cm (e.g., from 1 to 6 cm, from 3 to 8 cm, or from 4 to 10 cm). In particular embodiments, an extracellular matrix degrading enzyme (e.g., a hyaluronidase, or any extracellular matrix degrading enzyme described herein) is administered at each of the sites (e.g., prior to administration of the dopamine agonist and/or during the non-delivery period). In particular embodiments, the extracellular matrix degrading enzyme is co-infused with the dopamine agonist. In certain embodiments, the dopamine agonist solution is an LD prodrug solution (e.g., a solution containing LDEE or a salt thereof). In certain embodiments, the LD prodrug solution is a solution containing LDEE or a salt thereof. In one particular embodiment, during the non-delivery period (i) no LD prodrug solution is administered; and (ii) for at least a portion of the non-delivery period an aqueous rinse solution is administered to the subject to disperse the LD prodrug from the site of infusion. The aqueous rinse solution can be a saline solution, optionally having a pH of from 4 to 8, and optionally including a venous vasodilator or an extracellular matrix degrading enzyme (e.g., a hyaluronidase, or any extracellular matrix degrading enzyme described herein). In particular embodiments, the aqueous rinse is administered for a period of 1 second to 3 hours (e.g., 1-10, 10-100, 100-200, 200-400, or 400-800 seconds), or from 10 to 120 minutes (e.g., 10-20, 20-30, 30-40, 40-50, 50-60, 60-80, 80-100, or 100-120 minutes). The aqueous rinse can be administered to each site following each delivery period.

In any of the above methods the time averaged rate at which the LD prodrug is administered during the non-delivery period is less than 10%, 8%, 6%, 4%, 2%, or 1% of the time averaged rate at which the LD prodrug is infused during the delivery period. In certain embodiments, the time averaged rate at which the LD prodrug is administered during the non-delivery period is from 0 μmol/minute to 0.25 μmol/minute (e.g., 0 to 0.10, 0 to 0.05, or 0 to 0.25 μmol/minute, preferably 0 μmol/minute) or from 0.25 μmol/minute to 0.75 μmol/minute (e.g., 0.25 to 0.50, 0.35 to 0.55, or 0.60 to 0.75 μmol/minute, preferably 0 μmol/minute). The non-delivery period can be at least twice as long as the delivery period (e.g., at least 2.5×, 3×, 4×, or 5× the length of the delivery period). Alternatively, the ratio of the length of the delivery period to the non-delivery period can be from 1:4 to 4:1, preferably from 1:1 to 4:1. In certain embodiments, the non-delivery period is from 10 to 90 min, for example from 30 to 90 minutes. In one particular embodiment, the LD prodrug solution includes LDE or LDEE. The method can produce (a) a circulating plasma LD concentration greater than 400 ng/mL and less than 7,500 ng/mL (e.g., less than 5,000 ng/mL, 2,500 ng/mL, or 2,000 ng/mL) which is continuously maintained in the subject for a period of at least 8 hours during the pulsed dosing regimen. In particular embodiments, at least ¼, ½, or ¾ of the total daily molar dosage of the LD prodrug and of LD is by subcutaneous infusion of the LD-prodrug. The LD prodrug solution can be subcutaneously infused at such a rate that the circulating LD plasma concentration varies by less than +/−20%, +/−15%, or +/−10% from its mean for a period of at least 1 hour, 2 hours, 4 hours, or 8 hours.

In particular embodiments, the sum of the LD prodrug administered over all sites over a 24 hour period is less than 15 millimoles (e.g., 0.2 to 1, 0.5 to 5, 3 to 7, or 6 to 15 millimoles) and the sum of infusion volume administered over all sites over a 24 hour period is less than 40 mL, 35 mL, 30 mL, 25 mL, 20 mL, 15 mL, or 10 mL. For example, the sum of the LD prodrug administered over all sites over a 24 hour period can be from 1 to 15 millimoles (e.g., 1 and 3 millimoles, 3 and 6 millimoles, or 6 and 10, or 10 and 15 millimoles) and the sum of infusion volume administered over all sites over a 24 hour period can be between 3 and 40 mL (e.g., 3 and 6 mL, 5 and 16 mL, 10 and 16 mL, 16 and 25 mL, or 25 and 40 mL) over the 24 hour period. For example, the sum of the LD prodrug administered over all sites over a 24 hour period can be (i) less than 15 millimoles and the sum of infusion volume administered over all sites over a 24 hour period can be less than 40 mL; (ii) less than 10 millimoles and the sum of infusion volume administered over all sites over a 24 hour period can be less than 40 mL; or (iii) can be from 1.0 and 15 millimoles and the sum of infusion volume administered over all sites over a 24 hour period can be between 3 and 40 mL over a 24 hour period.

In a preferred embodiment, a LD prodrug, such as LDEE or LDME, is infused at least once every 60-120 minutes over a period of at least 8 hours. The LD prodrug can be infused in an amount sufficient to maintain a circulating plasma LD concentration greater than 400 ng/mL (e.g., greater than 400, 800, 1200, or 1600) and less than 7,500 ng/mL (e.g., less than 5,000 ng/mL, 2,500 ng/mL, or 2,000 ng/mL), which is continuously maintained in the subject for a period of at least 8 hours. Preferably, each of the infusion sites is separate from each of the other infusion sites by a distance of greater than 1 cm (e.g., from 1 to 6 cm, from 1 to 3 cm, from 2 to 4 cm, or from 3 to 6 cm).

In a related aspect, the invention features a method for treating Parkinson's disease in a subject by subcutaneously infusing into the subject a LD prodrug solution in a split dose regimen, wherein the split dose regimen includes (i) infusing a first dose of the LD prodrug solution to a first site; and (ii) following step (i), (ii) infusing a second dose of the LD prodrug solution to a second site; and (iii) following step (ii), infusing a third dose of the LD prodrug solution to a third site, wherein the first site, the second site, and the third site are separated from each other by at least 1 cm, 3 cm, or 5 cm (e.g., from 1 to 6 cm, from 1 to 3 cm, from 2 to 4 cm, or from 3 to 6 cm), and wherein the first dose, the second dose, and the third dose are infused within 2 hours, 1 hour, 30 minutes, 10 minutes, 5 minutes, or 3 minutes of each other. In particular embodiments, an extracellular matrix degrading enzyme (e.g., a hyaluronidase, or any extracellular matrix degrading enzyme described herein) is administered by injection or infusion at each of the sites (e.g., prior to administration of the LD prodrug solution). In particular embodiments, the extracellular matrix degrading enzyme is co-infused with the LD prodrug solution. These infusion devices can deliver a fixed dose or a dose that can be adjusted by the patient or the patient's caregiver. In certain embodiments, the LD prodrug solution is a solution containing LDEE or a salt thereof. These infusion devices may include a container or drug reservoir that is prefilled with LD prodrug or a container or drug reservoir that may be filled by the patient or the patient's caregiver. The invention includes methods to split the infusion dose among multiple infusion sites to minimize infusion site reactions.

The invention further features a method for treating Parkinson's disease in a subject by subcutaneously administering into the subject an LDEE solution in an amount sufficient to treat the Parkinson's disease, wherein the LDEE solution has a pH of 3.7±0.3 (e.g., 3.7±0.2 or 3.7±0.1) or a pH of 3.5±0.3 (e.g., 3.5±0.2 or 3.5±0.1) or a pH of 3.2±0.3 (e.g., 3.2±0.2 or 3.2±0.1) or a pH 2.9±0.3 (e.g., 2.9±0.2 or 2.9±0.1) and includes from 0.15 M to 4.0 M LDEE, or a salt thereof. In certain embodiments, the LDEE solution includes from 0.15 M to 1.6 M, from 0.15 M to 0.75 M, from 0.15 M to 0.5 M, or from 0.15 M to 0.35 M LDEE, or a salt thereof.

In particular embodiments, the LDEE solution remains substantially free of precipitated LD solids for at least 12 months when stored at about 5±3° C. (e.g., about 4° C.). In still other embodiments, the LDEE solution remains substantially free of precipitated solid LD for at least 48 hours when stored at about 25° C. In yet other embodiments, the LDEE solution remains substantially free of precipitated solid LD for at least 8 hours, e.g., for 16 hours, or for 24 hours or for 48 hours when stored at about 37° C. In particular embodiments, the LDEE solution remains substantially free of precipitated solid LD when thawed after being stored frozen (e.g., at about −18° C. or at about −3° C.) for at least 3 months, 6 months, 12 months, 18 months, or 24 months. In a related embodiment the LDEE solution is a ready-to-administer solution which is stored and administered (i.e., without raising the pH and without diluting with water). The LDEE solution can have a shelf life of greater than 3, 6, 12, 18, or preferably 24 months; and an operational life of greater than 12 hours, 24 hours, 48 hours, 72 hours, 96 hours (4 days), or 7 days. The LDEE solution can be administered by infusion (e.g., subcutaneously infused into the subject via one or more ambulatory infusion pumps as described herein), administered in a pulsed dosing regimen as described herein.

The invention also features an LDEE solution having a pH of 3.7±0.3 (e.g., 3.7±0.2 or 3.7±0.1) or a pH of 3.5±0.3 (e.g., 3.5±0.2 or 3.5±0.1) or a pH of 3.2±0.3 (e.g., 3.2±0.2 or 3.2±0.1) or a pH 2.9±0.3 (e.g., 2.9±0.2 or 2.9±0.1) and including from 0.15 M to 1.6 M LDEE, or a salt thereof (e.g., 0.15±0.05 M, 0.25±0.05 M, 0.35±0.05 M, 0.45±0.05 M, 0.85±0.25 M, or 1.35±0.25 M LDEE, or a salt thereof). In particular embodiments, the LDEE solution further includes a buffer (e.g., citrate, acetate, or any other suitable buffer described herein). The LDEE solution can be used in any of the dosing regimens described herein.

Convenient sites for subcutaneous administration include the shoulder, upper arm, thigh, and abdomen. In particular embodiments of the above methods, the dopamine agonist solution is administered proximate a large muscle (e.g., the diaphragm, trapezius, deltoid, pectoralis major, triceps brachii, biceps, gluteus maximus, sartorius, biceps femoris, rectus femoris, and gastrocnemius) at a depth between 5 mm and 15 mm below the surface of the skin of the subject, or administered into subcutis or fat at a depth between 2 mm and 10 mm below the dermis of the subject.

In particular embodiments of any of the above methods, the method further includes orally administering an NSAID (e.g., aspirin, salicylic acid, or a salt thereof).

In another embodiment, the LD prodrug solution, when stored, can include greater than 0.15 M LD prodrug (e.g., 0.25±0.1; 0.4±0.1, 0.5±0.1, 0.6±0.1, 0.7±0.1, 0.8±0.2, 1.0±0.3, 1.5±0.5, 2.0±0.5, 0.6±0.3, 0.75±0.25, 1.0±0.5, 1.5±0.5, 2.0±0.5, 2.5±0.5, 3.0±0.5, 3.5±0.5, or greater than 3.5 moles per liter moles per liter) and is substantially free of precipitated solid LD when stored for 24 hours at about 25° C., but is preferably infused at concentrations below 1.6 M. The LD prodrug can be selected from LDAs, LDEs, and salts thereof. In one particular embodiment, the LD prodrug is LDEE, LDME, or a salt thereof. The LD prodrug solution, when stored, can have a pH of from 3.0 to 6.0 (e.g., 3.5 to 5.0, 3.3±0.3, 3.6±0.3, 3.9±0.3, 4.2±0.3, 4.5±0.3, 4.4±0.2, 4.5±0.5 or 5.0±0.5) and includes from 0.15 M to 4.0 M LDEE (e.g., 0.4±0.1, 0.5±0.1, 0.6±0.1, 0.7±0.1, 0.8±0.2, 1.0±0.3, 1.25±0.25, 1.5±0.25, 1.75±0.25, 2.0±0.25, 2.5±0.25, 2.75±0.25, 3.0±0.5, or 3.5±0.5 M LDEE). For example, the LD prodrug solution, when stored, can have a pH of from 2.5 to 4.6 and include from 0.15 M to 1.6 M LDEE or LDME; can have a pH of from 2.5 to 4.6 and include from 0.25 M to 0.75 M LDEE or LDME; can have a pH of from 2.6 to 3.9 and include from 0.25 M to 0.75 M LDEE or LDME. In one particular embodiment, the LD prodrug solution has a pH of from 2.1 to 3.9 and comprises from 0.15 M to 1.6 M LDEE, or a salt thereof. In particular embodiments, the LD prodrug solution includes a buffer, such as citrate, succinate, pyrophosphate, or phosphate buffer. The LD prodrug solution can be subcutaneously infused into the subject via one or more ambulatory infusion pumps, each pump pumping into one or more implanted cannulas, for example into two cannulas, three cannulas, or four cannulas, the cannulas spaced optionally at distances of at least 1 cm, 2 cm, 3 cm, or 4 cm from each other. In particular embodiments, the infusion is via two or more infusion pumps. In still other embodiments, the infusion is via a two-compartment infusion pump.

In one embodiment of any of the above methods, the LD prodrug solution is subcutaneously infused into the subject via a bifurcated, trifurcated, quadrifurcated or other multifurcated infusion set. For pulsed infusions the infusion set or its fluidic connection to the pump can have a valve periodically preventing flow to a particular cannula while there is flow to other cannulas. For example, the split dose infusion can be performed using a multifurcated cannula wherein at any given time one or more arms of the cannula positioned at one or more sites are in a delivery period mode and actively infusing, while one or more arms of the cannula positioned at one or more different sites are in a non-delivery mode (e.g., inactive and not infusing, or infusing at only very low flow rates). The infusion system can be programmed to cycle through delivery period and non-delivery period modes to deliver a pulsed dosing regimen by cycling through the multiple arms of a mutifurcated cannula.

In certain embodiments, the method includes the steps of: (i) providing a solution including greater than 0.15M LD prodrug (e.g., 0.25±0.1; 0.4±0.1, 0.5±0.1, 0.6±0.1, 0.7±0.1, 0.8±0.2, 1.0±0.3, 1.5±0.5, 2.0±0.5, 0.6±0.3, 0.75±0.25, 1.0±0.5, 1.5±0.5, 2.0±0.5, 2.5±0.5, 3.0±0.5, 3.5±0.5, or greater than 3.5 moles per liter moles per liter) and having a pH of 2.7±0.7 (e.g., 2.1±0.3, 2.5±0.3, or 2.7±0.3), wherein less than 10%, 5%, or 3% of the LD prodrug is hydrolyzed when stored at 5±3° C. (e.g., at about 4° C.) for a period of 3 months or longer; (ii) raising the pH of the solution to 3.0 to 6.0 (e.g., 3.0 to 5.0, 3.0±0.3, 3.3±0.3, 3.6±0.3, 3.9±0.3, 4.5±0.3, 4.4±0.2, 4.5±0.5 or 5.0±0.5), adjusted, for example, with a salt of citric acid, pyrophosphoric acid, succinic acid, or phosphoric acid, to form the LD prodrug solution while optionally also diluting the solution with water such that the resulting LD-prodrug concentration is between 0.15 M and 1.6 M, for example between 0.2 M and 0.4 M, 0.4 M and 0.5 M, 0.5 M and 0.6 M, 0.6 M and 0.7 M, 0.7 M and 0.8 M, 0.8 M and 1.0 M, 1.0 M and 1.2 M, 1.2 M and 1.4 M, or 1.4 M and 1.8 M; and (iii) administering at least a portion of the LD prodrug solution into the subject. Step (iii) is optionally performed within 72 hours, 48 hours, or 24 hours of performing step (ii). The LD prodrug solution is optionally co-infused with an extracellular matrix degrading enzyme (e.g., a hyaluronidase). In particular embodiment, the LD prodrug (such as LDEE) is infused at one or more sites (e.g., one, two, three, four, or more sites), wherein the volume infused at a single site is less than 40 mL, 35 mL, 30 mL, or 25 mL (e.g., between 1-5 mL, 2-20 mL, 3-10 mL, 10-25 mL, or 20-40 mL) per 24 hour period; the combined amount of drug delivered at all sites is typically less than 15 millimoles (e.g., between 5-15 millimoles, 0.25-10 millimoles, or 0.4-0.6 millimoles) per 24 hour period; and the pH of the aqueous solution is between 3.0-6.0 (e.g., 3.5-5.3). For example, between 1 and 10, 1 and 5, 1 and 3, 1 and 2, 0.5 and 1, or 0.2 and 0.5 millimoles of LD prodrug can be infused at a single site during a 24 hour period. It has been empirically determined that infusing LDEE under these conditions reduces the incidence of pain, inflammation, swelling, and subcutaneous granuloma formation, while providing adequate operational stability.

In a related embodiment stable, ready-to-administer solution is stored and administered, i.e., without the step of raising the pH and without diluting with water. The LD prodrug concentration of the stored and infused solution can be between 0.15 M and 1.6 M, for example between 0.2 M and 0.3 M; 0.3 M and 0.4 M; 0.4 M and 0.5 M; 0.5 M and 0.6 M; 0.6 M and 0.7 M; 0.7 M and 1.2 M; or 1.2 M and 1.6 M; and its pH can be between about 3.0 and about 4.2, for example its pH can be pH 3.7±0.3 or a pH of 3.5±0.3 (e.g., 3.5±0.2 or 3.5±0.1) or a pH of 3.2±0.3 (e.g., 3.2±0.2 or 3.2±0.1) or a pH 2.9±0.3 (e.g., 2.9±0.2 or 2.9±0.1). The solution has a shelf life of greater than 3, 6, 12, 18, or preferably 24 months; and an operational life of greater than 12 hours, 24 hours, 48 hours, 72 hours, 96 hours (4 days), or 7 days. The typical daily administered volume of the solution is between about 1 mL and about 40 mL, which is optionally subcutaneously infused at one, two, three, four or more sites.

The invention further features a container including a reconstitutable solid or liquid which can be mixed with water to form a ready-to-administer LD prodrug solution having a pH of 3.7±0.3 (e.g., 3.7±0.2 or 3.7±0.1) or a pH of 3.5±0.3 (e.g., 3.5±0.2 or 3.5±0.1) or a pH of 3.2±0.3 (e.g., 3.2±0.2 or 3.2±0.1) or a pH 2.9±0.3 (e.g., 2.9±0.2 or 2.9±0.1). In certain embodiments, the reconstitutable solid or liquid is substantially free of water. In other embodiments, the reconstitutable solid or liquid includes a buffer (e.g., citrate, acetate, or any other suitable buffer described herein). In some embodiments, the reconstitutable solid or liquid includes LDEE, or a salt thereof.

In a related aspect, the invention features a method for treating Parkinson's disease in a subject by (i) reconstituting a reconstitutable solid or liquid with water to form an LDEE solution having a pH of 3.7±0.3 (e.g., 3.7±0.2 or 3.7±0.1) or a pH of 3.5±0.3 (e.g., 3.5±0.2 or 3.5±0.1) or a pH of 3.2±0.3 (e.g., 3.2±0.2 or 3.2±0.1) or a pH 2.9±0.3 (e.g., 2.9±0.2 or 2.9±0.1) and including from 0.15 M to 1.6 M LDEE, or a salt thereof (e.g., 0.15±0.5 M, 0.25±0.5 M, 0.35±0.5 M, 0.45±0.5 M, 0.85±0.25 M, or 1.35±0.25 M LDEE, or a salt thereof); and (ii) subcutaneously infusing the LDEE solution into the subject in an amount sufficient to treat the Parkinson's disease. In certain embodiments, the reconstitutable solid or liquid is substantially free of water. In other embodiments, the reconstitutable solid or liquid includes a buffer (e.g., citrate, acetate, or any other suitable buffer described herein). The LDEE solution can be administered in a pulsed dosing regimen, a split dosing regimen, or any other dosing regimen described herein.

In a related aspect, the invention features an ambulatory infusion pump system for the treatment of Parkinson's disease in a subject including: (i) a drug reservoir including a LD prodrug solution (e.g., an LDE or LDEE solution); (ii) a first cannula in fluid communication with the drug reservoir for subcutaneously administering the LD prodrug solution into the subject at a first site; and (iii) a software unit including a program for controlled infusion of the LD prodrug solution in a pulsed dosing regimen, wherein the pulsed dosing regimen includes (a) a delivery period during which the LD prodrug solution is administered to the first site for from 1 second to 3 hours (e.g., 1-10, 10-100, 100-200, 200-400, or 400-800 seconds, 10 minutes to 30 minutes, 30 minutes to 1 hour, 1 hour to 2 hours, or 2 hours to 3 hours); and (b) following step (a), a non-delivery period during which the LD prodrug solution is administered at a substantially reduced rate to the first site for from 10 to 120 minutes, and optionally repeating steps (a) and (b). In particular embodiments, the ambulatory infusion pump system further includes (iv) a second cannula in fluid communication with the drug reservoir for infusing the LD prodrug solution into the subject at a second site, wherein the pulsed dosing regimen further includes (c) a delivery period during which the LD prodrug solution is administered to the second site for from 1 second to 3 hours (e.g., 1-10, 10-100, 100-200, 200-400, or 400-800 seconds, 10 minutes to 30 minutes, 30 minutes to 1 hour, 1 hour to 2 hours, or 2 hours to 3 hours); and (d) following step (c), a non-delivery period during which the LD prodrug solution is administered at a substantially reduced rate to the second site for from 10 to 120 minutes, and optionally repeating steps (c) and (d). The ambulatory infusion pump system can further include: (v) a third cannula in fluid communication with the drug reservoir for infusing the LD prodrug solution into the subject at a third site, wherein the pulsed dosing regimen further includes (e) a delivery period during which the LD prodrug solution is administered to the third site for from 1 second to 3 hours (e.g., 1-10, 10-100, 100-200, 200-400, or 400-800 seconds, 10 minutes to 30 minutes, 30 minutes to 1 hour, 1 hour to 2 hours, or 2 hours to 3 hours); and (f) following step (e), a non-delivery period during which the LD prodrug solution is administered at a substantially reduced rate to the third site for from 10 to 120 minutes, and optionally repeating steps (e) and (f). In some embodiments, the ambulatory infusion pump system further includes: (vi) a fourth cannula in fluid communication with the drug reservoir for infusing the LD prodrug solution into the subject at a fourth site, wherein the pulsed dosing regimen further includes (g) a delivery period during which the LD prodrug solution is administered to a fourth site for from 1 second to 3 hours (e.g., 1-10, 10-100, 100-200, 200-400, or 400-800 seconds, 10 minutes to 30 minutes, 30 minutes to 1 hour, 1 hour to 2 hours, or 2 hours to 3 hours); and (h) following step (g), a non-delivery period during which the LD prodrug solution is administered at a substantially reduced rate to the fourth site for from 10 to 120 minutes, and optionally repeating steps (g) and (h). In still other embodiments, the ambulatory infusion pump system further includes: (vii) a fifth cannula in fluid communication with the drug reservoir for infusing the LD prodrug solution into the subject at a fifth site, wherein the pulsed dosing regimen further includes (i) a delivery period during which the LD prodrug solution is administered to a fifth site for from 1 second to 3 hours (e.g., 1-10, 10-100, 100-200, 200-400, or 400-800 seconds, 10 minutes to 30 minutes, 30 minutes to 1 hour, 1 hour to 2 hours, or 2 hours to 3 hours); and (j) following step (i), a non-delivery period during which the LD prodrug solution is administered at a substantially reduced rate to the fifth site for from 10 to 120 minutes, and optionally repeating steps (i) and (j).

In any of the ambulatory infusion pump systems above, the ambulatory infusion pump system can be programmed to repeat the delivery period at least twice, three times, four times, six times, or eight times over any 8 hour period (e.g., an infusion). The pulsed dosing regimen can include administration of the LD prodrug solution to a plurality of sites sequentially, wherein each of the sites are separated from each other by at least 1 cm, 3 cm, or 5 cm (e.g., from 2 to 5 cm, from 3 to 8 cm, or from 4 to 10 cm).

In any of the ambulatory infusion pump systems above, the ambulatory infusion pump system can be programmed to repeat the delivery period every 60 to 120 minutes (e.g., 60, 60-80, 80-100, or 100-120 minutes) over an 8 hour period. In one particular embodiment, the ambulatory infusion pump system is programmed to administer no LD prodrug during the non-delivery period. In still other embodiments, the non-delivery period can be at least twice as long as the delivery period (e.g., at least 2.5×, 3×, 4×, or 5× the length of the delivery period).

The ambulatory infusion pump systems of the invention can include an adhered patch bearing a plurality of cannulas positioned at a plurality of sites (e.g., a triangular arrangement of three cannulas, a square arrangement of four cannulas, a pentagonal arrangement of five cannulas, or a hexagonal arrangement of six cannulas. Preferably, the cannulas are separated from each other by at least 1.0 or 2.0 cm.

The ambulatory infusion pump systems and infusion devices of the invention can further include: (x) a first reservoir containing an acidic aqueous solution including from 0.15 M to 4.0 M (e.g., 0.25±0.1; 0.4±0.1, 0.5±0.1, 0.6±0.1, 0.7±0.1, 0.8±0.2, 1.0±0.3, 1.0±0.5, 1.5±0.5, 2.0±0.5, 2.5±0.5, 3.0±0.5, or 3.5±0.5 moles per liter) LDEE, or a salt thereof; (y) a second reservoir containing a basic aqueous solution; and (z) a means for combining and a means for administering the acidic aqueous solution and the basic aqueous solution into a subject (e.g., a cannula and/or needle in fluid communication with the first drug reservoir and the second drug reservoir for combining and administering the acidic aqueous solution and the basic aqueous solution into a subject, optionally with a mixing chamber). In particular embodiments, the first reservoir contains an acidic aqueous solution having a pH of from 1.5 to 3.5 (e.g., 2.7±0.5, 2.5±0.3, or 2.7±0.3), and the second reservoir contains a basic aqueous solution having a pH of greater than 7.0 (e.g., greater than 7.5, 8.0, or 8.5). The acidic aqueous solution can include a pharmaceutical composition described herein. In particular embodiments, the basic aqueous solution includes a pharmaceutically acceptable potassium and/or a sodium salt of a monobasic, dibasic, tribasic or tetrabasic acid, such as a salt of citric acid; acetic acid; pyrophosphoric acid, succinic acid, or phosphoric acid (e.g., trisodium citrate, sodium acetate, tetrasodium pyrophosphate, disodium succinate, or trisodium phosphate). In a related embodiment, stable, ready-to-administer solution is stored and administered, i.e., without the step of raising the pH and without diluting with water. The LD prodrug concentration of the stored and administered solution can be between 0.15 M and 1 M, for example between 0.2 M and 0.3 M, 0.3 M and 0.4 M, 0.4 M and 0.5 M, 0.5 M and 0.6 M, 0.6 M and 0.7 M, 0.7 M and 0.8 M, or 0.8 M and 1.0 M; and its pH can be between about 3.0 and about 4.2, for example its pH can be pH 3.7±0.3 or a pH of 3.5±0.3 (e.g., 3.5±0.2 or 3.5±0.1) or a pH of 3.2±0.3 (e.g., 3.2±0.2 or 3.2±0.1) or a pH 2.9±0.3 (e.g., 2.9±0.2 or 2.9±0.1). The solution has a shelf life of greater than 3, 6, 12, 18, or preferably 24 months; and an operational life of greater than 12 hours, 24 hours, 48 hours, 72 hours, 96 hours (4 days), or 7 days.

Any of the above devices and methods can further include forming a subcutaneously infusible solution by dissolving in 5 minutes or less at about 25° C. solid LDE or LDA and a solid salt of a polybasic acid of an at least tenfold lesser molar amount than the molar amount of the LDE or the LDA stored in a first container; by adding to the solid mixture HCl of a concentration of less than 2 M, 1.5 M, 1M, 0.75M, 0.6 M or 0.5 M stored in a second container, such that the pH of the resulting solution is 5.5±0.5, 5.0±0.5 or 4.5±0.5, and the solution remains clear, i.e., precipitate-free, when kept at about 25° C. for more than 48 hours or longer or at 37° C. for more than 16 hours. Exemplary LDEs include LDEE and LDME. Exemplary polybasic acid salts include trisodium citrate, disodium citrate, trisodium phosphate or disodium phosphate.

The devices of the invention can further include a composition including: (i) a first container including a sterile aqueous solution containing about 0.15 M to 4.0 M (e.g., 0.25±0.1; 0.4±0.1, 0.5±0.1, 0.6±0.1, 0.7±0.1, 0.8±0.2, 1.0±0.3, 1.25±0.25, 1.5±0.25, 1.75±0.25, 2.0±0.25, 2.5±0.25, 2.75±0.25, 3.0±0.5, or 3.5±0.5 M) LDEE hydrochloride salt and having a pH of from 1.5 to 3.5 (e.g., 2.7±0.5, 2.5±0.3, or 2.7±0.3), wherein less than 10%, 5%, or 3% of the LDEE is hydrolyzed when the first container is stored at 5±3° C. (e.g., about 4° C.) for a period of 3 months; and (ii) a second container including a sterile basic compound (e.g., trisodium citrate, sodium acetate, or any other base described herein) either dissolved in solution or as a solid, reconstitutable base, wherein the combined contents of the first container and the second container form a solution suitable for subcutaneous infusion into a subject, having a pH of from 3.0 to 6.0 (e.g., 3.0 to 5.0, 3.0±0.3, 3.3±0.3, 3.6±0.3, 3.9±0.3, 4.5±0.3, 4.4±0.2, 4.5±0.5 or 5.0±0.5), including greater than or equal to about 0.15 M LDEE (e.g., 0.25±0.1, 0.4±0.1, 0.5±0.1, 0.6±0.3, 1.0±0.3, 1.5±0.5, 2±0.5, or 2.5±0.5 M LDEE), and substantially free of LD precipitate. In particular embodiments, the first container remains substantially free of precipitated LD solids for at least 12 months when stored at about 5±3° C. (e.g., about 4° C.). In still other embodiments, the solution suitable for subcutaneous infusion remains substantially free of precipitated solid LD for at least 48 hours when stored at about 25° C. In yet other embodiments, the solution suitable for subcutaneous infusion remains substantially free of precipitated solid LD for at least 8 hours, e.g., for 16 hours, or for 24 hours or for 48 hours when stored at about 37° C. In particular embodiments, the solution suitable for subcutaneous administration infusion remains substantially free of precipitated solid LD when thawed after being stored frozen (e.g., at about −18° C. or at about −3° C.) for at least 3 months, 6 months, 12 months, 18 months, or 24 months. In a related embodiment stable, a ready-to-administer solution is stored and administered, i.e., without the step of raising the pH and without diluting with water. The LD prodrug concentration of the stored and infused solution can be between 0.15 M and 1 M, for example between 0.2 M and 0.3 M, 0.3 M and 0.4 M, 0.4 M and 0.5 M, 0.5 M and 0.6 M, 0.6 M and 0.7 M, 0.7 M and 0.8 M, or 0.8 M and 1.0 M; and its pH can be between about 3.0 and about 4.2, for example its pH can be pH 3.7±0.3 or a pH of 3.5±0.3 (e.g., 3.5±0.2 or 3.5±0.1) or a pH of 3.2±0.3 (e.g., 3.2±0.2 or 3.2±0.1) or a pH 2.9±0.3 (e.g., 2.9±0.2 or 2.9±0.1). The solution has a shelf life of greater than 3, 6, 12, 18, or preferably 24 months; and an operational life of greater than 12 hours, 24 hours, 48 hours, 72 hours, 96 hours (4 days), or 7 days.

The methods of the invention can further include (i) providing a first container including a sterile aqueous solution containing about 0.15 M to 4.0 M LDEE hydrochloride salt (e.g., 0.25±0.1; 0.4±0.1, 0.5±0.1, 0.6±0.1, 0.7±0.1, 0.8±0.2, 1.0±0.3, 1.25±0.25, 1.5±0.25, 1.75±0.25, 2.0±0.25, 2.5±0.25, 2.75±0.25, 3.0±0.5, or 3.5±0.5 M LDEE hydrochloride salt) and having a pH of from 1.5 to 3.5 (e.g., 2.7±0.5, 2.5±0.3, or 2.7±0.3), wherein less than 10%, 5%, or 3% of the LDEE is hydrolyzed when the first container is stored at 5±3° C. (e.g., about 4° C.) for a period of 3 months; (ii) providing a second container including a sterile basic compound (e.g., sodium citrate, or any other base described herein) either dissolved in solution or as a solid, reconstitutable base; (iii) combining the contents of the first container and the second container form a solution suitable for subcutaneous infusion into a subject, having a pH of from 3.0 to 6.0 (e.g., 3.0 to 5.0, 3.0±0.3, 3.3±0.3, 3.6±0.3, 3.9±0.3, 4.5±0.3, 4.4±0.2, 4.5±0.5, or 5.0±0.5), including greater than or equal to about 0.15 M LDEE (e.g., 0.25±0.1; 0.4±0.1, 0.5±0.1, 0.6±0.1, 0.7±0.1, 0.8±0.2, 1.0±0.3, 1.5±0.5, 2±0.5, 1.5±0.5, 2±0.5, or 2.5±0.5 M LDEE), and substantially free of LD precipitate; and (iv) subcutaneously administering (e.g., infusing) into the subject the solution suitable for subcutaneous infusion. In a related embodiment stable, ready-to-administer solution is stored and administered, i.e., without the step of raising the pH and without diluting with water. The LD prodrug concentration of the stored and administered solution can be between 0.15 M and 1 M, for example between 0.2 and 0.3 M, 0.3 M and 0.4 M; 0.4 M and 0.5 M, 0.5 M and 0.6 M, 0.6 M and 0.7 M, 0.7 M and 0.8 M, or 0.8 M and 1.0 M, and its pH can be between about 3.0 and about 4.2, for example its pH can be pH 3.7±0.3 or a pH of 3.5±0.3 (e.g., 3.5±0.2 or 3.5±0.1) or a pH of 3.2±0.3 (e.g., 3.2±0.2 or 3.2±0.1) or a pH 2.9±0.3 (e.g., 2.9±0.2 or 2.9±0.1). The solution has a shelf life of greater than 3, 6, 12, 18, or preferably 24 months; and an operational life of greater than 12 hours, 24 hours, 48 hours, 72 hours, 96 hours (4 days), or 7 days.

Instead of forming the LD prodrug solution by mixing a more acidic aqueous solution and a basic solution, a solution having a pH between 2.6 and 4.2 could be both stored and administered. The LD prodrug solution could be buffered at pH 3.7±0.5 or a pH of 3.5±0.3 (e.g., 3.5±0.2 or 3.5±0.1) or a pH of 3.2±0.3 (e.g., 3.2±0.2 or 3.2±0.1) or a pH 2.9±0.3 (e.g., 2.9±0.2 or 2.9±0.1), for example with sodium citrates. It could be, for example, an LDE solution, such as an LDEE solution, having a concentration of at least 0.2 M, 0.5 M, 1 M, or 1.5 M. It could be stored refrigerated at 5±3° C., for example at about 4±2° C., for at least 3 months and it could also be infused over a period of 16 hours or longer at ambient temperature, for example at 25±3° C., or even at body temperature, near about 37° C.

The methods and devices of the invention can include a pharmaceutical composition including an aqueous liquid containing greater than 0.15 M (e.g., 0.2 to 0.3, 0.3 to 0.6, 0.6 to 1.4, 1.4 to 2.5, 0.6±0.3, 0.75±0.25, 1.0±0.3, 1.0±0.5, 1.5±0.5, 2.0±0.5, 2.5±0.5, 3.0±0.5, 3.5±0.5, or greater than 3.5 moles per liter) LD prodrug, or a salt thereof, wherein less than 10%, 5%, or 3% of the LD prodrug is hydrolyzed when the pharmaceutical composition is stored at 5±3° C. (e.g., about 4° C.) for a period of 3 months. In certain embodiments, the aqueous liquid has a pH of from 1.5 to 3.5 (e.g., 2.7±0.5, 2.5±0.3, or 2.7±0.3). The pharmaceutical composition can further include a pharmaceutically acceptable excipient, such as a crystal growth inhibitor, hyaluronic acid, and/or antioxidants. In particular embodiments, the LD prodrug is a hydrochloride salt. In still other embodiments, the liquid has a viscosity of between 1.2 cP and 2,000 cP (e.g., from 1.2 cP to 2 cP, 1.5 cP to 5 cP, 2.5 cP to 7.5 cP, 5 cP to 10 cP, 1.2 cP to 200 cP, 10 cP to 200 cP, or 200 cP to 2,000 cP). The pharmaceutical composition can be substantially free of oxygen. In particular embodiments, the liquid includes a polycarboxylate (e.g., hyaluronic acid, succinylated gelatin, poly(acrylic acid), poly(methacrylic acid), poly(glutamic acid), poly(aspartic acid), poly(maleic acid), poly(malic acid), or poly(fumaric acid)). In still other embodiments, the LD prodrug is an acid addition salt of hydrochloric acid, sulfuric acid, or phosphoric acid. In certain embodiments the pharmaceutical composition is a liquid that is supersaturated in LD. In particular embodiments, the pharmaceutical composition can remain substantially free of precipitated solid LD for at least 6 months, 12 months, or 24 months when stored at about 5±3° C. (e.g., about 4° C.). In still other embodiments, the pharmaceutical composition can remain substantially free of precipitated solid LD for at least 3 months, 6 months, 12 months, or 18 months when stored at about 25° C. In particular embodiments, the solubility of LD in the pharmaceutical composition is at least 5 g per liter at about 25° C.

The methods of the invention can further include (i) providing a pharmaceutical composition described above; (ii) raising the pH of the pharmaceutical composition to 3.0 to 6.0 (e.g., 3.0 to 5.0, 3.0±0.3, 3.3±0.3, 3.6±0.3, 3.9±0.3, 4.5±0.3, 4.4±0.2, 4.5±0.5 or 5.0±0.5) to form an LD prodrug solution; and (iii) within 48 hours, 24 hours, or 12 hours of performing step (ii), administering at least a portion of the LD prodrug solution into the subject in an amount sufficient to treat Parkinson's disease. Alternatively, the methods of the invention can include preparing an infusible LD prodrug solution including the steps of: (i) providing an aqueous liquid containing greater than 0.15 (e.g., 0.2 to 0.3, 0.3 to 0.6, 0.6 to 1.4, 1.4 to 2.5, 0.6±0.3, 0.75±0.25, 1.0±0.3, 1.0±0.5, 1.5±0.5, 2.0±0.5, 2.5±0.5, 3.0±0.5, 3.5±0.5, or greater than 3.5 moles per liter) LD prodrug and a pH of from 1.5 to 3.5 (e.g., 2.7±0.5, 2.5±0.3, or 2.7±0.3), or a salt thereof, wherein less than 10%, 5%, or 3% of the LD prodrug is hydrolyzed when said pharmaceutical composition is stored at 5±3° C. for a period of 3 months; (ii) raising the pH of the an aqueous liquid to 3.0 to 6.0 (e.g., 3.0 to 5.0, 3.0±0.3, 3.3±0.3, 3.6±0.3, 3.9±0.3, 4.5±0.3, 4.4±0.2, 4.5±0.5 or 5.0±0.5) to form an infusible LD prodrug solution by combining the aqueous liquid with a base in either reconstitutable solid dosage form or in component solution form (e.g., a base including sodium citrate, sodium acetate, or any other base described herein); and (iii) inserting the infusible LD prodrug solution into an infusion pump, wherein the infusible LD prodrug solution remains substantially free of precipitated LD when kept at about 25° C. for at least 24 hours. Alternatively, a ready-to-administer solution is stored and administered, i.e., without the step of raising the pH and without diluting with water. The LD prodrug concentration of the stored and infused solution can be between 0.15 M and 1 M, for example between 0.2 M and 0.3 M; 0.3 M and 0.4 M, 0.4 M and 0.5 M, 0.5 M and 0.6 M, 0.6 M and 0.7 M, 0.7 M and 0.8 M, or 0.8 M and 1.0 M; and its pH can be between about 2.6 and about 4.2, for example its pH can be pH 3.7±0.3 or a pH of 3.5±0.3 (e.g., 3.5±0.2 or 3.5±0.1) or a pH of 3.2±0.3 (e.g., 3.2±0.2 or 3.2±0.1) or a pH 2.9±0.3 (e.g., 2.9±0.2 or 2.9±0.1). The solution has a shelf life of greater than 3, 6, 12, 18, or preferably 24 months; and an operational life of greater than 12 hours, 24 hours, 48 hours, 72 hours, 96 hours (4 days), or 7 days.

The devices of the invention can further include a pharmaceutical composition suitable for infusion into a subject including an aqueous liquid containing greater than 0.15 M (e.g., 0.2 M to 0.3 M, 0.3 to 0.6, 0.6 to 1.4 to 2.5, 0.6±0.3, 0.75±0.25, 1.0±0.3, 1.0±0.5, 1.5±0.5, 2.0±0.5, 2.5±0.5, 3.0±0.5, 3.5±0.5, or greater than 3.5 moles per liter) LD prodrug, or a salt thereof, wherein the pharmaceutical composition remains substantially free of LD precipitate for at least 24 hours, e.g., for 48 hours or for 72 hours, when stored at about 5±3° C., or for at least 8 hours, e.g., for 16 hours or 24 hours or 48 hours when stored at about 37° C. In particular embodiments, the aqueous liquid has a pH of from 3.0 to 6.0 (e.g., 3.0 to 5.0, 3.0±0.3, 3.3±0.3, 3.6±0.3, 3.9±0.3, 4.5±0.3, 4.4±0.2, 4.5±0.5 or 5.0±0.5). The pharmaceutical composition can further include a pharmaceutically acceptable excipient, such as a crystal growth inhibitor, hyaluronic acid, and/or antioxidants. In particular embodiments, the LD prodrug is a hydrochloride salt. In still other embodiments, the liquid has a viscosity of between 1.2 cP and 2,000 cP (e.g., from 1.2 cP to 2 cP, 1.5 cP to 5 cP, 2.5 cP to 7.5 cP, 5 cP to 10 cP, 1.2 cP to 200 cP, 10 cP to 200 cP, or 200 cP to 2,000 cP). The pharmaceutical composition can be substantially free of oxygen. In particular embodiments, the liquid includes a polycarboxylate (e.g., hyaluronic acid, succinylated gelatin, poly(acrylic acid), poly(methacrylic acid), poly(glutamic acid), poly(aspartic acid), poly(maleic acid), poly(malic acid), or poly(fumaric acid)). In still other embodiments, the LD prodrug is an acid addition salt of hydrochloric acid, sulfuric acid, or phosphoric acid. In certain embodiments the pharmaceutical composition is a liquid that is supersaturated in LD. In certain embodiments the pharmaceutical composition can remain substantially free of LD precipitate for at least 12 hours, 24 hours, 48 hours, or 72 hours when stored at about 5±3° C. In some embodiments the pharmaceutical composition can remain substantially free of LD precipitate for at least 8 hours, 16 hours, for example for 24 hours or for 48 hours, when stored at about 37° C. In particular embodiments, the pharmaceutical composition can be substantially free of precipitated solid LD when thawed after being stored frozen (e.g., at about −18° C. or at about −3° C.) for at least 3 months, 6 months, 12 months, 18 months, or 24 months. In still other particular embodiments the solubility of LD in the pharmaceutical composition is at least 5 g per liter at about 25° C.

The devices of the invention can further include a pharmaceutical composition suitable for infusion into a subject including an aqueous liquid containing greater than 0.15±0.5 M, 0.25±0.5 M, 0.35±0.5 M, or 0.45±0.5 M LD prodrug and buffered at a pH between pH 2.6 and pH 4.2 (e.g., pH 3.7±0.3 or a pH of 3.5±0.3 (e.g., 3.5±0.2 or 3.5±0.1) or a pH of 3.2±0.3 (e.g., 3.2±0.2 or 3.2±0.1) or a pH 2.9±0.3 (e.g., 2.9±0.2 or 2.9±0.1)), remaining essentially free of LD precipitate after being stored at 5±3° C. (for example at 4±2° C.) for at least 3 months (for example for at least 4 months or 6 months) and/or for at least 8, 16, 24 or 48 hours at about 37° C. An example of such a composition is a buffered, optionally citrate buffered 2.7 M or greater concentration LDEE.HCl aqueous solution.

The devices of the invention can further include a stable pharmaceutical composition suitable for infusion into a subject, optionally in the jejunum of a subject including greater than 0.3 M (e.g., 0.6±0.3, 0.75±0.25, 1.0±0.3, 1.0±0.5, 1.5±0.5, 2.0±0.5, 2.5±0.5, 3.0±0.5, 3.5±0.5, or greater than 3.5 moles per liter) LD prodrug, or a salt thereof, dissolved in a non-aqueous liquid, wherein the pharmaceutical composition remains substantially free of LD precipitate for at least 24 hours when stored at about 25° C. The non-aqueous liquid can be a lipid (e.g., a triglyceride, a cholesterol ester, sesame oil, castor oil, or cottonseed oil), an alcohol (e.g., ethanol, glycerol or propylene glycol), N-methyl pyrrolidone, or a mixture thereof. The aqueous solution can be, e.g., a solution of glucose, glycerol, poly(ethylene glycol), the weight % of the exemplary glucose or glycerol or poly(ethylene glycol) being greater than 10%, 20%, 30%, 40%, 50%. The pharmaceutical composition can include an antioxidant (e.g., bisulfite, propofol, salicylic acid or salicylic acid salt, a salt of ascorbic acid (such as sodium ascorbate), p-aminophenol, acetamol, a t-butyl ortho-substituted phenol, or any antioxidant described herein). In one embodiment, the pharmaceutical composition can include a fatty acid salt of the LD prodrug. In certain embodiments, the liquid has at about 20° C. a viscosity of between 1.2 cP and 2,000 cP (e.g., from 1.2 cP to 2 cP, 1.5 cP to 5 cP, 2.5 cP to 7.5 cP, 5 cP to 10 cP, 1.2 cP to 200 cP, 10 cP to 100 cP, 50 cP to 500 cP, 250 cP to 750 cP, 500 cP to 1,000 cP, 750 cP to 2,000 cP, or 50 cP to 1,500 cP). In certain embodiments the pharmaceutical composition can remain substantially free of LD precipitate for at least 12 hours, 24 hours, 48 hours, or 72 hours when stored at about 5±3° C. In particular embodiments, the pharmaceutical composition can substantially free of precipitated solid LD when thawed after being stored frozen (e.g., at about −18° C. or at about −3° C.) for at least 3 months, 6 months, 12 months, 18 months, or 24 months. In still other particular embodiments the solubility of LD in the pharmaceutical composition is at least 5 g per liter at about 25° C. In particular embodiments, the pharmaceutical composition can remain substantially free of precipitated solid LD for at least 6 months, 12 months, or 24 months when stored at about 5±3° C. (e.g., about 4° C.). In still other embodiments, the pharmaceutical composition can remain substantially free of precipitated solid LD for at least 3 months, 6 months, 12 months, or 18 months when stored at about 25° C.

The devices of the invention can further include a stable pharmaceutical composition suitable for infusion into a subject including greater than 0.15 M (e.g., 0.25±0.1; 0.5±0.2, 0.6±0.3, 0.75±0.25, 1.0±0.3, 1.0±0.5, 1.5±0.5, 2.0±0.5, 2.5±0.5, 3.0±0.5, 3.5±0.5, or greater than 3.5 moles per liter) LD prodrug, or a salt thereof, dissolved in a liquid carrier including water and a lipid, wherein the pharmaceutical composition remains substantially free of LD precipitate for at least 24 hours when stored at about 25° C. In particular embodiments, the liquid carrier includes an emulsion or liposomes. The pharmaceutical composition can include an antioxidant (e.g., bisulfite, propofol, salicylic acid or salicylic acid salt, a salt of ascorbic acid (such as sodium ascorbate), p-aminophenol, acetamol, a t-butyl ortho-substituted phenol, or any antioxidant described herein). In one embodiment, the pharmaceutical composition can include a fatty acid salt of the LD prodrug. In certain embodiments, the pharmaceutical composition has at about 20° C. a viscosity of between 1.2 cP and 2,000 cP (e.g., from 1.2 cP to 2 cP, 1.5 cP to 5 cP, 2.5 cP to 7.5 cP, 5 cP to 10 cP, 1.2 cP to 200 cP, 10 cP to 100 cP, 50 cP to 500 cP, 250 cP to 750 cP, 500 cP to 1,000 cP, 750 cP to 2,000 cP, or 50 cP to 1,500 cP). In certain embodiments the pharmaceutical composition can remain substantially free of LD precipitate for at least 12 hours, 24 hours, 48 hours, or 72 hours when stored at about 5±3° C. In particular embodiments, the pharmaceutical composition can substantially free of precipitated solid LD when thawed after being stored frozen (e.g., at about −18° C. or at about −3° C.) for at least 3 months, 6 months, 12 months, 18 months, or 24 months. In still other particular embodiments the solubility of LD in the pharmaceutical composition is at least 5 g per liter at about 25° C. In particular embodiments, the pharmaceutical composition can remain substantially free of precipitated solid LD for at least 6 months, 12 months, or 24 months when stored at about 5±3° C. (e.g., about 4° C.). In still other embodiments, the pharmaceutical composition can remain substantially free of precipitated solid LD for at least 3 months, 6 months, 12 months, or 18 months when stored at about 25° C.

The devices of the invention can further include a stable pharmaceutical composition suitable for infusion into a subject, optionally into the stomach or duodenum or jejunum of a subject, including greater than 0.3 M (e.g., 0.6±0.3, 0.75±0.25, 1.0±0.5, 1.5±0.5, 2.0±0.5, 2.5±0.5, 3.0±0.5, 3.5±0.5, or greater than 3.5 moles per liter) LD prodrug fatty acid salt, wherein the pharmaceutical composition is substantially free of LD precipitate for at least 12 hours, 24 hours, or 48 hours when stored at about 25° C. In particular embodiments, the pharmaceutical composition can substantially free of precipitated solid LD when thawed after being stored frozen (e.g., at about −18° C. or at about −3° C.) for at least 3 months, 6 months, 12 months, 18 months, or 24 months. In particular embodiments, the pharmaceutical composition includes greater than 40 weight % (e.g., 40-60%, 50-70%, 60-90%, or 80-98%) (w/w) carboxylate salt of an LD prodrug dissolved in a liquid carrier. The liquid carrier can be a lipid (e.g., a triglyceride, a cholesterol ester, sesame oil, castor oil, or cottonseed oil), an alcohol (e.g., ethanol, glycerol or propylene glycol), N-methyl pyrrolidone, or a mixture thereof. In particular embodiments the liquid carrier further includes an antioxidant. The liquid carrier can include water and a lipid. In particular embodiments, the liquid carrier includes an emulsion or liposomes. In certain embodiments, the pharmaceutical composition has at about 20° C. a viscosity of between 1.2 cP and 2,000 cP (e.g., from 1.2 cP to 2 cP, 1.5 cP to 5 cP, 2.5 cP to 7.5 cP, 5 cP to 10 cP, 1.2 cP to 200 cP, 10 cP to 100 cP, 50 cP to 500 cP, 250 cP to 750 cP, 500 cP to 1,000 cP, 750 cP to 2,000 cP, or 50 cP to 1,500 cP). In certain embodiments the pharmaceutical composition can remain substantially free of LD precipitate for at least 12 hours, 24 hours, 48 hours, or 72 hours when stored at about 25° C. In particular embodiments, the pharmaceutical composition can substantially free of precipitated solid LD when thawed after being stored frozen (e.g., at about −18° C. or at about −3° C.) for at least 3 months, 6 months, 12 months, 18 months, or 24 months. In particular embodiments the solubility of LD in the pharmaceutical composition is at least 5 g per liter at about 25° C. In particular embodiments, the pharmaceutical composition can remain substantially free of precipitated solid LD for at least 6 months, 12 months, or 24 months when stored at about 5±3° C. (e.g., about 4° C.). In still other embodiments, the pharmaceutical composition can remain substantially free of precipitated solid LD for at least 3 months, 6 months, 12 months, or 18 months when stored at about 25° C.

In any of the above pharmaceutical compositions, the LD prodrug can be selected from LDAs, LDEs, and salts thereof. In particular embodiments, the LD prodrug is LDEE, LDME, or a salt thereof, such as LDEE hydrochloride salt.

The devices of the invention can further include a container including a material that is substantially impermeable to oxygen, the container containing a reconstitutable solid including an LD prodrug, or a salt thereof, wherein the container is substantially free of oxygen and wherein the reconstitutable solid, when reconstituted, is suitable for subcutaneous administration infusion. The invention also features a container including a material that is substantially impermeable to oxygen, the container containing liquid including an LD prodrug, or a salt thereof, wherein the container is substantially free of oxygen and wherein the liquid is suitable for subcutaneous infusion. In particular embodiments, the container can further include a pharmaceutically acceptable excipient, such as a viscosity enhancing agent, an anti oxidant, and/or a preservative. For example, the container can further include from 0.5 to 4.0% (w/w) hyaluronic acid, or any other viscosity enhancing agent described herein; and/or the container can further include an antioxidant (e.g., bisulfite, propofol, salicylic acid or salicylic acid salt, a salt of ascorbic acid (such as sodium ascorbate), p-aminophenol, acetamol, a t-butyl ortho-substituted phenol, or any antioxidant described herein).

In particular embodiments, the LD prodrug in the container is an LDE, or a salt thereof, such as an acid addition salt of LDEE (e.g., LDEE hydrochloride). In certain embodiments, the container is designed to hold less than 35 mL, 30 mL, 25 mL, 20 mL, 15 mL, 10 mL, 5 mL, 3 mL of a liquid including from 0.15 M to 4.0 M LD prodrug, or a salt thereof, (e.g., 0.25±0.1; 0.4±0.1, 0.5±0.1, 0.6±0.1, 0.7±0.1, 0.8±0.2, 1.0±0.3, 0.8±0.3, 1.0±0.5, 1.5±0.5, 2.0±0.5, 2.5±0.5, 3.0±0.5, or 3.5±0.5 moles per liter) and having a pH of from 1.5 to 3.5 (e.g., 2.7±0.5, 2.5±0.3, or 2.7±0.3), wherein the container is substantially free of oxygen.

The methods of the invention can further include: (i) dissolving the solid contents of a container of the invention in buffering agent containing water to form an aqueous solution having a pH of from 3.0 to 6.0 (e.g., 3.0 to 5.0, 3.0±0.3, 3.3±0.3, 3.6±0.3, 3.9±0.3, 4.5±0.3, 4.4±0.2, 4.5±0.5 or 5.0±0.5) and an LD prodrug concentration of from 0.15 M to 4.0 M (e.g., 0.25±0.1; 0.4±0.1, 0.5±0.1, 0.6±0.1, 0.7±0.1, 0.8±0.3, 1.0±0.5, 1.5±0.5, 2.0±0.5, 2.5±0.5, 3.0±0.5, or 3.5±0.5 moles per liter); and (ii) administering (e.g., infusing) the aqueous solution into the subject in an amount sufficient to treat Parkinson's disease. The buffered water can include a pharmaceutically acceptable potassium and/or a sodium salt of a monobasic, dibasic, tribasic or tetrabasic acid, such as a salt of citric acid; acetic acid; pyrophosphoric acid, succinic acid, or phosphoric acid (e.g., trisodium citrate, sodium acetate, tetrasodium pyrophosphate, disodium succinate, or trisodium phosphate). In particular embodiments, the LD prodrug is levodopa ethyl ester. In still other embodiments, the solution infused into the subject is substantially free of precipitated solids; has a pH of from 3.0 to 6.0 (e.g., 3.0 to 5.0, 3.0±0.3, 3.3±0.3, 3.6±0.3, 3.9±0.3, 4.5±0.3, 4.4±0.2, 4.5±0.5 or 5.0±0.5), and includes greater than 0.15 M (e.g., 0.25±0.1; 0.5±0.2, 1.0±0.5 or 1.5±0.5, 2.0±0.5, 2.5±0.5, 3.0±0.5, 3.5±0.5, or greater than 3.5 moles per liter) levodopa ethyl ester.

The invention further features compositions and a method for treating Parkinson's disease in a subject by subcutaneously infusing into the subject an acidic pharmaceutical composition comprising an LD prodrug acid addition salt (such as an acid addition salt of LDEE) in an amount sufficient to treat the Parkinson's disease, wherein the pharmaceutical composition has a pH not greater than about 3.9 and not less than about 2.0 (e.g., between 3.5 and 3.8 or between 3.0 and 3.5, or between 2.5 and 3.0, or between 2.4 and 2.8, or between 2.3 and 3.3, or between 2.3 and 2.9), and includes from 0.15 M to 1.6 M LD prodrug acid addition salt. In certain embodiments, the concentration of LD prodrug acid addition salt in the acidic infused pharmaceutical composition is from 0.15 M to 1.6 M, or from 0.15 M to 0.35 M, or from 0.3 M to 0.6 M, or from 0.5 M to 0.9 M, or from 0.8 M to 1.2 M, or from 1.1 M to 1.6 M. The acidic pharmaceutical composition of the LD-prodrug can be subcutaneously infused at a flow rate that is between 0.1 mL per hour per infused site and 2.5 mL per hour per infused site, e.g. between 0.25 mL per hour per infused site and 1.0 mL per hour per infused site. When the pH of the acidic LD prodrug acid addition salt comprising pharmaceutical composition is between 2.4 and 3.9, the composition can be subcutaneously infused at an infused site at a flow rate that can exceed 0.3 mL/hr without causing pain or symptoms like local inflammation, nodule formation, induration, tenderness or swelling.

The invention features a pharmaceutical composition including an aqueous solution containing from 0.15 to 1.6 M LD prodrug acid addition salt and having a pH of from 2.1 to 3.9 (e.g., 2.1 to 3.0, 2.4±0.3, 2.6±0.3, 3.1 to 3.9, 2.8±0.3, 3.1±0.3, 3.4±0.3, or 3.7±0.2), wherein the pharmaceutical composition is subcutaneously infusible. In some embodiments the pharmaceutical composition includes an aqueous solution containing from 0.15 to 0.7 M LD prodrug acid addition salt. Alternatively, the pharmaceutical composition includes an aqueous solution containing from 0.7 to 1.6 M LD prodrug acid addition salt. In particular embodiments the LD prodrug acid addition salt is an acid addition salt of LDEE or LDME. The pharmaceutical composition can further include a buffer (e.g., citric acid, succinic acid, pyrophosphoric acid, phosphoric acid, citrate, succinate, pyrophosphate, or phosphate). The pharmaceutical composition optionally includes a pharmaceutically acceptable excipient (e.g, any pharmaceutically acceptable excipient described herein). In particular embodiments the pharmaceutical composition is substantially free of oxygen. In some embodiments the pharmaceutical composition is supersaturated in LD. In particular embodiments the solubility of LD in the pharmaceutical composition is at least 5 g per liter at about 25° C., at least 10 g per liter at about 25° C., or at least 15 g per liter at about 25° C. In some embodiments less than 10% of the LD prodrug acid addition salt is hydrolyzed when the pharmaceutical composition is stored at 5±3° C. for a period of 6 months. In still other embodiments the pharmaceutical composition remains substantially free of precipitated solid LD for at least 6 months when stored at about 4° C. for at least 12 months; when stored at about 4° C. for at least 18 months; when stored at about 4° C. for at least 24 months; when stored at about 4° C. for at least 3 months; when stored at about 25° C. for at least 6 months; when stored at about 25° C. for at least 12 months; when stored at about 25° C. for at least 18 months; when stored at about 25° C. for at least 24 hours, when stored at about 25° C. for at least 48 hours; when stored at about 25° C., or for at least 24 hours when stored at about 37° C. In some embodiments, the pharmaceutical composition remains substantially free of precipitated solid LD when thawed after being stored frozen for at least 3 months, or after being stored frozen for at least 12 months.

In one embodiment, the invention includes subcutaneously infusible LD prodrug acid addition salt (e.g., an acid addition salt of LDEE) pharmaceutical compositions of pH 2.1-3.9 whose production is completed shortly prior to use by the patient, caregiver, pharmacist or medical professional. Examples of such of methods of completion of production shortly prior to use include: addition of an acid or base to an LD prodrug acid addition salt solution to adjust its pH to between 2.1 and 3.9; addition of water to an LD prodrug acid addition salt solution or solid to achieve a concentration of 0.15-1.6 moles per liter LD prodrug acid addition salt; or addition of other excipients. For some embodiments, this method may be necessary in order to achieve the required shelf life.

It would be preferable to eliminate the task of completion of production of the subcutaneously infusible pharmaceutical composition shortly prior to use. In a preferred embodiment the LD prodrug acid addition salt (e.g., an acid addition salt of LDEE) pharmaceutical composition of pH 2.1-3.9 and 0.15 M to 1.6 M LD prodrug acid addition salt concentration is a ready-to-administer pharmaceutical composition, which is stored and subsequently administered (i.e., without the need to raise the pH, dilute with water, etc.). The LD prodrug acid addition salt concentration can be, for example, between 0.2 M and 0.3 M, 0.3 M and 0.4 M, 0.4 M and 0.5 M, 0.5 M and 0.6 M, 0.6 M and 0.7 M, 0.7 M and 0.8 M, 0.8 M and 1.0 M; or 1.0 M and 1.5 M. The pH can be, for example, between 2.3 and 3.3; 2.3 and 2.9; 2.4 and 2.8; 2.5 and 3.0; 3.0 and 3.5; or between 3.5 and 3.8.

In a related aspect, the invention features a container including a pharmaceutical composition of the invention. In certain embodiments the container is substantially impermeable to oxygen, the container including an atmosphere substantially free of oxygen. In some embodiments the container is a drug reservoir of an ambulatory infusion pump.

This invention features in some of its embodiments an ambulatory infusion pump system for the treatment of PD, comprising an acidic LD-prodrug solution containing reservoir and at least one cannula or needle, or two cannulas or needles, or three cannulas or needles, or four or more cannulas or needles, in fluid communication with the drug reservoir for subcutaneously infusing the solution into a subject.

The ready-to-administer LD prodrug acid addition salt pharmaceutical composition can have a shelf life of greater than 3, 6, 12, 18, or preferably 24 months; and an operational life of greater than 8 hours, 12 hours, 24 hours, 48 hours, 72 hours, 96 hours (4 days), or 7 days. The LD prodrug acid addition salt pharmaceutical composition can be administered by infusion (e.g., subcutaneously infused into the subject via one or more ambulatory infusion pumps and/or one or more cannulas or needles as described herein).

Local accumulation of the subcutaneously infused LD-prodrug can lead to adverse effects like swelling, inflammation, nodules, granulomas or panniculitis. Granulomas can form when the immune system attempts to wall off substances that it perceives as foreign but is unable to eliminate. The present invention features compositions and methods that can reduce swelling and/or formation of granulomas or nodules at the site of infusion. Excessive local accumulation, that can cause swelling or granuloma or nodule formation, can lead to an increase in the plasma LD concentration 30 min, 45 minutes or 1 hour after cessation of the infusion. It was discovered that subcutaneous depot formation, which can sustain or increase the plasma LD concentration after cessation of the infusion, can be disadvantageous because it can cause swelling or granulomas or nodules. According to the present invention, the subcutaneously infused LD-prodrug pharmaceutical compositions and methods are such that 30 min, 45 minutes or 1 hour after cessation of the subcutaneous infusion, and in absence of oral or other LD or LD-prodrug administration, the plasma concentration of LD has decreased, has not increased, or has increased by less than 50 ng/mL, 100 ng/mL, 150 ng/mL, 200 ng/mL, 250 ng/mL or 300 ng/mL from the plasma concentration at the end of the infusion.

This invention also features a method for treating Parkinson's disease in a subject by continuously or intermittently, subcutaneously infusing into the subject an L-DOPA prodrug pharmaceutical composition of pH 2.1-3.9, such as an LDEE pharmaceutical composition, at an average hourly rate of more than 100, 200, or 500 micromoles per hour. The pH of the infused acidic L-DOPA prodrug pharmaceutical composition can be between pH 2.1 and pH 3.9, e.g., pH 2.4±0.3; pH 2.6±0.3; pH 2.8±0.3; pH 3.1±0.3; pH 3.4±0.3 or pH 3.7±0.3. The acidic pharmaceutical composition can be aqueous and can comprise a salt of an L-DOPA prodrug, such as that of an ester or an amide of L-DOPA, exemplified by the hydrochloride salt of the ethyl ester of L-DOPA, LDEE.HCl. The concentration of the infused L-DOPA prodrug in the acidic pharmaceutical composition can be between about 0.1 M and about 1.5 M, for example 0.1±0.05 M; 0.2±0.1 M; 0.3±0.2 M; 0.4±0.2 M; 0.5±0.2 M; 0.6±0.3 M; 0.7±0.3 M; 0.8±0.4 M; 0.9±0.4 M; or 1.0±0.5 M.

The invention features a method for treating Parkinson's disease in a subject by subcutaneously infusing into the subject an LDEE or LDME solution in an amount sufficient to treat the Parkinson's disease, wherein the LDEE or LDME solution has a pH of 3.3±0.6 and includes from 0.25 M to 0.75 M LDEE or LDME. In particular embodiments, the LDEE or LDME solution is substantially free of precipitated solid LD when stored for 48 hours at about 25° C. In still other embodiments, the LDEE or LDME solution is substantially free of precipitated solid LD when stored for 3 months at about 5±3° C. and when subsequently stored for 16 hours at 37° C.

Embodiments of this invention comprise a method for treating PD in a subject comprising subcutaneously infusing into a subject a LD-prodrug solution of a pH between 2.1 and 3.9 in an amount sufficient to treat PD wherein the flow rate at an infused site is between 0.1 mL per hour and 2.5 mL per hour. When the pH is from 2.4 to 3.9, and the flow rate at an infused site is greater than 0.3 mL per hour, the infusion can be substantially painless. Furthermore, less than one tenth ( 1/10^{th}) of the infused sites can be swollen, inflamed or hard 24 hours or more after the infusion. In particular embodiments, the administration regimen includes a continuous infusion regimen. In still other embodiments, the administration regimen comprises an intermittent infusion regimen.

In a further aspect of the method, the average hourly infusion rate is achieved by subcutaneously infusing the acidic L-DOPA prodrug pharmaceutical composition at one site with a single cannula or needle, or at multiple skin sites with multiple cannulas or needles. For example the acidic pharmaceutical composition can be infused at two sites with two cannulas or needles, using for example a bifurcated infusion set; or at three sites with three cannulas or needles, using for example a trifurcated infusion set, or at four sites with four cannulas or needles using for example a quadrifurcated infusion set, or at more than four sites with more than four cannulas or needles. Preferably, each of the infusion sites is separated from each of the other infusion sites by a distance of greater than 1 cm (e.g., from 1 to 6 cm, from 1 to 3 cm, from 2 to 4 cm, or from 3 to 6 cm). For example, the method can include subcutaneous infusion of the pharmaceutical composition at two, three, four or greater than four infusion sites during a period of less than or equal to 24 hours (e.g., using a multifurcated infusion set, such as a bifurcated, trifurcated or quadrifurcated infusion set). The combined flow rates at the infusion sites can be greater than about 0.4 mL per hour, for example they can be greater than 0.7 mL per hour, e.g. greater than 1 mL/hour, 1.5 mL/hour, or 2 mL/hour. In particular embodiments the method include subcutaneously infusing the LD prodrug pharmaceutical composition for a period of 8 hours or more, and/or subcutaneously infusing into the subject an LD prodrug pharmaceutical composition at such a rate that: (i) a circulating plasma LD concentration greater than 400 ng/mL is continuously maintained for a period of at least 8 hours during the infusion; and (ii) at 60 minutes after the end of the infusion the plasma LD concentration is not greater than it was at the end of the infusion.

In an embodiment of any of the methods of the invention, 45 minutes after the end of the infusion the plasma LD concentration is not greater than it was at the end of the infusion, 30 minutes after the end of the infusion the plasma LD concentration is not greater than it was at the end of the infusion, the circulating plasma concentration of the LD prodrug during the infusion does not exceed 100 ng/mL, or the pharmaceutical composition is subcutaneously infused at such a rate that the circulating plasma concentration of the LD prodrug during the infusion does not exceed 50 ng/mL, 30 ng/mL, or 15 ng/mL.

In an embodiment of any of the methods of the invention, the subject receives an average daily dose of less than 20 mL of the LD prodrug pharmaceutical composition; the average daily dose is greater than 5 mL; during the infusion the circulating LD plasma concentration varies by less than +/−20% or by less than +/−10% from its mean for a period of at least 1 hour; or the average circulating plasma concentration of the LD prodrug is less than 1/500th of the average circulating plasma concentration of L-DOPA.

In any of the above methods, the method can further include administering to the subject LD, or a prodrug of LD, via a route of administration other than subcutaneous infusion. In particular embodiments, the method further includes orally administering to the subject LD or a prodrug of LD. For example, 50-100 mg, 100-200 mg, 200-300 mg, or greater than 300 mg of LD can be orally administered to the patient within one hour before or after initiating an infusion of the LD prodrug pharmaceutical composition. In particular embodiments, (i) doses of at least 50 mg or 100 mg of LD are orally administered to the patient at three or more times during the day, each dose being separated from a previous dose by at least 2 hours; and (ii) the total dose of oral LD administered during a 24 hour period is less than three times the molar dose of the infused LD prodrug pharmaceutical composition during the 24 hour period. In one particular embodiment, the method further includes administering to the subject LD, or a prodrug of LD, via pulmonary delivery. For example, 25-50 mg, 50-100 mg, 100-200 mg, or 200-300 mg of LD can be administered to the patient via pulmonary delivery within one hour before or after initiating an infusion of the LD prodrug pharmaceutical composition. In particular embodiments, (i) doses of at least 50 mg or 100 mg of LD are administered to the patient via pulmonary delivery at three or more times during the day, each dose being separated from a previous dose by at least 2 hours; and (ii) the total dose of LD administered via pulmonary delivery during a 24 hour period is less than three times the molar dose of the infused LD prodrug pharmaceutical composition during the 24 hour period. In particular embodiments the average daily molar amount of infused LD prodrug acid addition salt is less than 1.6 times, less than 1.2 times, less than 1.0 times, or less than 0.8 times the average daily molar amount of oral LD taken by the patient when not infusing the LD prodrug acid addition salt.

In one particular embodiment the LD prodrug acid addition salt is subcutaneously infused into the subject at one or more infusion sites, wherein the infusion volume at each of the infusion sites is less than 20 mL over a 24 hour period and the amount of LD prodrug acid addition salt administered at each of the infusion sites is less than 10, 5, or 3 millimoles over a 24 hour period.

The subcutaneously infusible acidic pharmaceutical composition of pH 2.1-3.9 can be stored without L-DOPA precipitation when refrigerated at 5±3° C., for example at about 4° C., for at least 3 months, 6 months, 12 months, 18 months, or 24 months. It can also be stored at about 37° C. for at least 8 hours, for example for at least 16 or 24 hours without L-DOPA precipitation.

Typically the shelf life of the subcutaneously infusible acidic pharmaceutical compositions is at least 3 months, for example at least 6 months, at least 12 months, or at least 24 months when the pharmaceutical compositions are stored at about 4° C. Their operational life at about 37° C. is at least 8 hrs, for example at least 16, 24 hours or 48 hours.

In particular embodiments, the acidic LDEE pharmaceutical composition remains substantially free of precipitated solid LD when thawed after being stored frozen (e.g., at about −18° C. or at about −3° C.) for at least 3 months, 6 months, 12 months, 18 months, or 24 months.

The solubility of LD formed upon hydrolysis of the prodrug increases when the pharmaceutical composition is made increasingly acidic, i.e., when the pH is lowered. Such increased solubility is important as it increases the shelf life and operational life of the pharmaceutical composition. In some embodiments, the ready-to-infuse pharmaceutical composition, can be sufficiently acidic to remain free of precipitated LD, e.g., not light scattering, or opaque when stored at about 25° C. for at least 3 months, e.g., for at least 6 months, for at least 12 months or for at least 18 months.

In some embodiments, filtration of the pharmaceutical composition, e.g., for its sterilization, can remove nucleating particles such that the infused acidic pharmaceutical composition can be supersaturated in LD and no solid LD will precipitate when the thermodynamic solubility limit is reached. Such an LD supersaturated solution can also remain free of precipitated solid, e.g., not light scattering or opaque, when stored at about 25° C. for at least 3 months, for example at least 6 months, 12 months or 18 months.

In particular embodiments, the sum of the LD prodrug acid addition salt administered over all sites over a 24 hour period is less than about 20 millimoles (e.g., 0.2 to 1, 0.5 to 5, or 3 to 7 or, 5 to 8, or 7 to 12, or 10 to 14, or 12 to 16, 15 to 20 millimoles) and the total infusion volume, i.e., the sum of the infusion volume administered over all sites over a 24 hour period is less than 40 mL, 35 mL, 30 mL, 25 mL, 20 mL, 15 mL, or 10 mL. For example, the sum of the LD prodrug acid addition salt administered over all sites over a 24 hour period can be from 1 to 20 millimoles (e.g., 1 to 3 millimoles, 3 to 6 millimoles, 6 to 10 millimoles, 10 to 15 millimoles, or 15 to 20 millimoles) and the sum of infusion volume administered over all sites over a 24 hour period can be between 3 and 40 mL (e.g., between 3 and 6 mL, 5 and 16 mL, 10 and 16 mL, 16 and 24 mL, 20 and 30 mL, or 30 and 40 mL) over the 24 hour period.

In one embodiment, a LD prodrug acid addition salt, such as an acid addition salt of LDEE or LDME, is infused continuously or intermittently (at least once every 60-120 minutes) over a period of at least 8 hours. The LD prodrug acid addition salt can be infused in an amount sufficient to maintain a circulating plasma LD concentration greater than 400 ng/mL (e.g., greater than 400, 800, 1200, or 1600) and less than 7,500 ng/mL (e.g., less than 5,000 ng/mL, 2,500 ng/mL, or 2,000 ng/mL), which is continuously maintained in the subject for a period of at least 8 hours. The LD prodrug acid addition salt can also be infused in an amount sufficient to maintain a circulating plasma LD concentration greater than 400 ng/mL (e.g., greater than 400, 800, 1200, or 1600) and less than 7,500 ng/mL (e.g., less than 5,000 ng/mL, 2,500 ng/mL, or 2,000 ng/mL), which is continuously maintained in the subject for a period of at least 8 hours. In a preferred version of the method of infusion, the acidic LD prodrug pharmaceutical composition is subcutaneously infused at such a rate that the circulating LD plasma concentration varies by less than +/−20%, +/−15%, or +/−10% from its mean for a period of at least 1 hour, 2 hours, 4 hours, or 8 hours. At the end of the infusion the plasma concentration typically decays. The circulating plasma LD concentration can decay already 30 min, 45 min, or 60 minutes after the end of the infusion and must not increase by more than 50 ng/mL, 100 ng/mL, 150 ng/mL, 200 ng/mL, 250 ng/mL or 300 ng/mL at these times.

In a preferred embodiment, the LD-prodrug, which can be a water-soluble salt (e.g., LDEE.HCl), is rapidly converted to L-DOPA, such that during most of the infusion period the plasma concentration of the infused prodrug is at least 100 times (one hundred times) less than the plasma concentration of L-DOPA.

Optionally, the subject may also take LD or and LD prodrug acid addition salt orally in conjunction with the infusion. For example, a subject who did not infuse the LD prodrug acid addition salt at night can be administered an oral LD dose when waking up in the morning, optionally at about the time the subcutaneous infusion of the LD prodrug acid addition salt is started.

The invention further features a container including a reconstitutable solid which can be mixed with an aqueous acid, such as HCl, to form a ready-to-administer LD prodrug acid addition salt pharmaceutical composition having a pH not greater than about 3.9 and not less than about 2.0 (e.g., between 3.5 and 3.8 or between 3.0 and 3.5, or between 2.5 and 3.0, or between 2.4 and 2.8, or between 2.3 and 3.3, or between 2.3 and 2.9). In certain embodiments, the reconstitutable solid is substantially free of water. In other embodiments, the reconstitutable solid includes a buffering agent (e.g., citric acid, succinic acid, citrate, or any other suitable buffer described herein). In some embodiments, the reconstitutable solid includes free base LDEE and/or the LDEE salt.

The invention also features a method of preparing an infusible pharmaceutical composition by dissolving in 5 minutes or less, at about 25° C., solid free base LDE or LDA and/or a salt thereof and a solid polybasic acid and/or salt of a polybasic acid (of an at least tenfold lesser molar amount than the molar amount of the LDE or the LDA) stored in a first container; by adding to the solid mixture HCl of a concentration of less than 2 M, 1.5 M, 1M, 0.75M, 0.6 M or 0.5 M stored in a second container, such that the pH of the resulting pharmaceutical composition is between 2.1 and 3.9. This pharmaceutical composition remains clear, i.e., precipitate-free, when kept at about 25° C. for more than 48 hours or longer or at 37° C. for more than 16 hours. Exemplary free bases and/or salts are those of LDEs, including LDEE and LDME. Exemplary polybasic acids include citric acid and succinic acid; exemplary polybasic acid salts include trisodium citrate, disodium citrate, trisodium phosphate or disodium phosphate.

In a related aspect, the invention features a method for treating Parkinson's disease in a subject by (i) reconstituting a reconstitutable solid or liquid with water to form an LDEE pharmaceutical composition having a pH not greater than about 3.9 and not less than about 2.0 (e.g., between 3.5 and 3.8 or between 3.0 and 3.5, or between 2.5 and 3.0, or between 2.4 and 2.8, or between 2.3 and 3.3, or between 2.3 and 2.9, and including from 0.15 M to 1.5 M LDEE (e.g., 0.15±0.05 M, 0.25±0.05 M, 0.35±0.05 M, 0.45±0.05 M, 0.55±0.05 M, 0.65±0.05 M, 0.75±0.05 M, 0.85±0.05 M, or 1.25±0.25 M LDEE); and (ii) subcutaneously infusing the LDEE pharmaceutical composition into the subject in an amount sufficient to treat the Parkinson's disease. In certain embodiments, the reconstitutable solid or liquid is substantially free of water. In other embodiments, the reconstitutable solid includes a buffer (e.g., citric acid, citrate, or any other suitable buffer described herein). The method can include subcutaneous infusion at one or more sites on a subject. In particular embodiments, the sum of the LDEE administered over all sites over a 24 hour period is less than about 20 millimoles (e.g., 0.2 to 1, 0.5 to 5, or 3 to 7 or, 5 to 8, or 7 to 12, or 10 to 14, or 12 to 16, 15 to 20 millimoles) and the total infusion volume, i.e., the sum of the infusion volume administered over all sites over a 24 hour period is less than 40 mL, 35 mL, 30 mL, 25 mL, 20 mL, 15 mL, or 10 mL. For example, the sum of the LDEE administered over all sites over a 24 hour period can be from 1 to 20 millimoles (e.g., 1 to 3 millimoles, 3 to 6 millimoles, 6 to 10 millimoles, 10 to 15 millimoles, or 15 to 20 millimoles) and the sum of infusion volume administered over all sites over a 24 hour period can be between 3 and 40 mL (e.g., between 3 and 6 mL, 5 and 16 mL, 10 and 16 mL, 16 and 24 mL, 20 and 30 mL, or 30 and 40 mL) over the 24 hour period.

The subcutaneously infusible pharmaceutical composition of pH 2.1-3.9 can further include a pharmaceutically acceptable excipient, such as a crystal growth inhibitor and/or antioxidants. In particular embodiments, the LD prodrug acid addition salt is a hydrochloride salt. In still other embodiments, the infused liquid composition has a viscosity of between 1.2 cP and 2,000 cP (e.g., from 1.2 cP to 2 cP, 1.5 cP to 5 cP, 2.5 cP to 7.5 cP, 5 cP to 10 cP, 1.2 cP to 200 cP, 10 cP to 200 cP, or 200 cP to 2,000 cP). The pharmaceutical composition can be substantially free of oxygen. In particular embodiments, the liquid includes a polycarboxylate (e.g., hyaluronic acid, succinylated gelatin, poly(acrylic acid), poly(methacrylic acid), poly(glutamic acid), poly(aspartic acid), poly(maleic acid), poly(malic acid), or poly(fumaric acid)). In still other embodiments, the LD prodrug acid addition salt is an acid addition salt of hydrochloric acid, sulfuric acid, or phosphoric acid. In certain embodiments the pharmaceutical composition is a liquid that is supersaturated in LD. In particular embodiments, the solubility of LD in the pharmaceutical composition is at least 5 g per liter at about 25° C.

In particular embodiments, the container can include a pharmaceutically acceptable excipient, such as a viscosity enhancing agent, an anti oxidant, and/or a preservative. For example, the container can further include from 0.5 to 4.0% (w/w) hyaluronic acid, or any other viscosity enhancing agent described herein; and/or the container can further include an antioxidant (e.g., bisulfite, propofol, salicylic acid or salicylic acid salt, a salt of ascorbic acid (such as sodium ascorbate), p-aminophenol, acetamol, a t-butyl ortho-substituted phenol, or any antioxidant described herein). In any of the above pharmaceutical compositions, the LD prodrug acid addition salt can be selected from acid addition salts of LDAs and LDEs. In particular embodiments, the LD prodrug acid addition salt is an acid addition salt of LDEE or LDME, such as LDEE hydrochloride salt.

The invention further features a container including a material that is substantially impermeable to oxygen, the container containing a reconstitutable solid including an LD prodrug salt, or its free base, wherein the container is substantially free of oxygen and wherein the reconstitutable solid, when reconstituted, is suitable for subcutaneous infusion. The invention also features a container including a material that is substantially impermeable to oxygen, the container containing liquid including an LD prodrug, wherein the container is substantially free of oxygen and wherein the liquid is suitable for subcutaneous infusion. In general, the amount of oxygen permeated annually through the walls of the container containing the daily dose of a patient and residing in ambient air at about 5±3° C., for example at 4±1° C., is less than 0.5 millimoles, for example less than 0.4, 0.3, 0.2, 0.1, 0.05, 0.025 millimoles, i.e., generally less than about 10 mL of the gas.

In particular embodiments, the LD prodrug in the container is an LDE such as an LDEE (e.g., LDEE.HCl). In certain embodiments, the container is designed to hold less than 50 mL, 40 mL, 35 mL, 30 mL, 25 mL, 20 mL, 15 mL, 10 mL, 5 mL, 3 mL of a liquid including from 0.15 M to 1.6 M LD prodrug (e.g., 0.25±0.1; 0.4±0.1, 0.5±0.1, 0.6±0.1, 0.7±0.1, 0.8±0.2, 1.0±0.3, 0.8±0.3, 1.0±0.5, or 1.5±0.5 moles per liter) and having a pH of from 2.1 to 3.9, wherein the container is substantially free of oxygen.

In a highly preferred embodiment, the container in which a pharmaceutical composition of the invention is stored also functions as the drug reservoir of an ambulatory infusion pump. Such an arrangement eliminates the need for the patient or caregiver to transfer the drug from the storage container to the drug reservoir.

In a related aspect, the invention features one or more ambulatory infusion pumps for the treatment of Parkinson's disease in a subject, each comprising a drug reservoir containing an LD prodrug pharmaceutical composition (e.g., an LDE or LDEE pharmaceutical composition) of the invention. In a further embodiment, one or more implantable cannulas or needles are placed in fluid communication with the drug reservoir(s) for subcutaneously infusing the LD prodrug pharmaceutical composition into the subject.

The pharmaceutical composition can be infused into the subject using a bifurcated, trifurcated, quadrifurcated (tetrafurcated) or multifurcated infusion set bearing a plurality of cannulas positioned at a plurality of sites on the subject. Their cannulas or needles can be separated from each other by at least about 1 cm, for example by more than 2 cm, 3 cm, 4 cm, or 5 cm.

Optionally the system can include a software unit including a program for controlled infusion of the LD prodrug pharmaceutical composition.

The devices of the invention can further include a timer, an actuator, software, memory, a data processing unit, and information input/output capability, wherein the system is able to input, store and recall data including one or more of the subject's symptoms or drug responses related to Parkinson's disease, such symptoms selected from the group of tremor, hyperkinesia, dystonia, akinesia, bradykinesia, tremor, turning on, turning off, delayed time to on, and response failure. In a particular embodiment, the ambulatory infusion pump system can further include software, memory, a data processing unit, and user input capability to input into the system information related to the ingestion of a meal, and the system thereafter adjusts the rate of infusion of the pharmaceutical composition. For example, the pump system can be programmed to increase the rate of infusion after a meal including protein. In still other embodiments, the ambulatory infusion pump system can further include a timer, an actuator, software, memory, a data processing unit, and information input/output capability, wherein the system is able to automatically increase the rate of infusion of the pharmaceutical composition, by a factor of two or more, at a preset time in the morning or after a period of at least four hours. In still another embodiment, the ambulatory infusion pump system can further include a data processing unit; and a motion sensor electrically connected to, or in RF communication with, the data processing unit to detect movement of the subject, wherein the system recommends a change in the infusion rate in response to the data from the motion sensor.

In a highly preferred embodiment, the container in which a pharmaceutical composition of the invention is stored also functions as the drug reservoir of an ambulatory infusion pump. Such an arrangement eliminates the need for the patient or caregiver to transfer the drug from the storage container to the drug reservoir.

The invention further features a pharmaceutical composition including greater than 0.15 M LD prodrug acid addition salt (e.g., 0.25±0.1; 0.3±0.1, 0.4±0.1, 0.5±0.1, 0.6±0.1, 0.7±0.1, 0.8±0.2, 1.0±0.3, 1.0±0.5 or 1.5±0.5 prodrug); greater than 0.05 M benserazide or carbidopa prodrug salt (e.g., 0.06±0.1, 0.15±0.1, 0.25±0.2, 0.5±0.3 carbidopa prodrug salt), and water.

In a highly preferred embodiment, the subcutaneously infused acidic pharmaceutical composition includes LDEE at a free-base concentration between 50 and 200 mg/mL (e.g., 100-150 mg/mL) and a pH between 2.0 and 4.0, e.g., between 2.4 and 3.7. This pharmaceutical composition is infused typically subcutaneously at a depth of 1 to 17 mm (e.g., 5-10 mm) below the surface of the skin.

The invention features a pharmaceutical composition including an aqueous solution containing (i) from 0.15 to 1.6 M LD prodrug acid addition salt, (ii) greater than 0.05 M carbidopa prodrug salt or benserazide salt, and (iii) having a pH of from 2.1 to 3.9, wherein the pharmaceutical composition is subcutaneously infusible.

The invention further features a pharmaceutical composition including an aqueous solution containing from 0.15 to 1.6 M LD prodrug acid addition salt, and having a pH of from 2.1 to 3.9, wherein the pharmaceutical composition is subcutaneously infusible, and wherein the pharmaceutical composition remains substantially free of LD precipitate for at least 24 hours when stored at about 37° C.

The invention features an LDEE or LDME solution having a pH of 3.3±0.6 and including from 0.25 M to 0.75 M LDEE, LDME, or a salt thereof.

The invention further features an LDEE solution having a pH of 3.7±0.3 or a pH of 3.5±0.3 (e.g., 3.5±0.2 or 3.5±0.1) or a pH of 3.2±0.3 (e.g., 3.2±0.2 or 3.2±0.1) or a pH 2.9±0.3 (e.g., 2.9±0.2 or 2.9±0.1) and including from 0.15 M to 1.5M LDEE, or a salt thereof.

The invention also features a kit including: (i) a first container including a sterile aqueous solution; (ii) a second container including a sterile, dry, reconstitutable solid; and (iii) instructions for combining the contents of the first container with the contents of the second container to form a pharmaceutical composition suitable for subcutaneous infusion into a subject and for infusing the pharmaceutical composition into a subject for the treatment of Parkinson's disease; wherein the solid fully dissolves in the solution in less than 5 minutes at 25° C.; the infusible pharmaceutical composition includes LDEE and has a pH of from 2.1 to 3.9; and less than 10% of the LDEE is hydrolyzed when the first container and the second container are stored at 5±3° C. for a period of 3 months. In particular embodiments, subsequent to storage of the first container and the second container at 5±3° C. for a period of 3 months and then forming the infusible pharmaceutical composition, the infusible pharmaceutical composition remains substantially free of precipitated LD when kept at about 37° C. for at least 24 hours. In certain embodiments, the sterile, dry, reconstitutable solid includes LDEE.

The invention features a method for treating Parkinson's disease in a subject, the method including subcutaneously infusing into the subject a pharmaceutical composition including LDEE in an amount sufficient to treat the Parkinson's disease, wherein the pharmaceutical composition has a pH of 3.1±0.8 and includes from 0.15 M to 1.6 M LDEE.

The invention features a method for subcutaneously infusing a pharmaceutical composition including the steps of: (i) providing a subcutaneously infusible, aqueous pharmaceutical composition containing 0.15 M-1.6 M LD prodrug acid addition salt and a pH of from 2.1 to 3.9, wherein less than 10% of the LD prodrug acid addition salt is hydrolyzed when the pharmaceutical composition is stored at 5±3° C. for a period of 6 months; and (ii) inserting the infusible pharmaceutical composition into an infusion pump, wherein the pharmaceutical composition remains substantially free of precipitated LD when kept at about 25° C. for at least 24 hours. The infusible pharmaceutical composition can include a pharmaceutical composition of the invention.

In any of the above methods, the method can further include subcutaneously infusing into the subject the pharmaceutical composition in a pulsed dosing regimen, wherein the pulsed dosing regimen includes (i) a delivery period during which the LD prodrug solution is infused at a first site for from 1 second to 3 hours (e.g., 1-10, 10-100, 100-200, 200-400, or 400-800 seconds, 10 minutes to 30 minutes, 30 minutes to 1 hour, 1 hour to 2 hours, or 2 hours to 3 hours); and (ii) following step (i), a non-delivery period during which the LD prodrug solution is administered at a substantially reduced rate at the first site for from 10 to 120 minutes, and repeating steps (i) and (ii). In particular embodiments the non-delivery period is from 10 to 60 minutes. In still other embodiments, the non-delivery period is from 60 minutes to 120 minutes. The ratio of the length of the delivery period to the length of the non-delivery period can be from 1:4 to 4:1. In certain embodiments, the flow rate during the delivery period is 0.35±0.05 mL/hour. The LD prodrug solution can include 0.25 to 1.6 M LDEE.HCl, optionally buffered with citrate.

The invention features a method for treating Parkinson's disease in a subject, the method including: (i) subcutaneously infusing into the subject a LD prodrug acid addition salt; and (ii) delivering LD, or a prodrug of LD, via a second route of administration other than subcutaneous infusion, wherein (a) 50-500 mg (e.g., 50-100, 100-200, 200-300, or 300-500 mg) of LD, or a prodrug of LD, is administered to the patient via the second route of administration within one hour before or after initiating an infusion of the LD prodrug pharmaceutical composition; and (b) a circulating plasma LD concentration less than 5,000 ng/mL is continuously maintained for a period of at least 8 hours during the infusion. In particular embodiments, the second route of administration is oral administration. In still other embodiments, the second route of administration is pulmonary or transcutaneous administration.

The invention further features a method for treating Parkinson's disease in a subject, the method including: (i) subcutaneously infusing into the subject a LD prodrug acid addition salt; and (ii) delivering LD, or a prodrug of LD, via a second route of administration other than subcutaneous infusion, wherein (a) doses of 50-500 mg (e.g., 50-100, 100-400, 200-300, or 300-500 mg) of LD, or a prodrug of LD, are administered to the patient via the second route of administration at three or more times during the day, each dose being separated from a previous dose by at least 2 hours; and (b) the total dose of LD, or a prodrug of LD, administered to the patient via the second route of administration during a 24 hour period is less than three times (e.g., less than two times, less than one times, less than 50%, or less than 25%) the molar dose of the infused LD prodrug acid addition salt during the 24 hour period. In particular embodiments, the second route of administration is oral administration. In still other embodiments, the second route of administration is pulmonary or transcutaneous administration.

The invention further features a kit including (i) a pharmaceutical composition of the invention; and (ii) instructions for administering the composition to a subject for the treatment of Parkinson's disease.

The invention features a method for using the pharmaceutical composition of the invention, the method including the step of visually inspecting the composition prior to use to determine whether the pharmaceutical composition is suitable for infusion into a subject, wherein a transparent pharmaceutical composition is suitable for infusion and a colored, or light scattering, or opaque pharmaceutical composition is not suitable for infusion. In certain embodiments the pharmaceutical composition is packed in a kit or container that is configured to permit visual inspection of the pharmaceutical composition.

The invention features a method of manufacturing the pharmaceutical composition of the invention, including dissolving dry crystallites of an LD prodrug or its free base in an aqueous solution.

In some of its further embodiments this invention features subcutaneously infused aqueous pharmaceutical compositions comprising a therapeutic agent and having a pH of from 2.4 to 3.0 that is infused at a rate greater than 0.01 mL per hour per infused site, e.g. greater than 0.1 mL per hour per infused site, or greater than 0.3 mL per hour per infused site, wherein fewer than 1/10^{th} of the infused sites become inflamed, swollen or hard 24 hours or more after the infusion. The infusion can be substantially painless. The therapeutic agent can treat a disease and/or alleviate its symptoms; it can, for example, treat or alleviate symptoms of PD.

In any of the above methods, compositions, and kits the LD prodrug acid addition salt can be an acid addition salt of levodopa ethyl ester or levodopa methyl ester.

In any of the above methods for treating Parkinson's disease, the method can be used to alleviate a motor or non-motor complication in a subject afflicted with Parkinson's disease, such as tremor, akinesia, bradykinesia, dyskinesia, dystonia, cognitive impairment, and disordered sleep. The method can further include administration of an effective amount of an anti-emetic agent (e.g., nicotine, lobeline sulfate, pipamazine, oxypendyl hydrochloride, ondansetron, buclizine hydrochloride, cyclizine hydrochloride, dimenhydrinate, scopolamine, metopimazine, or diphenidol hydrochloride). For example, the methods can include administering an effective amount of benserazide or carbidopa prodrug (e.g., orally or by infusion). Examples of carbidopa prodrugs include its esters, such as carbidopa ethyl ester and carbidopa methyl ester, and carbidopa amide, the highly water soluble hydrochloride salts of which are preferred as carbidopa prodrugs. In either of the above methods, the pharmaceutical composition can administered by subcutaneous infusion or intramuscular infusion. For example, the pharmaceutical composition can be infused proximate a large muscle (e.g., the diaphragm, trapezius, deltoid, pectoralis major, triceps brachii, biceps, gluteus maximus, sartorius, biceps femoris, rectus femoris, and gastrocnemius) at a depth between 3 mm and 15 mm below the stratum corneum of the subject (e.g., 3 mm to 5 mm, 5 mm to 7 mm, or 7 mm to 9 mm beneath the stratum corneum of the subject). In particular embodiments the pharmaceutical composition is co-infused with an extracellular matrix degrading enzyme (e.g., a hyaluronidase) and/or with an analgesic (e.g., salicylic acid, or a salt thereof), or an analgesic is topically administered to the subject at the site of administration.

The devices of the invention can further include can further include a timer, an actuator, software, memory, a data processing unit, and information input/output capability, wherein the system is able to input, store and recall data including one or more of the subject's symptoms or drug responses related to Parkinson's disease, such symptoms selected from the group of tremor, hyperkinesia, dystonia, akinesia, bradykinesia, tremor, turning on, turning off, delayed time to on, and response failure. In a particular embodiment, the ambulatory infusion pump system can further include software, memory, a data processing unit, and user input capability to input into the system information related to the ingestion of a meal, and the system thereafter adjusts the rate of infusion of the pharmaceutical composition. For example, the pump system can be programmed to increase the rate of infusion after a meal including protein. In still other embodiments, the ambulatory infusion pump system can further include a timer, an actuator, software, memory, a data processing unit, and information input/output capability, wherein the system is able to automatically increase the rate of infusion of the pharmaceutical composition, by a factor of two or more, at a preset time in the morning or after a period of at least four hours. In still another embodiment, the ambulatory infusion pump system can further include a data processing unit; and a motion sensor electrically connected to, or in RF communication with, the data processing unit to detect movement of the subject, wherein the system recommends a change in the infusion rate in response to the data from the motion sensor.

The devices of the invention can further include a pharmaceutical composition including an aqueous liquid containing an LD prodrug, or a salt thereof, and water, wherein the weight percent of water in the pharmaceutical composition is less than the weight percent of said LD prodrug, or a salt thereof (e.g., by mass the ratio of LD prodrug, or a salt thereof, to water is from 1.05:1.0 to 1.25:1.0; 1.15:1.0 to 1.55:1.0; 1.25:1.0 to 1.75:1.0; 1.75:1.0 to 2.0:1.0; 1.85:1.0 to 3.0:1.0; or from 2.0:1.0 to 4.0:1.0).

The devices of the invention can further include a pharmaceutical composition including an aqueous liquid containing an LD prodrug, or a salt thereof, and water, wherein the aqueous liquid has a density between 1.15 and 1.95 g/mL (e.g., a density of from 1.15 to 1.45, 1.25 to 1.65, or 1.35 to 1.95 g/mL) at about 25° C.

The invention further features a pharmaceutical composition including greater than 0.15 M LD prodrug, or a salt thereof (e.g., 0.25±0.1; 0.3±0.1, 0.4±0.1, 0.5±0.1, 0.6±0.1, 0.7±0.1, 0.8±0.2, 1.0±0.3, 1.0±0.5, 1.5±0.5, 2.0±0.5, 2.5±0.5, 3.0±0.5, or 3.5±0.5 M LD prodrug); greater than 0.05 M carbidopa prodrug (e.g., 0.06±0.1, 0.7±0.1, 0.8±0.1, 0.9±0.1, 1.0±0.2, or 1.5±0.5 M carbidopa prodrug), or a salt thereof, and water. The pharmaceutical composition can optionally further include a vasoactive compound, such as a venous vasodilator.

In any of the above methods, the LD prodrug, or a salt thereof, can be infused intragastrically, intraduodenally or intrajejunally through a tube of less than about 3 mm, 2 mm, 1.5 mm, 1.0 mm outer diameter, and/or an internal diameter of less than 1 mm, 0.7 mm, 0.35 mm for a period of greater than or equal to about 12 hours, 24 hours, 48 hours, 72 hours, and most preferably 96 hours.

In any of the above methods, the LD prodrug, or a salt thereof, can be co-infused with an agent, or a prodrug thereof, in an amount sufficient to reduce local swelling, inflammation, or granuloma formation.

In a preferred embodiment, the infused solution includes a LDEE concentration between 50 and 200 mg/mL (e.g., 100-150 mg/mL) and a pH between 3.0 and 4.5 (e.g., 3.0-4.0). This solution is infused subcutaneously at a depth of 5 to 13 mm (e.g., 6-10 mm) below the surface of the skin.

In any of the above methods, compositions, and kits, the invention features an LD prodrug solution for storage in the range of pH 2.1-3.9 and LD prodrug solutions for infusion in the range of pH 2.5-4.6. In some instances, an LD prodrug solution may be suitable for both storage and infusion without adjusting the pH of the solution if the solution, for example, has a pH of between 2.5 and 3.9.

Other features and advantages of the invention will be apparent from the following Detailed Description and the claims.

### DETAILED DESCRIPTION

The invention features methods of subcutaneous infusion and of infused compositions for the treatment of Parkinson's disease. The methods and compositions can reduce the severity and/or rate of occurrence of subcutaneous infusion site reactions, such as persistent subcutaneous granulomas, transient swelling and erythema. The invention also features devices and methods for maintaining plasma LD concentrations in a desired therapeutic target range and can enable PD patients to reduce the variability of their plasma and/or brain LD concentrations, e.g., reducing the magnitude and/or frequency of high or low LD excursions. By controlling the LD concentrations in the body, the durations of periods and/or severities of symptoms of PD, such as off periods and periods with severe dyskinesias, are reduced. The fluctuations, leading to the devastating symptoms of the disease, are alleviated by continuous, frequent and/or programmed, subcutaneous infusion of a pharmaceutical composition including an LD prodrug. The continuous or frequent infusion stabilizes the patient's plasma LD concentration.

In the methods of the invention a therapeutic plasma L-DOPA concentration can be provided by subcutaneous infusion of an L-DOPA prodrug dissolved in an a aqueous solution, without excessive elevation of the LD-prodrug level in the circulating blood or its plasma. Upon its subcutaneous infusion, the LD-prodrug can be so rapidly hydrolyzed that the LD-prodrug concentration can be less than 1/50^{th}, for example less than 1/100^{th} or less than 1/500^{th} of the plasma LD concentration.

While hydrolysis of the LD-prodrug after its subcutaneous infusion can be rapid, the ready-to-infuse LD-prodrug is not rapidly hydrolyzed in its aqueous solution while in its storage container or drug reservoir, prior to infusion. Because the solubility of LD in water can be only 1/100^{th} or less of the solubility of the LD-prodrug, LD can precipitate if the prodrug hydrolyzes rapidly prior to its infusion. The rate of hydrolysis of the ready-to-infuse solution can be slow enough for the solution to remain precipitate-free for at least 2 days at room temperature near 25° C. or for at least one day at body temperature near 37° C.

For ease of use by a movement impaired PD patient it is desired that the infused solution and the stored, i.e. purchased or distributed, solution be the same, avoiding the need to dilute a concentrate or dissolve a solid powder. In one of its aspects the disclosed invention provides infusible solutions that can stored refrigerated at about 5±3° C., for example 4±2° C., with shelf life stability for at least 3 months, for example for 6 months, for 12 months or for 18 months. The disclosed concentrations of the LD-prodrug in the infused acidic solutions can be between 0.15 M and 1.5 M, for example between 0.2 M and 1 M, or between 0.25 M and 0.75 M.

The daily doses and the delivery rates required for managing symptoms of PD increase progressively with the disease and patients requiring daily more than 2.5 millimoles of the prodrug, for example 5 millimoles, or 10 millimoles or even 15 millimoles can be in particular need of stabilizing their plasma LD-concentrations through continuous or frequent subcutaneous infusion. Because the doses and dose rates can be high the infused tissue can be adversely affected. The methods and compositions disclosed can reduce the severity and/or rate of occurrence of infusion site reactions, such as persistent subcutaneous granulomas, transient swelling and erythema. We have discovered that the severity or frequency of the skin symptoms can be reduced when the infused solution is much more acidic than the infused tissue. The pH of the infused solution is generally between 2.1 and 4.6; it is preferably between pH 2.6 and 4.2 for example 2.6±0.3, 2.8±0.3, 3.0±0.3, 3.2±0.3, 3.4±0.3 or 3.6±0.3; 3.9±0.3.

We have also discovered that periodic interruption of the infusion can reduce the severity or frequency of the skin symptoms.

The severity or frequency of these skin symptoms can also be reduced by splitting the flow of the LD-prodrug solution, e.g., by infusing it through two or more cannulas separated by 1 cm or more, for example by using a multi-furcated infusion set connected to a pump. The flow can be divided, for example, between two infused sites by using a bifurcated infusion set, three infusion sites using a trifurcated set or four sites using a quadrifurcated set.

Infusion Site Tolerability

To manage the symptoms of Parkinson's disease in advanced patients who suffer most from the disease, their plasma L-DOPA concentration is raised typically to the range between about 400 ng/mL and about 5,000 ng/mL, often to the range between about 800 ng/mL and 2,500 ng/mL, and because the typical plasma half-life of L-DOPA in patients receiving also an inhibitor of L-DOPA decarboxylase is about 10² minutes. The combined daily molar amount of administered (e.g., subcutaneously infused, orally administered and/or inhaled) LD, or LD prodrug, can be between about 1 millimoles and about 15 millimoles; according to this invention. Of this combined daily molar amount at least 25%, i.e., between about 0.25 millimoles and about 3.75 millimoles, is subcutaneously infused; preferably more than 50%, i.e., more than between about 0.5 millimoles and 7.5 millimoles is subcutaneously infused; and more than 75% can be subcutaneously infused.

The subcutaneously infused mass of the LD-prodrug for the treatment of Parkinson's disease is at least tenfold greater than that of the mass of subcutaneously infused insulin for the treatment of diabetes. In the methods of the invention, one gram or larger quantities of an L-DOPA prodrug are infused into patients to manage the symptoms of Parkinson's disease. Subcutaneous infusion of drugs including LDEE.HCl in such high mass, and at associated high dose-rates, can lead to adverse local effects, such as tenderness, swelling, inflammation or erythema, panniculitis, or formation of nodules, indurations, and/or granulomas, e.g., if the infused drug accumulates at or near the infused site.

Improving Infusion Site Tolerability

The present invention features aqueous compositions and methods for which infusion associated pain, cellular damage and inflammation can be reduced or avoided, i.e., disclosed methods and compositions can reduce the severity and/or rate of occurrence of the subcutaneous infusion site reactions. The invention also discloses devices and methods for maintaining plasma LD concentrations in a desired therapeutic target range. Furthermore, the invention features acidic infusible pharmaceutical compositions of LD prodrugs for managing symptoms of Parkinson's disease that are stable enough to be stored refrigerated and are also stable enough to be infused at body temperature. The disclosed methods of the invention can alleviate adverse reactions of the skin and nearby tissues. These reactions can depend on the LD-prodrug dose infused at a site and/or on the dose rate for the infused site, being more severe when the dose and/or dose rate is higher. Because more advanced PD patients receive the larger doses requiring greater dose rates, the compositions and methods disclosed here could affect patients in great need of continuous subcutaneous infusion therapy.

The adverse skin effects can be alleviated by infusing pharmaceutical compositions of low pH; by pulsing, i.e., periodically interrupted infusion of the prodrug containing solution; and by maintaining the concentration of the LD-prodrug in a defined range.

Stable LD Prodrugs that are Rapidly Hydrolyzed Upon Infusion

The preferred subcutaneously administered prodrugs include highly water soluble LDEs or LDAs and their salts, which can be rapidly hydrolyzed in the body, typically in an enzyme catalyzed reaction, to form LD. Although they are rapidly hydrolyzed in the body, the operational, ready to infuse, aqueous prodrug pharmaceutical compositions can be stored in a container of the infusion system at least for 48 hours at room temperature near 25° C., or at body temperature near 37° C. for at least 16 hours, or at a temperature in between, such as about 30° C., for at least 24 hours.

The LD prodrugs are hydrolyzed to LD, which can be much less soluble in water or in aqueous solutions in the pH range suitable for subcutaneous infusion. The shelf life of the stored and of the ready-to-infuse LD prodrug pharmaceutical compositions is usually determined by their hydrolysis, leading to LD precipitation, which can be faster than the other degradation processes, such as oxidation, particularly when oxygen is substantially excluded. For this reason, a major problem with the LD prodrug formulations, particularly of the aqueous formulations, is their hydrolytic instability. The rate of hydrolysis is pH and temperature dependent. Because the LD is poorly soluble, and because the concentration of the LD prodrug in the small-volume subcutaneously infused pharmaceutical composition can be high, even hydrolysis of a small fraction of a dissolved LD prodrug may result in the precipitation of LD from the pharmaceutical composition. The presence of a large amount of LD precipitate is unacceptable, as it may lead to a dosing error and because it may block or reduce the flow in the infusion system.

At a particular temperature and pH, the LDEs formed of LD and of different alcohols are hydrolyzed prior to their infusion at different rates. For example, the rate of hydrolysis of LDEE near pH 7 and at about 37° C. can be about four times faster than the rate of hydrolysis of LDME. The rate of hydrolysis of LDE salts also depends on the anion, i.e., on the acid forming the LDE salt. LDE is hydrolyzed more rapidly when carboxylate anions and/or carboxylic acids replace the chloride anion or HCl. The rate of hydrolysis of the salt formed of LDEE and acetic acid can be about 3 times faster at about pH 4.5 at about 23° C. than that of LDEE.HCl, the salt formed of LDEE and HCl. The rate of hydrolysis at a particular pH and temperature can therefore depend on the buffering agent, e.g., citric acid and/or citrate anions, becoming faster when their concentration is elevated. Typically, the concentration of the buffering ions, i.e., the combined concentrations of citrates and citric acid, is between about 5 mM and 100 mM, for example between about 10 mM and 50 mM. The rate of hydrolysis can increase, for example, when the buffering citric acid-citrate concentration is increased.

Infusion of a Low pH LD-Prodrug Pharmaceutical Composition.

We have discovered that the incidence of adverse local effects can be reduced by making the infused pharmaceutical composition acidic. The pH of the infused solution is generally between 2.1 and 4.6; it is preferably between pH 2.6 and 4.2, for example 2.6±0.3, 2.8±0.3, 3.0±0.3, 3.2±0.3, 3.4±0.3 or 3.6±0.3; 3.9±0.3.

Hydrolytic Stability of the LD-Prodrug Pharmaceutical Compositions

Precipitation of LD produced upon hydrolysis of the prodrug can be retarded either by concentration or dilution of aqueous LD prodrug solutions. Dilution retards precipitation because in order to reach the solubility limit a greater fraction of the LD-prodrug must be hydrolyzed. For example, if at about neutral pH and at about 25° C. the solubility of LD would be about 0.025 M, i.e., if precipitation could not occur unless an LD concentration of 0.025 M would be exceeded, then at least 10% of the LD-prodrug in a 0.25 M solution would need to be hydrolyzed for precipitation to become possible; in a 0.5 M LD-prodrug solution precipitation could become thermodynamically possible already when 5% of the prodrug is hydrolyzed. Considering that the hydrolysis could be a first-order reaction, i.e., its rate could be proportional to the concentration of the prodrug, it could take twice as long for precipitation to become thermodynamically possible in the 0.25 M LD-prodrug solution than in the 0.5 M LD prodrug solution. High concentration can also retard precipitation, because in a sufficiently highly concentrated prodrug salt solution, for example LDEE.HCl solution of greater than about 2.5 M concentration, for example concentrations between about 2.5M and about 3.5 M, or higher than 3.5 M concentration, the concentration of reactive water can be low. In general, hydrolysis can be slow enough for absence of precipitation when the solution is stored at 5±3° C., for example at 4±1° C., for at least 6 months, for example for more than 12 months, when the LD-prodrug concentration, for example the LDEE.HCl concentration, is less than about 0.75 M or when it is higher than 2.5 M. Hydrolysis can also be slow in increasingly concentrated solutions, such as prodrug concentrations greater than 2.5 M, 3 M, or 3.5 M, but infusion of such concentrated solutions can increase the severity or frequency of infusion site tolerability issues. Solutions of concentrations between about 0.25 M and about 0.75 M LDEE.HCl can be infused without dilution by the patient or caregiver, facilitating their use by movement impaired PD patients.

The concentration of the LD-prodrug in the infused pharmaceutical composition can be between 0.15 M and 1.5 M, for example between 0.2 M and 1 M, or between 0.25 M and 0.75 M. The concentration of the LD-prodrug in the acidic, subcutaneously infused pharmaceutical composition can be high enough to allow the daily infusion of less than 40 mL, 30 mL, 25 mL, 20 mL, 18 mL, 16 mL, 15 mL, 14 mL, 13 mL, 12 mL, 10 mL, 9 mL, 8 mL, 7 mL, 6 mL, 5 mL, 4 mL, 3 mL, or 2 mL of the LD prodrug pharmaceutical composition.

Aqueous LD-prodrug solutions having long shelf lives, particularly refrigerated shelf lives, can differ in their compositions from those having good infusion site tolerability. For example, concentrated LDEE.HCl solutions, e.g., of greater than 2.6 M or 2.9 M concentration, can have refrigerated shelf lives of several years but they may be poorly tolerated at the infusion site. Some of the aqueous, acidic LD-prodrug solutions can be infused with few skin symptoms and can also have a shelf life of 3 months or longer, for example 6 months or longer, or 12 months or longer, or 18 months or longer, when stored refrigerated at a temperature of 5±3° C., e.g., near 4° C.

In the dry solid the rate of hydrolysis can be very slow. When a solid, dry LD-prodrug containing composition is stored at ambient temperature near 25° C., its shelf life can be 3 months or longer, for example 6 months or longer, or 12 months or longer, or 18 months or longer, or 24 months or longer. The solid LD-prodrug containing composition can be stored with refrigeration at about 5±3° C., for example at about 4±2° C., for more than 3 months, 6 months, 12 months, 18 months, 24 months, 36 months, or 48 months. The solid can be stored in one or more chamber of a container. The content of the solid LD prodrug containing chamber is dissolved in water, or in a pH buffering solution prior to use to provide the concentration and pH of the infused pharmaceutical composition.

The hydrolysis of a dissolved LDE can be slow in an aqueous pharmaceutical composition at an acidic pH and at a low temperature. The shelf life of an LDE increases as the pH is lowered from neutral to the range from about pH 6 to about pH 5, increases further when the pH is lowered to the range from about pH 5 to pH 4, increases further when it is lowered from about pH 4 to about pH 3, and can be particularly long at about pH 2.7±0.5, for example at about pH 2.7. The hydrolysis of LDEE.HCl, is correspondingly pH dependent. Hydrolysis can be very fast near neutral pH and can decrease as the pH decreases until about pH 2.4, then can increase below pH 2 as the pH is further decreased. It can be advantageously slow in the pH range between about 2.6 and 3.6, for example at pH 3.1±0.3 or at pH 3.3±0.3. Solutions of such pH can be infused and can stored for 3 months or longer, for example for 6 months or longer or 12 months or longer or 18 months or longer when refrigerated at a temperature of 5±3° C., e.g., near 4° C., without precipitation of hydrolytically formed LD.

The pH of a subcutaneously infused pharmaceutical composition can be generally greater than about 2.4. The preferred operational pH range can be between about 2.6 and about 4.6, e.g., between 2.8 and 4.2, for example 3.1±0.3 or 3.3±0.3, or 3.7±0.3. There would be no LD precipitation in an exemplary 1.0±0.5 M aqueous LDEE.HCl pharmaceutical composition or in an LDEE.HCl pharmaceutical composition of a concentration between 0.25 M and 0.75 M having a pH of 2.7±0.5 stored at about 5±3° C. (e.g., about 4° C.) for about more than a year. Upon raising the temperature to about 37° C. there would be no LD precipitation for more than 24 hours; or upon raising the temperature to about 30° C. for 48 hours.

Oxidative Stability of the LD-Prodrug Pharmaceutical Compositions

The LD prodrug (e.g., LDA or LDE) formulations of the invention can be designed to enhance stability by reducing the rates of their hydrolysis, which usually dominates their degradation. While the dominant degradation process in the presence of water is hydrolysis, the LD prodrugs can also be oxidized by dissolved or gaseous oxygen. The oxidation products can be less effective or ineffective prodrugs, and can reduce infusion site tolerability. In the absence of frequent monitoring (e.g., by HPLC or mass spectroscopy), oxidation makes accurate dosing difficult or impossible. The rate of oxidation can be reduced by several methods. One approach is to substantially exclude oxygen or reduce its partial pressure. The second is to include antioxidants, particularly pharmaceutically acceptable radical scavengers. The third is to maintain an acidic environment of a pH between about 2.3 and about 3.9, for example of about pH 3.5±0.4, 3.0±0.5, 2.8±0.3 or 2.5±0.3.

Doses, Solution Concentrations and Infused Volumes

The daily required amounts of LD for PD management are generally between about 1.5 millimoles and 15 millimoles, typically between about 2.5 and 10 millimoles, often near about 5 millimoles. At LD prodrug concentrations of >0.2 M, >0.3 M, >0.4 M, >0.5 M, >0.6 M, >0.8 M, and >1.0 M in the aqueous pharmaceutical compositions, the volumes can be small and can be administered subcutaneously. For example the infused volume in an advanced PD patient requiring daily as much as about 10 millimoles of the LD prodrug, i.e., daily about 2 g LD, the respective subcutaneously infused volume can be less than 50 mL, 33 mL, 25 mL, 20 mL, 16.7 mL, 12.5 mL, and 10 mL. Preferably the required volume is infused at multiple sites such that each site is infused with a lesser volume and dose. For example, infusion at 4 sites reduces the daily dose at a site to 2.5 millimoles and the volumes infused at a site respectively to less than 12.5 mL, 8.2 mL, 6.3 mL, 5 mL, 4.2 mL, 3.2 mL, and 2.5 mL.

Viscosity of the Pharmaceutical Compositions

The preferred subcutaneously infused aqueous pharmaceutical compositions including an LD prodrug (e.g., LDA or LDE) can have viscosities of less than about 10⁴ centipoise, preferably less than about 10³ centipoise, preferably between about 1.2 cp and about 2×10² cp at about 25° C. measured, for example, with a glass capillary viscometer or by a falling sphere viscometer. The viscosity of the infusible LD prodrug (e.g., LDA or LDE) compositions can typically be between about 1.2 cP and about 200 cp (e.g., between about 2 cp and 50 cP), when the viscosity is measured by glass capillary (Oswald) viscometer, or a falling sphere viscometer, or by a Brookfield viscometer, such as model LVDV-E of Brookfield Engineering Laboratories (11 Commerce Boulevard, Middleboro, Mass. 02346-1031 USA).

Crystallization Inhibitors

Adsorption of macromolecules on growing faces of crystallites prevents or reduces access of molecules of the precipitated solute, often fully preventing, or slowing, growth of the crystallites to dimensions where their surface/volume ratio is high enough for thermodynamic stability, the high surface energy de-stabilizing small crystallites (i.e., slowing the rate of nucleation or preventing nucleation). The aqueous liquid formulations of the invention can include the LD prodrug (e.g., LDA or LDE) formulated with one or more crystallization inhibitors, such as a sugar (e.g., hydroxyethyl starch, dextran, albumin, polyethylene glycol, mannitol, glucose), hyaluronic acid, succinylated gelatin, or other polycarboxylic acids.

Soluble Co-Infused DDC Inhibitors

The invention also features formulations including an acid addition salt of benserazide like benserazide.HCl, or a benserazide prodrug, or a carbidopa prodrug (e.g., an acid addition salt of a carbidopa ester or acid addition salt of a carbidopa amide, such as a carbidopa ester hydrochloride or a carbidopa amide hydrochloride), which are adequately soluble in water, which can be stable in pharmaceutical composition, which can be delivered via an ambulatory infusion pump, and which can increase the LD half-life in the PD patient and/or reduce the daily LD or LD prodrug dose. The soluble DDC inhibitor or its prodrug can be optionally co-dissolved and/or co-infused with the LD-prodrug. When co-infused with the LD prodrug the benserazide or carbidopa prodrug can reduce the total daily infused LD prodrug dose. The carbidopa prodrug can be formulated to prevent rapid hydrolysis prior to its infusion, yet to be rapidly hydrolyzed to form carbidopa after its delivery into the body. Preferred carbidopa esters are rapidly hydrolyzed in vivo by esterases and preferred carbidopa amides are rapidly hydrolyzed in vivo by amidases.

The hydrochloride salt of benserazide is water soluble. The solubilities of carbidopa prodrugs like carbidopa esters and carbidopa amides usually exceed the solubility of carbidopa, the highest solubilities typically being observed for carbidopa prodrug salt forms. For example, the solubilities of salts of carbidopa ethyl ester and carbidopa methyl ester, such as the salts formed when these bases are neutralized by HCl, are much more soluble than carbidopa. For example, the high solubility of carbidopa ethyl ester hydrochloride allows for aqueous pharmaceutical compositions of high concentration. The carbidopa prodrugs are hydrolyzed to carbidopa, which can be much less soluble in water or in aqueous pharmaceutical compositions in the pH range suitable for subcutaneous infusion.

The prodrugs of the invention can be stored in liquid forms or solid forms, which can provide upon mixing of the contents of two containers or chambers an infusible aqueous pharmaceutical composition prior to infusion into a subject. The shelf life of the stored and of the infused, e.g., subcutaneously infused, carbidopa prodrug pharmaceutical compositions is usually determined by their hydrolysis. Their hydrolysis leads to carbidopa precipitation, which can be faster than other degradation processes, such as oxidation, particularly in acidic pharmaceutical compositions and/or when oxygen is substantially excluded. For this reason, a major problem with the carbidopa prodrug-containing formulations, particularly of aqueous formulations, is their hydrolytic instability. The rate of hydrolysis is pH and temperature dependent. Because the carbidopa is poorly soluble, and because the concentration of the carbidopa prodrug in the small-volume subcutaneously infused pharmaceutical composition is high, even hydrolysis of a fraction of a carbidopa prodrug or prodrug salt may result in the precipitation of carbidopa from the pharmaceutical composition. The presence of a large amount of carbidopa precipitate is unacceptable, as it may lead to a dosing error and because it may block or reduce the flow in the infusion system.

At a particular temperature and pH, the carbidopa esters formed of carbidopa and of different alcohols are hydrolyzed at different rates. For example, it is expected that the rate of hydrolysis of carbidopa methyl ester could be slower than the rate of hydrolysis of carbidopa ethyl ester. The rate of hydrolysis of carbidopa ester salts could also depend on the anion, i.e., on the acid forming the carbidopa ester salt. The hydrolysis of carbidopa ester salts, such as carbidopa ethyl ester hydrochloride, is expected to be strongly pH dependent. It is expected to be fast near neutral pH; to decrease as the pH decreases until about pH 2.5; below about pH 1 it is expected to increase as the pH is further decreased. In strongly acidic pharmaceutical compositions, e.g., of about pH 0.5 or less, the expected rate of hydrolysis is even faster.

Although precipitation of carbidopa can be retarded or prevented by diluting the concentrated prodrug pharmaceutical composition, excessive dilution defeats administration in the small volume preferred for subcutaneous administration. The shelf life can be very short for the typical carbidopa prodrug aqueous pharmaceutical composition at about neutral pH at an ambient temperature (e.g., 25° C.). To minimize hydrolysis and carbidopa precipitation, the carbidopa ester salt can be stored in its dry solid form, and dissolved in water or in an aqueous pharmaceutical composition prior to use. Alternatively, the carbidopa ester salt can be dissolved, and stored as an aqueous pharmaceutical composition at a pH and at a temperature where the rate of hydrolysis is slow. The shelf life is expected to increase as the pH is lowered from neutral to the range from about pH 5 to pH 4, increase further when it is lowered from about pH 4 to about pH 3, and can be particularly long at about pH 2.7±1.0, for example at about pH 2.8±0.3, or pH 3.1±0.3 or pH 3.3±0.3. The operational life is similarly pH dependent. The pH of a subcutaneously infused pharmaceutical composition can be generally greater than about 2.0. The preferred operational pH range is between about 2.0 and about 4.2, the range between about 2.2 and 3.9 being more preferred; for example the pH of the subcutaneously infused pharmaceutical composition can be 3.5±0.4, 3.0±0.5 or 2.5±0.3. To extend the shelf life, the pharmaceutical composition may be optionally stored at a temperature below about 25° C., for example it may be refrigerated at about 5±3° C. No carbidopa precipitation is expected in an exemplary 1.0±0.5 M aqueous carbidopa ethyl ester hydrochloride pharmaceutical composition when having a pH between about 2.4 and about 3.5 and stored at about 5±3° C. (e.g., about 4° C.) for more than 1 year, e.g., when buffered to have a pH of 2.8±0.3 and stored at about 5±3° C. (e.g., about 4° C.). Upon raising the pH of the pharmaceutical composition after 18 months of refrigerated storage to about 3.0±1.0 it would still remain precipitate free after more than 2 days at an operational temperature of 37° C., and for more than about 3 days at an operational temperature of 30° C.

The daily required amounts of benserazide or carbidopa for PD management are generally between about 0.3 millimoles and 5 millimoles, typically between about 0.6 and 2.5 millimoles, and most often of about 1-2.5 millimoles. At concentrations of >0.2, >0.3 M, >0.4 M, >0.5 M in aqueous pharmaceutical compositions, the volumes can be small and can be infused subcutaneously. Carbidopa has a longer in-vivo, e.g., plasma half-life than LD and large daily doses are well tolerated. Consequently, part of the DDC inhibitor could be delivered orally, by inhalation, or transdermally, and the infused dose could be thereby reduced.

Antioxidants

LD and LD prodrugs (e.g., LDA or LDE) can be susceptible to oxidative degradation. To minimize oxidative degradation the formulations of the invention optionally contain one or more antioxidants. Antioxidants that can be used in the aqueous formulations of the invention can be selected from thiols (e.g., dihydrolipoic acid, propylthiouracil, thioredoxin, glutathione, cysteine, cystine, cystamine, thiodipropionic acid), sulphoximines (e.g., buthionine-sulphoximines, homo-cysteine-sulphoximine, buthionine-sulphones, and penta-, hexa- and heptathionine-sulphoximine), metal chelators (e.g, α-hydroxy-fatty acids, lactoferrin, citric acid, lactic acid, and malic acid, EDTA, EGTA, and DTPA); or reducing agents, such as sodium metabisulfite, vitamin C, sodium ascorbate, magnesium ascorbyl phosphate, and ascorbyl acetate, phenols, uric acid, or combinations thereof. The total amount of antioxidant included in the formulations can be from 0.01% to 2% by weight.

LD Prodrugs

The invention features compositions, methods, and infusion pumps for infusing an LD prodrug and/or its salt. Typically, the proton of the primary amine of th LD-prodrug is pronated in the infused acidic solution meaning that the infused prodrug in the aqueous acidic solution is mostly in its ammonium cation form The LDEs are hydrolyzed in vivo to an alcohol and LD; the LDAs are hydrolyzed in vivo to LD and an ammonium salt, mostly an ammonium chloride. In general, the oral, i.e., ingested LD₅₀ of the produced alcohol, or ammonium chloride is greater than 3 millimoles/kg.

LDEE can be prepared from LD and ethanol, for example, as described in PCT Publication Nos. WO2003/042136 and WO2000027801; as described in U.S. Pat. Nos. 5,525,631; 6,218,566, and/or 5,354,885; or as described by Marrel et al., European Journal of Medicinal Chemistry, 20:459 (1985), each of which is incorporated herein by reference. Other esters of LD can be prepared from LD and the corresponding alcohol using analogous synthetic methods.

In aqueous LDE salt pharmaceutical compositions, the hydrolysis rates generally decrease as the pH decreases, and the shelf life of the LDE salt consequently increases, unless the pH is about 1.7±0.5 or less. Thus at about pH 2.5 the LDEE.HCl pharmaceutical compositions are generally more stable than at about pH 3.5; at pH 3.5 the LDEE.HCl pharmaceutical composition are generally more stable than at about pH 4.5; at about pH 4.5 they are generally more stable than at about pH 5; at about pH 5 they are generally more stable than at about pH 6; and at about pH 6 they are generally more stable than they are at about pH 7. In acidic pharmaceutical compositions the amines of the LDEs are protonated, making the LDEs cations. At neutral pH, LDEE is hydrolyzed within hours or less, making the pH 7 pharmaceutical composition unsuitable for most infusion situations. The rates of hydrolysis generally increase with temperature, and may at least about double or about triple for each 10° C. increase, correspondingly decreasing upon cooling.

The infused pharmaceutical compositions may include LDA and/or LDE. The LDAs can be synthesized using the methods described by Zhou et al., European Journal of Medicinal Chemistry, 45:4035 (2010).

LD prodrugs can be prepared from LD in a process that may include the selective protection and deprotection of the hydroxyl, amine, and/or carboxyl functional groups of the LD. For example, commonly used protecting groups for amines include carbamates, such as tert-butyl, benzyl, 2,2,2-trichloroethyl, 2-trimethylsilylethyl, 9-fluorenylmethyl, allyl, and m-nitrophenyl. Other commonly used protecting groups for amines include amides, such as formamides, acetamides, trifluoroacetamides, sulfonamides, trifluoromethanesulfonyl amides, trimethylsilylethanesulfonamides, and tert-butylsulfonyl amides. Examples of commonly used protecting groups for carboxyls include esters, such as methyl, ethyl, tert-butyl, 9-fluorenylmethyl, 2-(trimethylsilyl) ethoxy methyl, benzyl, diphenylmethyl, o-nitrobenzyl ortho-esters, and halo-esters. Examples of commonly used protecting groups for hydroxyl groups include ethers, such as methyl, methoxymethyl, methoxyethoxymethyl, methylthiomethyl, benzyloxymethyl, tetrahydropyranyl, ethoxyethyl, benzyl, 2-napthylmethyl, o-nitrobenzyl, p-nitrobenzyl, p-methoxybenzyl, 9-phenylxanthyl, trityl (including methoxy-trityls), and silyl ethers. Protecting groups can be chosen such that selective conditions (e.g., basic conditions, catalysis by a nucleophile, catalysis by a Lewis acid, or hydrogenation) are required to remove each, exclusive of other protecting groups in a molecule. The conditions required for the addition of protecting groups to amine, hydroxyl, and carboxyl functionalities and the conditions required for their removal are provided in detail in T.W. Green and P.G.M. Wuts, Protective Groups in Organic Synthesis (2nd Ed.), John Wiley & Sons, 1991 and P.J. Kocienski, Protecting Groups, Georg Thieme Verlag, 1994.

LD-treated people with advanced PD require typically daily 0.5-3 g (2.5-15 millimoles) oral LD. The prodrug can be subcutaneously infused typically in a daily volume of less than 40 mL, 35 mL, 30 mL, 25 mL, 20 mL, 18 mL, 16 mL, 15 mL, 14 mL, 13 mL, 12 mL, 11 mL, 10 mL, 9 mL, 8 mL, 7 mL, 6 mL, or 5 mL.

The preferred anion of the LDE or the LDA is the chloride ion, the only anion present in body fluids at >0.1 M concentration, because infusion of 5 or more millimoles of its salt does not substantially affect its systemic concentration. For this reason, the preferred anion is chloride, i.e., in the case of LDEE the preferred salt is LDEE.HCl.

The LD prodrug (e.g., LDE or LDA) may be administered in its free base form or as a pharmaceutically acceptable salt, preferably its chloride salt, i.e., the hydrochloride salt formed of the free base and HCl. It may be administered also as a salt with an anion known to be very rapidly metabolized through cycles, such as the Krebs cycle (e.g., citrate, gluconate, malate, malonate, fumarate, succinate, isocitrate, or 1-glycerophosphate). In certain instances the formulation of the invention includes a hydrochloride salt.

LDEE.HCl and the Stabilization of its Aqueous Pharmaceutical Compositions

The hydrolytic stability of concentrated aqueous pharmaceutical compositions of LDEE.HCl is best between about pH 2.0 and about pH 3, and it is preferred to store the pharmaceutical compositions at pH 2.7±0.5. Such a pH can be maintained for example through co-dissolving citric acid and/or a citrate salt, e.g., monosodium citrate, for example to about 10-50 mM concentration. For infusion it is desired, in order to avoid acid-caused pain, to raise the pH at least to pH 2.4±0.3, for example to 2.4±0.3, 2.6±0.3, 2.8±0.3, 3.1±0.3, 3.4±0.3, 3.7±0.3 or to 4.0±0.3, for example by adding citric acid, monosodium citrate, or trisodium citrate. Exemplary estimated storage and operational lives are provided in Table 1 for an about 1.0 M LDEE.HCl infusible pharmaceutical composition initially of about pH 3.7±0.2.

TABLE 1

| | |
|---|---|
| Infused volume (for delivery of 1 g LD equivalent), mL | 5 |
| Estimated storage life at 4° C., months | >6 |
| Estimated storage life at 25° C., weeks | >2 |
| Estimated operational life at 30° C., days | >2 |
| Estimated operational life at 37° C., days | >1 |

Exemplary estimated storage and operational lives are provided for an about 0.44 M LDEE.HCl infusible pharmaceutical composition initially of about pH 3.7±0.2 in Table 2.

TABLE 2

| | |
|---|---|
| Infused volume (for delivery of 1 g LD equivalent), mL | 11.3 |
| Estimated storage life at 4° C., years | >1.5 |
| Estimated storage life at 25° C., months | >1 |
| Estimated operational life at 30° C., days | >3 |
| Estimated operational life at 37° C., days | >2 |

Infusion Pumps and Pulsed Dosing

The invention also features infusion pump systems for the administration of the formulations and methods of infusion.

The pharmaceutical compositions of the invention, optionally in combinations with other drugs used for the treatment of PD, such as enzyme inhibitors like benserazide.HCl or a soluble prodrug of carbidopa (such as its ethyl ester hydrochloride or its methyl ester hydrochloride), can be infused subcutaneously using an infusion pump, which can optionally be a syringe-type infusion pump. The pump can be configured to automatically infuse continuously or intermittently, and/or administration can be subject-controlled. For example, the pump can be configured to administer a pharmaceutical composition of LD prodrug (e.g., a LDE, such as LDEE) intermittently.

In pulsed dosing, a solution of a Parkinson's disease treating drug is infused at a high flow rate at one site or at a group of sites during a brief infusion period, then the flow is reduced, optionally to nil, during a non-infusion period that is longer the period of the high flow rate. Typically it is preferred to sustain the high flow rate for 20 minutes or less, for example for less than about 15 minutes, 10 minutes, or 5 minutes. The rest period can be 15-20 minutes, 20-25 minutes, 25-30 minutes, 30-40 minutes, 40-50 minutes, 50-60 minutes, 60-90 minutes, or 90-120 minutes.

Any suitable type of infusion pump may be used to deliver the LD prodrug (e.g., LDA or LDE) liquid composition. These may include implantable and non-implantable pumps, pumps for intramuscular, subcutaneous, percutaneous, or intrathecal delivery, fixed position or ambulatory pumps, patch pumps and carried pumps. These pumps may employ any pump drive mechanism known in the art including syringe, hydraulic, gear, rotary vane, screw, bent axis, axial piston, radial piston, peristaltic, spring-driven, gas-driven, piezo-electric, electroosmotic, and wax expansion. For example, for infusing large volumes, an infusion pump can include a peristaltic pump. Alternatively, for infusing small volumes, an infusion pump can include a computer-controlled motor, turning a screw that pushes the plunger of a syringe.

Ambulatory drug infusion pumps can be used for subcutaneous or intravenous administration of a pharmaceutical composition of the invention. One example of an ambulatory infusion pump used to treat PD is the Smiths Medical CADD-Legacy 1400 ambulatory pump, which is used to deliver the Duodopa gel. The pump is reusable and works with a disposable cassette containing the drug. The cassette has a 100 mL reservoir containing 20 mg/mL LD and 5 mg/mL carbidopa in a gel; carmellose sodium is used as a thickening agent. The shelf life is 15 weeks when refrigerated, and 24 hours at room temperature. The Duodopa gel is infused from the extracorporeal pump to the duodenum through a catheter that is surgically implanted through the wall of the abdomen in a percutaneous gastrostomy operation.

Some features of the CADD-Legacy pump include a display, cassette detection, occlusion detection, air-in-line detection, on/off key, event memory and programmable infusion rates. The infusion regimen suggested in the Duodopa users guide includes a morning dose (administered when the subject wakes up in order to quickly achieve the concentration required for optimal subject response); a continuous maintenance dose (administered continuously by the pump to maintain a constant circulating concentration); and extra doses (administered if the subject experiences reduced mobility during the day).

Another example of an ambulatory infusion pump is the Crono APO-go pump (Canè s.r.l. Medical Technology Via Pavia 105/I Rivoli (TO) Italy) for infusion of apomorphine, a dopamine agonist. It is indicated for the treatment of disabling motor fluctuations (“on-off” phenomena) in subjects with PD. The pump infuses apomorphine 10 mg/mL pharmaceutical composition.

A particular class of ambulatory drug infusion pumps, which can be used for the delivery of the pharmaceutical compositions of the invention, are pumps designed to infuse drugs, for example insulin to patients with diabetes. These can generally be broken down into two groups: skin-attached “patch pumps” and carried pumps. Examples of insulin patch pump designs by various companies include those described in U.S. Pat. Nos. 7,914,499, 7,806,867; 7,740,607; 7,530,968; 7,481,792; 7,771,412; 7,303,549; 7,144,384; 7,137,964; 7,029,455; 7,018,360; 6,960,192; 6,830,558; 6,768,425; 6,749,587; 6,740,059; 6,723,072; 6,699,218; 6,692,457; 6,669,669; 6,656,159; 6,656,158; 6,485,461; 7,815,609; 7,771,391; 7,713,262; 7,713,258; 7,632,247; 7,520,295; 7,517,335; 6,726,655; 6,669,668; 6,428,518; 6,416,496; 6,146,360; and 6,074,366, and U.S. Patent Publication Nos. 20110137287, 20100217191; 20100274218; 20100243099; 20080319416; 20080319414; 20080319394; 20080319384; 20080255516; 20080234630; 20080215035; 20070191702; 20100137784; 20070250007; 20060206054; and 20090320945, each of which is incorporated herein by reference. Examples of carried pump designs by various companies include those described in U.S. Pat. Nos. 6,551,276 and 6,423,035, each of which is incorporated herein by reference. The preferred pumps are inexpensive, optionally single-use, skin attached patch pumps, optionally with two compartments. One, two or more inexpensive patch-pumps can be attached to the skin in order to increase the dose rate or the dose of the LD-prodrug, or to better distribute the infused volume.

An exemplary useful pump is the Crono syringe-type programmable infusion pump of Canè s.r.l. Medical Technology Via Pavia 105/I Rivoli (TO) Italy. Its dimensions are 77×48×29 mm (3×1.9×1.1 inch) and its weight is 115 g, its 3 Volt type 123 A lithium battery is included. The capacity of its syringe is 10 or 20 mL. The delivered pharmaceutical composition volume can be programmed from 1 to 20 mL for delivery times from 30 minutes to 99 hours, in 15 minutes steps. The accuracy is +/−2%. The occlusion pressure is 4.5±1 bar. The pump is programmable and the data are automatically stored in the pump's memory; in the event of an anomaly, an alarm is provided and an error message is displayed. The pump's functions can be “locked” such that the subject will not accidentally change a function by pushing a button. The pump operates accurately in the 10° C.-45° C., at 30%-75% relative humidity and through the 700 hPa-1060 hPa (hectopascal) atmospheric pressure range.

Yet another exemplary useful pump that can be used in the methods and devices of the invention is an electro-osmotic drug pump, such as that described in PCT Publication No. WO 2011112723; W. Shin et al., Drug Delivery and Translational Research 1:342 (2011); W. Shin et al., Journal of the American Chemical Society 133, 2374 (2011); and in W. Shin et al., Analytical Chemistry 83(12), 5023 (2011); Nagarale et al. Journal of the Electrochemical Society 159(1), 14 (2012).

The pumps preferred are externally worn and infuse subcutaneously and can infuse pharmaceutical compositions of 1 cP, 10 cP, 100 cP, 1000 cP viscosity at about 30° C. at average rates of more than 1 μL per min, preferably at least 2, 5, 10 μL per minutes.

Infusions may be made continuously or intermittently, with sample intermittent infusion intervals being less than or equal to about every 5, 10, 15, 30, 60, 90 or 120 minutes.

Infusion rates may be set to one or more values that equates to a rate of LD prodrug (e.g., LDA or LDE) delivery of anywhere between 1-300 mg/hr. For subcutaneous infusion representative rates may be between 10-200 mg/hr. Sample infusion rates may equate to about 10, 20, 30, 40, 50, 60, 70, 80, 90, 100, 125, 150, 175, 200, 225, 250, 275, 300, or 325 mg/hr of LD prodrug (e.g., LDA or LDE).

Pump flow rates depend on the concentration of the LD prodrug (e.g., LDA or LDE) in the pharmaceutical composition and on the duty cycle, which is the delivery/(delivery+non-delivery) time ratio. Convenient time averaged flow rates are less than or equal to about 1.4, 1.2, 1.0, 0.8, 0.6, 0.4, 0.3, 0.2, 0.1 or 0.04 mL/hr. During delivery periods of pulsed operation the flow rates are generally these values multiplied by the non-delivery/delivery ratio, which can be 12, for example, when the delivery time is 5 min and the non-delivery time is 55 min.

LD prodrug (e.g., LDA or LDE) from a single container may be infused s.c. by the pump for a period of greater than or equal to about 8 hours, 12 hours, 16 hours, 24 hours, 48 hours, 72 hours or 96 hours. The container may contain the equivalent of between 0.25-20 g of LD prodrug (e.g., LDA or LDE), or of 1-6 g of LD prodrug (e.g., LDA or LDE). Examples of the equivalent amounts of LD prodrug (e.g., LDA or LDE) that may be contained in a container are about 0.5±0.2, 1±0.3, 2±0.4, 3±0.5, 4±0.7, 5±0.8, 6±2, 8±2, 10±2, 12±2, 14±2, 16±2, 18±2, or 20±2 g. Implantable pumps may contain greater amounts of drug in their reservoirs.

Any suitable type of infusion pump may be used to subcutaneously deliver the compositions. These may include implantable and non-implantable pumps, fixed position or ambulatory pumps, patch pumps and carried pumps. The pumps preferred are externally worn and infuse subcutaneously and can infuse pharmaceutical compositions of 1 cP, 10 cP, 100 cP, 1000 cP viscosity at about 30° C. at average rates of more than 0.1 mL/hr, preferably at least 0.2 mL/hr, 0.3 mL/hr, 0.4 mL/hr, 0.5 mL/hr, or 0.6 mL/hr. Typical infused LD prodrug dose ranges are from about 10 micromoles per kg of subject weight to about 250 micromoles per kg of subject weight of LD prodrug per day. For example, the typical daily dose for a subject weighing 75 kg is from about 0.75 millimoles to about 15 millimoles of LD prodrug. Infusion rates may be set to one or more values that equates to a rate of LD prodrug delivery of anywhere between about 30 micromoles per hour and about 1,000 micromoles per hour. For an exemplary pharmaceutical composition in which the concentration of the LD prodrug is 0.5 M, these values correspond to average flow rates of 60 and 2,000 microliters per hour respectively. The exemplary dosage/kg of LD prodrug to be administered is likely to depend on such variables as the stage of the PD of the subject, the dose/kg being higher for subjects in more advanced stages of the disease and on the particular formulation of the LD prodrug being used. It is also likely to depend on the age of the subject, likely higher for subjects younger than about 60 and lower for subjects older than about 60. In continuous operation, the preferred pump time averaged flow rate can be between about 0.1 mL per hour and about 3.0 mL per hour. In intermittent operation the flow rate depends on the duty cycle. For example, if the pump is on for 10 minutes and is off for 20 minutes the pumping rate while the pump is on is between 0.6 mL per hour and about 7.5 mL per hour. LD prodrug from a container may be infused s.c. by the pump for a period of greater than or equal to about 8 hours, 12 hours, 16 hours, 24 hours, 48 hours, 72 hours or 96 hours. The container may contain between about 1 millimole and about 100 millimoles of LD prodrug. An about 20-35 mL exemplary container, typically replaced daily, may contain about 15 millimoles of the LD prodrug, enough to yield after its hydrolysis in the body about 3 g LD.

In one embodiment the flow rate is constant rather than being adjusted by the user or health care provider. Pumps with different constant flow rates are to be provided for users requiring different daily doses of the LD prodrug. In another embodiment the flow rate is constant for all users, and the users are provided with pharmaceutical compositions of different LD-prodrug concentrations. Advantages of the fixed flow rate pumps include their low cost and the simplicity of their use.

Pumps of the present invention can include some or all of the following elements: a pump drive mechanism; a subcutaneous infusion set, at least one cannula or needle; at least one inserter for the subcutaneous infusion set, cannula, or needle; a drug reservoir; a display; an input mechanism (e.g., a keypad or touchscreen); a memory; a remote control; a data processing unit; an alarm; a battery; a timer; an actuator; a transmitter; a receiver; an occlusion sensor; data download or transmission capability; the ability to input disease-related data (e.g., event markers, sensor measurements, meals, exercise, etc.); algorithms to recommend or control drug basal and/or bolus dosing; and an adhesive to attach to the skin or a clip to attach to clothes. The pumps can be configured to obtain data from sensors through a physical or a wireless connection, or even from physical integration of the units. The pump may also be configured to communicate with tremor or movement monitoring accelerometers, computers, cell phones, the internet and various communication networks.

The reservoir of the pump can be a graduated or non-graduated glass or plastic syringe prefilled with the LD-prodrug pharmaceutical composition; on one end the cylinder can be attached onto an infusion pump, such as the Crono pump; on the opposite side the cylinder can be attached to the infusion set. The attachment to the infusion set can be, for example, the male part or the female part of a luer lock. The plastic of the cylinder can be made from a polymer having low oxygen permeability, such as polyvinylidene chloride, filler loaded butyl rubber (poly(isobutylene-co-isoprene)), filler loaded chlorobutyl rubber, chlorobutyl rubber, bromobutyl rubber, butyl rubber, chlorosulfonated polyethylene (Hypalon), or amorphous polyethylene terephthalate.

Alternatively the drug reservoir(s) can be equipped with a septum, which is penetrated to provide fluid contact between the reservoir and a needle, e.g., of the infusion set. The septum of the reservoir can be made from a polymer having low oxygen permeability, such as polyvinylidene chloride, filler loaded butyl rubber (poly(isobutylene-co-isoprene)), filler loaded chlorobutyl rubber, chlorobutyl rubber, bromobutyl rubber, butyl rubber, chlorosulfonated polyethylene (Hypalon), or amorphous polyethylene terephthalate.

The infusion set can be single pronged or it can be multi-pronged, e.g., bifurcated, trifurcated, quadrifurcated or multifurcated. The infusion set's catheter(s), can be constructed to have low permeability to oxygen. The catheter can, optionally, be long, e.g. 60 cm; its ID can be typically less than 1 mm (e.g., about 0.7 mm, about 0.4 mm, or less). Its wall thickness can be less than 1 mm and greater than about 0.2 mm (e.g., between about 0.4 and about 0.6 mm). The catheter can be optionally formed from a polymer, such as polyvinylidene chloride, filler loaded butyl rubber (poly(isobutylene-co-isoprene)), filler loaded chlorobutyl rubber, chlorobutyl rubber, bromobutyl rubber, butyl rubber, chlorosulfonated polyethylene (Hypalon), or amorphous polyethylene terephthalate.

Simple, low cost, optionally pre-filled, pumps may be entirely or partially disposable after use, i.e a part of the pump or the entire pump may be exchanged after use. Non-programmable pumps may simply deliver a constant basal infusion rate; optionally, they may also have the ability to deliver a fixed bolus or multiple fixed boli on command.

The pump can include the software, memory and hardware to enable the pump to input, store, recall, display, communicate and/or analyze event markers useful to management of PD. Such event markers can include: (i) intake of the infused medications, including dose and time; (ii) intake of other PD medications, including identification of the drug, dose and time (e.g., such medications may include DDC inhibitors, dopamine receptor agonists, MAO-B agonists, COMT enzyme inhibitors, anticholinergics, amantadine, and/or other drugs); (iii) symptoms and side effects (e.g., on and off times, dose failures, delayed time to on, tremor, dystonia, akinesia, bradykinesia, dyskinesia, tremor, nausea, vomiting, confusion, somnolence, hallucination, insomnia, constipation, dizziness, dysphagia, moods and mood changes, and impulse control disorders); (iv) sensor readings or data; (v) sleep times and/or sleep quality; (vi) meals and meal information, particularly of the protein content of the meal; (vii) defecation information; (viii) and/or exercise information. Such event markers can also record the time of the event and additional information or notes specific to each event, such as its intensity, quality, duration, amount, or character, among other information.

The pump may be programmed to increase the amount of drug infused after it has been de-activated, for example for a sleeping period; or following meals that contain proteins, after which the blood concentration of neutral amino acids competing with LD for active transport across the blood brain barrier increases.

The pump may be used to infuse the LD prodrug over the entire 24-hour day. Alternatively, in order to reduce the possibility of side effects (e.g., hallucinations) from 24 hour infusion of LD, some physicians may prefer that the pump only infuse the LD prodrug about 12, 14, 16, 18, or 20 hours per day. When the subject goes to bed at night, the infusion may be stopped or reduced significantly, i.e., reduced to less than 50% of the average daytime infusion rate. After waking, the subject may initiate infusion at the regular basal rate or, if the subject is in the off state, at a higher “morning dose” rate, in order to turn on more quickly. The pump can be programmed to begin such morning infusions automatically so that the subject does not need to initiate them. For example, the pump may be programmed to initiate infusion at the regular basal rate or at a higher morning dose rate at a certain hour or a certain amount of time (e.g., 4, 6, or 8 hours) after the infusion was stopped or decreased. If the pump is programmed to initiate such an infusion before the subject typically gets up in the morning, then the subject can get up in the on state rather than in the off state. A morning dose rate is an infusion rate that is 10%, 20%, 30%, 40% or 50% greater than the basal rate or the average daytime infusion rate. When a fixed flow rate pump is used the subject may take an oral morning dose to turn on more quickly.

It may be difficult for a person with PD to input information or commands into the pump due to tremor or dyskinesia. Multistep inputs and those requiring fine motor skills (e.g., navigating through multiple menus on a screen, or using a keypad or a thumbwheel) may be particularly difficult. Consequently, a particularly useful means of providing input to the pump is to have one, two, three, four or more large, dedicated actuators on the pump or a remote control for the subject to easily activate in order to input frequently used or critical functions or information. Examples of such an actuator are one, two, three, four or more large buttons or switches that may be placed on the exterior of the pump or remote control. These buttons or switches may be of any convenient size. Examples include the range of 0.1 to 2.0 inches, or the range of 0.25 to 1 inch. Examples of frequently used or critical functions or information may include: deliver bolus; reduce infusion rate; increase infusion rate; or experiencing one or more of dyskinesia, bradykinesia, tremor, off state or on state. Specific examples are: a button to indicate dyskinesia; a button to indicate bradykinesia; a button to indicate rigidity; a button to indicate off state; a button to indicate akinesia; or a button to initiate a bolus. When a fixed flow rate skin-adhered pump is used the flow would start e.g., upon its application to the skin.

The pump can be integrated with a sensor to form a sensor-augmented pump. The pump system can include the software, memory and hardware to enable the pump to input, store, recall, display, communicate and/or analyze sensor data useful to management of PD. Such integration may be physical, in which case the sensor and the pump share some physical components (e.g., a housing, remote control, memory, a display, a power source). Alternatively, such integration may be through data communication in which case the sensor transmits data to the pump, the pump transmits data to the sensor, or both. The sensor can include a transmitter and/or a receiver. The sensor can be a unitary device or may be a system having physically separate components, such as a physically separate sensor component and a display, memory, data communication, analysis or other component. The sensor can be reusable or disposable.

Sensors of the present invention can include any physiological, physical or chemical parameter associated with the subject. Specific examples of sensors and sensed parameters include: (i) motion sensors (e.g., accelerometers to sense movement, stillness, slowness, falling, walking, akinesia, bradykinesia, tremor, restless leg, finger movement and/or leg movement; (ii) the accelerometers may also sense posture, such as whether the subject is standing, sitting or lying down); (iii) pressure transducers or electrodes to sense cardiovascular parameters (e.g., heart rate, electrocardiogram, etc.); (iv) electrodes to sense wakefulness or sleep, and sleep parameters (these may include polysomnography, electroencephalogram, electro-oculogram, and/or electromyogram); (v) pressure sensors to measure blood pressure; (vi) acoustical or electrical sensors to detect snoring and/or sleep apnea; (vii) chemical sensors to test blood, saliva or other body fluids for the presence or concentration of specific medications or analytes (e.g., LD, other PD medications, coumadin, glucose, etc.); (viii) and a sensor to detect the subject's location, for example using input from a global positioning system or local computer or cell phone networks. An example of an accelerometer that can be used in the pump systems of the invention is the Chronos eZ430 wireless watch sold by Texas Instruments.

The pump system can include hardware, software and algorithms that enable the system to recognize a situation and recommend to the subject a one-time adjustment to the drug delivery regimen, e.g., to take a bolus of LD prodrug (e.g., LDA or LDE) optionally combined with benserazide or a carbidopa prodrug (e.g., carbidopa ester or carbidopa amide). The pump system can include hardware, software and algorithms that enable the system to recognize patterns and recommend to the subject changes in his drug delivery regimen. The system can utilize for this purpose data from the stored event markers and data from sensors. The changes may be to the regimen of the drug being infused by the pump or to the regimen of other PD drugs being taken by the subject. For example: (i) if the system determines from user or sensor input that a subject has gone to bed or gone to sleep in the evening it may decrease the LD prodrug (e.g., LDA or LDE) infusion rate or stop the infusion altogether; (ii) if the system determines from user or sensor input that a subject has gotten out of bed or woken up in the morning it may provide a bolus of LD prodrug (e.g., LDA or LDE), increase the LD prodrug (e.g., LDA or LDE) infusion rate, or if the pump infusion had been stopped it may turn the pump infusion back on; (iii) if the subject has frequent or extended off periods then the system may recommend a revised drug infusion regimen with an increase in the LD prodrug (e.g., LDA or LDE) basal infusion rate; (iv) if the subject takes a long time to turn on after being off then the system may recommend a revised drug infusion regimen with an increase in the LD prodrug (e.g., LDA or LDE) bolus amount; (v) if the subject suffers dyskinesia, nausea or hallucination the system may recommend a revised drug infusion regimen with a decrease in the LD prodrug (e.g., LDA or LDE) basal infusion rate; (vi) if the subject suffers dyskinesia, nausea or hallucination the system may recommend that a scheduled LD prodrug (e.g., LDA or LDE) bolus be skipped or reduced; (vii) if user or sensor input indicates that the subject is suffering from akinesia the system may recommend that a one-time bolus be provided; (viii) if user or sensor input identifies a tremor the system may recommend that a one-time bolus of LD prodrug (e.g., LDA or LDE) be provided; and/or (ix) if the system determines that the subject consistently has a tremor at a certain time of day it may recommend a revised drug infusion regimen with an increase in the LD prodrug (e.g., LDA or LDE) infusion rate at that time of day.

The system may be programmed to recommend a one-time increase or decrease in the LD prodrug (e.g., LDA or LDE) basal infusion rate, a one-time bolus, or that a subject should skip a scheduled bolus. The system may also recommend a change to the LD prodrug (e.g., LDA or LDE) infusion regimen, such as increasing or decreasing the LD prodrug (e.g., LDA or LDE) basal infusion rate, increasing or decreasing the amount of a scheduled bolus, adding a new scheduled bolus, deleting a scheduled bolus, or changing the time of a scheduled bolus.

The system may also be programmed to similarly provide for one time increases or decreases, or to change the drug intake regimen, for other PD drugs that are being taken by the subject based on analysis of the event markers and/or input from sensors.

It will be appreciated that the pump system can be programmed to make some or all of these changes automatically, instead of simply recommending the changes to the subject.

The system may also be programmed to adjust the flow rate in order to maintain a steady LD influx in the CNS following a protein-rich meal, and thus avoid the symptoms of low brain LD, such as turning off. For example, LDEE is relatively rapidly hydrolyzed in vivo by abundant esterases. The transport to the brain is active transport, involving neutral amino acid transporters. The LD in the plasma competes with other neutral amino acids in the plasma for transport across the blood-brain barrier. The concentrations of the other neutral amino acids in plasma increase following a protein-containing meal, often reaching their peak 3-5 hours after the meal. It is therefore advantageous to gradually increase in the infused dose rate starting about 1 hour after a protein meal to reach a maximal dose rate at 3-5 hours after the meal, then decrease it, in absence of a second protein-rich meal, to base rate over about 2 hours. Thus, to maintain a steady LD influx in the CNS, the infusion rate can be adjusted to peak at about 1.7 times the base rate following consumption of a protein-rich meal.

The system may also be programmed to adjust the flow rate to accommodate the user's sleep pattern. For example, if the user prefers not to use the infusion pump while asleep, the user can start the awake period with a higher than basal infusion rate (i.e., a bolus), optionally delivered over 10-60 minutes. The system may include a diurnal program that is user specific, varied for different users to account for the times of the day when they have meals, the protein-content of the individual meals, and their sleep/awake hours.

Containers (e.g., Cartridges and Vials)

Numerous approaches are available to storing and combining the formulation components in order to achieve drug stability and convenience.

The drug product or its components (e.g., a LDEE prodrug solution, a LDEE.HCl solution, its neutralizing base, diluents, preservatives, anti-oxidants, viscosity modifiers, and/or solutions of co-infused drugs like carbidopa prodrugs) may be stored in one, two, three, four or more containers. In one preferred embodiment the storage container can also function as a drug reservoir. For example, the LD-prodrug (e.g., LDEE.HCl) pharmaceutical composition can be placed in a syringe that functions as both the container during storage and the drug reservoir when attached to a syringe pump. The distal end of the syringe can be connected to an infusion set.

In another embodiment, the storage container may include two or more sealed chambers, one chamber including a solid LD prodrug, a second chamber including a solution of HCl or another pharmaceutically acceptable acid. Optionally, the storage container may include a means for combining or mixing the two to form an infusible LD prodrug pharmaceutical composition. Examples of such a storage container are a multi-chamber syringe, and a multi-chamber drug reservoir of an infusion pump. The containers may be physically separate or they may be physically connected, e.g., separate chambers in a common housing. One or more of the containers may be configured to be connected to the infusion pump. The containers or chambers may be configured so that their contents are manually combined by the user, or so that they are automatically combined by the infusion pump. For example, a plastic barrier separating two chambers may be pierced or crushed when an actuator is pressed; the actuator may be automatically pressed when the container is inserted into the infusion pump. The contents of the containers may be combined outside the pump and then transferred to the drug reservoir. Alternatively, one of the containers or chambers may serve as the drug reservoir. The containers may be disposable or reusable. Exemplary forms of the containers are vials and syringes.

In another embodiment, the storage container includes two or more sealed chambers, each chamber including a precursor solution of an infusible LD prodrug pharmaceutical composition. One chamber includes an acidic solution comprising an LD prodrug and, optionally, a carbidopa prodrug or benserazide. A second chamber includes a solution with a more basic pH. Optionally, the storage container may include a means for combining or mixing the two or more solutions to form an infusible LD prodrug pharmaceutical composition. Examples of such a storage container are a multi-chamber syringe, and a multi-chamber drug reservoir of an infusion pump.

In yet another embodiment, the storage container includes two or more sealed chambers, the first chamber including solid LD prodrug and, optionally, benserazide or carbidopa prodrug. The second chamber includes a solution of two acids, one being preferably HCl and the second being a polybasic acid, such as phosphoric acid. Optionally, the storage container may include a means for combining or mixing the contents of the two or more chambers to form the infusible LD prodrug pharmaceutical composition. Examples of such a storage container are a multi-chamber syringe, and a multi-chamber drug reservoir of an infusion pump.

The container or chamber may contain the LD prodrug (e.g., LDA or LDE) in liquid form or in dry solid form. It may also contain benserazide or a carbidopa prodrug, e.g., its ester or amide.

When the LD-prodrug and/or carbidopa-prodrug are dissolved, the container, chamber, or drug reservoir is preferably impermeable to oxygen, e.g., constructed of glass; a non-porous ceramic; a relatively water vapor and oxygen impermeable polymer, such as polyacrylonitrile, polyvinylidene chloride, or filler loaded butyl rubber (poly(isobutylene-co-isoprene)); filler loaded chlorobutyl rubber; chlorobutyl rubber, bromobutyl rubber, butyl rubber, chlorosulfonated polyethylene (Hypalon), or amorphous polyethylene terephthalate; and metalized polymers (e.g., metalized polypropylene or polyester)). Typically the container or chamber has a wall thickness of from about 0.25 mm to about 1.5 mm (e.g., 0.25 to 0.5, 0.5 to 1.0, or 1.0 to 1.5 mm).

Materials may be selected for their compatibility with the formulation components. For example, polymers that do not increase their weight by more than 5% when soaked for 24 hours in the formulation components at 25° C. would be deemed compatible.

The container, chamber, or drug reservoir may include a vial made of glass, preferably of colored glass absorbing light of wavelengths shorter than about 450 nm. The vial may include a septum, made of a rubber, preferably inorganic filler loaded rubber, in which the permeability of oxygen is low, such as butyl rubber (poly(isobutylene-co-isoprene)); or chlorobutyl rubber or bromobutyl rubber.

The container may be hard-sided or flexible, such as a polymeric bag. The LD prodrug (e.g., LDA or LDE) can be placed into the container or chamber in such a manner that the contents of the container or chamber are substantially free of water and optionally, but not necessarily, also of oxygen. Methods of accomplishing this are well known in the art. They may include storing the composition under an inert gas. Alternatively, they may include using a vacuum to remove most gases from the container prior to or after pumping or injecting the dry solid LDE into the container, and then sealing the container.

The containers and drug reservoirs of the invention can include a connector for connection to an ambulatory infusion pump. The connector can be as simple as a septum, which is punctured to place the container in fluid communication with the pump cannula. It can also be a male or female luer lock connector to an infusion set. More complex male-female components for establishing the connection can be used to achieve the same purpose and are well known in the art.

Dry Solid Form

In one embodiment, the LD prodrug with or without the carbidopa prodrug is stored in dry solid form. The dry solid form can be the free base of the LD prodrug or the LD prodrug, i.e., the salt. The present invention includes a method of preparing for use the subcutaneously infusible pharmaceutical composition of pH 2.1-3.9 and concentration 0.15-1.6 moles per liter. Prior to use the dry solid LD prodrug (e.g., LDA or LDE) formulation is mixed with water or with an aqueous solution, or when a free base, e.g, with an HCl solution, and optionally a polybasic acid including aqueous solution, to create the infusable pharmaceutical composition. The LD prodrugs and optional DDC inhibitors (such as benserazide or carbidopa prodrugs) can be rapidly hydrolyzed in the body, and can be stored in the solid prodrug form at 25° C. for 6 months, 12 months, 18 months, or 24 months. They form infusible pharmaceutical compositions that can be stable at about 25° C. for at least 16 hours, 1 day, 2 days, 3 days, 4 days or 7 days.

The present invention includes a process for manufacturing a container or chamber containing the LD prodrug (e.g., LDA or LDE) formulation by placing the dry solid LD prodrug (e.g., LDA or LDE) formulation, in either the salt form or in the free base form, into the container. In a first embodiment, the container may include a material that is substantially oxygen and water vapor impermeable, eliminating substantially all of the water vapor and oxygen from the compartment, and the process may include sealing the container, and subsequently combining the dry LD prodrug (e.g., LDA or LDE) formulation with an aqueous solution to create a subcutaneously infusible pharmaceutical composition of pH 2.1-3.9 and concentration 0.15-1.6 moles per liter. In a second embodiment the container of the solid prodrug is stored in a second desiccated container and the process may include combining the dry LD prodrug (e.g., LDA, LDE), optionally containing benserazide or a carbidopa prodrug, with an aqueous solution to create an infusible pharmaceutical composition of pH 2.1-3.9 and concentration 0.15-1.6 moles per liter. Typically, the dry solid comprises the free base of the LD prodrug and the aqueous pharmaceutical composition includes HCl and a polybasic acid.

Optionally, the process of making a subcutaneously infusible, LD prodrug containing, pharmaceutical composition of pH 2.1-3.9 and concentration 0.15-1.6 moles per liter may also include the step of adding water or an aqueous solution to a second, optional, chamber in the container and sealing the second chamber. Optionally, the water or the aqueous solution is substantially free of dissolved oxygen and the material of the second chamber is substantially impermeable to oxygen. Optionally, the manufacturing process includes the step of the subject, or his caregiver, adding the aqueous solution to the dry solid LD prodrug (e.g., LDA or LDE) formulation, which can include the free LD prodrug base or a salt thereof. Typically in the acidic solutions the LD-prodrug is protnated, meaning that its primary amine can be an ammonium ion. The step of adding the aqueous solution may include combining the dry solid LDE with water or with an aqueous solution stored in a second chamber or container. When the free base is used, the solution stored in the second chamber or container can include an acid (e.g., HCl).

For the user of the solid prodrug, rapid dissolution of the prodrug is advantageous. Because the concentrations of the subcutaneously infused or prodrugs are generally in the range between about 0.15 M and about 1.0 M, e.g., between 0.2 M and 1.0 M, or between 0.4 M and 0.8 M, or between 0.4 M and 0.6 M the dissolution may require several minutes. To accelerate the dissolution, the prodrug particles would require a high surface-to-volume ratio, in which case the mole % of surface adsorbed-water, not removed under acceptable drying conditions, could be high. The adsorbed water could hydrolyze the LDE or LDA or carbidopa ester or carbidopa amide upon its extended storage. Resolving the conflict between fast dissolution and water content, in a particular group of embodiments of this approach, the solid stored in one container or chamber may contain the free-base LDE or LDA or carbidopa prodrug crystallites, their amines or hydrazines mostly or completely un-protonated, i.e., not protonated by an acid to form a typically more hygroscopic salt. The large basic crystallites would be, generally, advantageously less hygroscopic than the salts formed of the protonated LDE or LDA cation and the chloride, bisulfate or sulfate anion. The chamber containing the LDE or LDA (with or without the carbidopa ester or amide) may optionally also contain a buffer-forming acid and/or salt, such as citric acid, succinic acid, a sodium citrate, or a sodium phosphate in a molar amount typically less than 2 mole % or 1 mole % of the LDE or LDA. The second chamber would contain an about equivalent amount of the salt-forming acid solution, such as the hydrochloric acid solution or a slight excess of the acid, typically of about 1% of the equivalent amount or less. The stored basic LDE or LDA with or without the carbidopa ester or amide in one chamber and would be neutralized mostly by acid in the second chamber, e.g. 0.25 M-1.5 M HCl with typically 0.005 M-0.15 M of polybasic acid, e.g., about 0.3-0.8 M HCl, 0.01-0.08 M polybasic acid, or 0.4-0.8 M HCl, 0.01-0.06 M polybasic acid. Upon adding the acid to the solid base, it can dissolve in 5 minutes or less to form a subcutaneously infusible, LD prodrug containing, pharmaceutical composition of pH 2.1-3.9 and concentration 0.15-1.6 moles per liter.

The LD prodrug (e.g., LDA or LDE) with or without the benserazide or carbidopa ester or amide solid dosage form can include one or more of the following: (i) a polycarboxylic acid (with the number of carboxylic acid functions exceeding the number of amines of the free base form of the LD prodrug (i.e., free base form of LDA, LDE) and when a carbidopa prodrug is added the number of LD amines plus the number of carbidopa prodrug hydrazines. The environment of the LD prodrug molecules is thereby made acidic. In the acid environment, the catechol functions of the LD prodrug (e.g., LDA or LDE) or carbidopa prodrug molecules are less prone to oxidation, and the prodrugs are less prone to hydrolysis; (ii) a viscosity enhancing agent, which may also inhibit crystallization resulting in precipitation of large particles, in an amount such that, reconstituted the infusible formulation has a viscosity of between about 1.2 cp and about 10² cp at about 25° C.; (iii) a physiologically acceptable antioxidant (e.g., ascorbic acid, p-aminophenol or its HCl salt, acetamol, a t-butyl ortho-substituted phenol, or any antioxidant described herein); (iv) a physiologically acceptable crystal growth inhibitor (e.g., a polycarboxylic acid, collagen, albumin, polyethylene glycol, hydroxyethyl starch, dextran, glucose, glycerol, or mannitol); and (v) an enzyme inhibitor or agonist, such a DDC inhibitor like Benserazide, or the prodrug of a DDC inhibitor, like a carbidopa ester or amide, and/or a MAO-B agonist, and/or COMT inhibitor.

The solid dosage form can be packaged, for example, in a container (e.g., in a cartridge designed for insertion into an infusion pump, or a vial, the contents of which may be transferred to an infusion pump) of the invention for use in an infusion pump of the invention.

Subcutaneously Infused Compositions

In a preferred embodiment, the subcutaneously infusible pharmaceutical composition is a solution that can be both stored and infused without the step of raising the pH, such as a pharmaceutical composition having (i) an LDEE concentration between 0.15 M and 1.6 M (for example between 0.2 M and 0.3 M; 0.3 M and 0.4 M; 0.4 M and 0.5 M; 0.5 M and 0.6 M; 0.6 M and 0.7 M; 0.7 M and 0.8 M; 0.8 M and 1.0 M; or 1.0 M and 1.6 M), and (ii) a pH between about 2.0 and about 3.9 (for example, between 3.5 and 3.9 or between 3.0 and 3.5, or between 2.5 and 3.0, or between 2.4 and 2.8, or between 2.3 and 3.3, or between 2.3 and 2.9). The composition can also include a soluble DDC inhibitor like benserazide or a carbidopa prodrug.

Alternatively, the LD prodrug can be stored in liquid form, which is typically aqueous, and modified to form the infusible pharmaceutical composition. In one approach, a concentrated, acidic LD prodrug solution is stored in a first container, and a more basic solution is stored in a second container. The contents of the containers are combined to form a subcutaneously infusible, LD prodrug containing, pharmaceutical composition of pH 2.1-3.9 and concentration 0.15-1.6 moles per liter. Prior to use, enough of the solution in the second container is transferred to, or otherwise combined with, that in the first container to increase the pH, e.g., from about 2.0±0.5 to about pH 2.8±0.3. The solution in the first container or chamber can be stored without substantial LD precipitation for >3 months, >6 months, >12 months, >18 months, >24 months, >36 months, or >48 months. The stored concentrated solution is acidic, of about pH 1.5-2.0, pH 2.0-3.0 (e.g., about pH 2.8), or pH 3.0-3.9. The preferred pH of the stored solution is 2.8±0.5. The concentration of an exemplary LDEE.HCl solution is 0.15 M to 0.25 M; 0.2M to 0.3 M; 0.3 M to 0.35 M; 0.35 M to 0.45 M; 0.45 M to 0.55 M; 0.55 M to 0.65 M; 0.65 M to 0.75 M; 0.75 M to 1.0 M; 1.0 M to 2.0 M; 2.0 M to 3.0 M, 3.0 M to 3.5 M, or greater than 3.5 M. To this solution, benserazide or a carbidopa prodrug, such as carbidopa ethyl ester hydrochloride may be optionally added in a molar amount of between about 10% and about 40% of the molar amount of the LDEE.HCl. The preferred molar amount of the benserazide or carbidopa prodrug can be about 15% and 30% of the molar amount of LDEE.HCl, for example ¼ of the molar amount LDEE.HCl. The first container or chamber can be impermeable to oxygen and may include the materials previously identified in this application. A second container or chamber contains a basic solution, such as a concentrated solution of a base, optionally forming a buffer. While simple bases like sodium hydroxide or potassium hydroxide may be used, the preferred bases include a pharmaceutically acceptable potassium and/or a sodium salt of a monobasic, dibasic, tribasic or tetrabasic acid. Exemplary salts include those of citric acid; acetic acid; pyrophosphoric acid; succinic acid or phosphoric acid, like trisodium citrate, sodium acetate, tetrasodium pyrophosphate, disodium succinate or trisodium phosphate. Prior to use, enough of the solution in the second container is transferred to, or otherwise combined with, that in the first container to increase the pH e.g., from about 2.5±0.5 to about pH 4.8±0.8, or from about 2.0±0.5 to about pH 2.8±0.3.

The present invention includes a process for manufacturing a container containing the LD prodrug (e.g., LDE or LDA) formulation by placing the pharmaceutical composition of the LD prodrug (e.g., LDE or LDA) formulation into a container or chamber, the container or chamber including material that is substantially oxygen impermeable, eliminating substantially all of the water vapor and oxygen from the container or chamber, and sealing the container or chamber. Optionally, the manufacturing process includes the step combining the aqueous LDE or LDA pharmaceutical composition with a basic solution, optionally stored in a second chamber of the container. Using this method, a subcutaneously infusible, LD prodrug containing, pharmaceutical composition of pH 2.1-3.9 and concentration 0.15-1.6 moles per liter is produced.

The LD prodrug (e.g., LDA or LDE or their respective salt) aqueous liquid dosage form can include one or more of the following (i) a physiologically acceptable buffer (e.g., sodium succinate, sodium citrate, succinic acid or citric acid); (ii) a physiologically acceptable antioxidant (e.g., ascorbic acid, a salt of p-aminophenol, acetamol, a t-butyl ortho-substituted phenol, or any antioxidant described herein); (iii) a physiologically acceptable crystal growth inhibitor (e.g., a polycarboxylic acid, collagen, albumin, polyethylene glycol, hydroxyethyl starch, dextran, glucose, glycerol, or mannitol); (iv) a viscosity enhancing agent in an amount such that, reconstituted the infusible formulation has a viscosity of between about 1.2 cp and about 10² cp at about 25° C.; and (v) an enzyme inhibitor or agonist, such a DDC inhibitor, exemplified by benserazide or the carbidopa prodrugs e.g., carbidopa ester or carbidopa amide, MAO-B agonist, and/or COMT inhibitor.

The invention also features a disposable, optionally skin adhered drug container including a pharmaceutical composition of the invention. In particular embodiments the container, or a chamber of the container, includes an inert atmosphere, is substantially free of water, or substantially free of oxygen.

For subcutaneous infusions, the formulations of the invention are placed into the drug reservoir of an infusion pump device prior to use or may come pre-loaded in the drug reservoir of the device. Reservoir volumes are typically equal to or less than 3, 4, 5, 7.5, 10, 12.5, 15, 17.5, 20, 25, 30, 35 or 40 mL. The reservoir may be reusable or disposable.

The liquid dosage form can be packaged, for example, in a container of the invention for use in an infusion pump of the invention, or can be prepared just prior to infusion.

Therapy

The formulations can be subcutaneously infused to subjects in therapeutically effective amounts; for example, an amount is subcutaneously infused which prevents, delays, reduces, or eliminates the symptoms of PD. The daily subcutaneously infused molar dose of the LD-prodrug generally exceeds 25% of the total molar dose of LD and LD-prodrugs administered. For example, it is generally greater than 2.5 millimoles in a patient taking 7.5 millimoles via another route of administration (such as oral, buccal, sublingual, transcutaneous, injected, pulmonary, transcutaneous, etc.), for a total daily dosage of 10 millimoles. Typically, more than 50% of the combined total daily LD and LD-prodrug dose is subcutaneously infused, and therapy can be, for example, by infusing between 50% and 80% of the daily dose, with the rest inhaled or orally taken by the patient.

The invention features a method for treating Parkinson's disease in a subject, the method including (i) subcutaneously infusing into said subject a LD prodrug acid addition salt; and (ii) delivering LD, or a prodrug of LD, via a second route of administration other than subcutaneous infusion. The method further includes delivering 50-500 mg (e.g., 50-100, 100-200, 200-300, or 300-500 mg) of LD, or a prodrug of LD, to the subject via said second route of administration within one hour before or after initiating an infusion of the LD prodrug pharmaceutical composition. The method further includes maintaining a circulating plasma LD concentration less than 5,000 ng/mL is continuously maintained for a period of at least 8 hours during said infusion. Alternatively, doses of 50-500 mg of LD, or a prodrug of LD, are administered to the patient via said second route of administration at three or more times during the day, each dose being separated from a previous dose by at least 2 hours; and the total dose of LD, or a prodrug of LD, administered to the patient via said second route of administration during a 24 hour period is less than three times (e.g., less than two times, less than one times, less than 50%, or less than 25%) the molar dose of the infused LD prodrug acid addition salt during said 24 hour period. The second route of administration may include oral, pulmonary, or transcutaneous administration. Examples of LD delivered by the pulmonary route are disclosed in U.S. Pat. Nos. RE43,711 and 8,404,276, and in U.S. Patent Publication Nos. 20130071440, 20120111325, and 20120087952, each of which is incorporated herein by reference.

Typical infused dose ranges are from about 20 μmole/kg to about 200 μmole/kg of LD prodrug (e.g., LDA or LDE) per day. The typical daily dose of the optionally co-infused carbidopa prodrug can be between about 5 μmole/kg and about 100 μmole/kg. For example, the typical daily dose for a subject weighing 75 kg is from about 1.5 millimoles to about 15 millimoles of LD prodrug (e.g., LDA or LDE). The exemplary dosage of LD prodrug (e.g., LDA or LDE) to be administered is likely to depend on such variables as the stage of the PD patient (e.g., the dose/kg being higher for patients in more advanced stages of the disease), and the particular formulation of LD prodrug (e.g., LDA or LDE) being used. Optionally, a molar amount of benserazide or a carbidopa prodrug between about 10% and about 40% of the molar amount of the LD prodrug, for example between 15% and 30%, may be added.

In order to avoid a local rise in the decarboxylation, de-amination or trans-methylation product near the administration site that can cause local swelling, inflammation, erythema or granuloma formation or other local adverse effects an enzyme inhibitor or agonist, such a DDC inhibitor, e.g., benserazide, carbidopa, or carbidopa prodrug, a MAO-B agonist, and/or a COMT inhibitor can be co-infused in a systemically sub-therapeutic amount. The molar amount of co-infused benserazide, carbidopa, carbidopa prodrug, MAO-B agonist, and/or COMT inhibitor can be between 0.1% and 10% of the molar amount of the administered LD-prodrug. For the typically administered LD-prodrug dose range from about 20 μmole/kg to about 200 μmole/kg, the dose range of the co-infused enzyme inhibitor or agonist can be between about 20 picomole/kg and about 14 μmole/kg. For example, for local DDC inhibition the typical daily dose of the optionally co-infused carbidopa or carbidopa prodrug in a subject weighing about 75 kg can be between about 1.5 μmole and about 1 millimole.

Modes of delivery of the aqueous formulations are via fixed flow rate or programmed infusion, for formulations in which the prodrug concentration is generally between 0.15 and 1.5 M, e.g., between or 0.3 and 1 M, or 0.15 M and 0.8 M, or 0.2 M and 0.6 M, or 0.4 M and 0.6 M. The preferred regimen of delivery of the aqueous formulations is by continuous or intermittent subcutaneous infusion.

The LD prodrug concentration range in the subcutaneously infused pharmaceutical composition is generally between 0.15 M and 1.5 M. At lesser concentrations than about 0.15 M the daily subcutaneously infused volume in a patient requiring daily 5 millimoles of LD or of the prodrug may exceed 33 mL; in a patient requiring daily 10 millimoles of LD of the prodrug the daily volume may exceed 66 mL; unless distributed between multiple infusion sites, e.g., unless a multi-furcated infusion set is used and/or unless multiple pumps are used, the local infusion of such a large volume at a single site may cause edema or excessive swelling. Subcutaneous infusion of a pharmaceutical composition of a concentration greater than about 1.5 M can cause the formation of subcutaneous granulomas. The preferred concentration of the LD prodrug in the subcutaneously infused pharmaceutical composition can be between 0.15 M and 1.5 M, more preferably 0.2 M and 0.8 M, for example, 0.2±0.1 M; 0.3±0.1 M; 0.4±0.1 M, 0.5±0.1 M, 0.6±0.1 M or 0.7±0.1 M, or 0.8±0.1 M. The pH of the subcutaneously infused pharmaceutical composition is typically between 2.0 and 3.9, for example 2.4±0.3, 2.6±0.3, 2.8±0.3, 3.0±0.3, 3.2±0.3, 3.5±0.5, or 3.7±0.3. The subcutaneously administered pharmaceutical composition is typically stable, meaning clear and free of precipitated LD, for at least about 8 hrs at about 37° C., and more preferably, for at least about 16 hrs, 24 hrs, or 48 hrs.

Potential adverse effects can be ameliorated by infusing the LD prodrug (e.g., LDA or LDE) in combination with an orally taken or co-infused enzyme inhibitor or agonist, such a DDC inhibitor, e.g., benserazide, a carbidopa prodrug, MAO-B agonist, and/or COMT inhibitor; and/or anti-emetic agent, such as nicotine, lobeline sulfate, pipamazine, oxypendyl hydrochloride, ondansetron, buclizine hydrochloride, cyclizine hydrochloride, dimenhydrinate, scopolamine, metopimazine, or diphenidol hydrochloride. In certain instances it may be desirable to incorporate the anti-emetic into the formulation for simultaneous infusion in combination with the LD prodrug (e.g., LDA or LDE).

In preferred embodiments, a LD prodrug such as LDEE or LDME is subcutaneously continuously infused at least once every 60-120 minutes over a period of at least 8 hours in order to maintain a circulating plasma LD concentration greater than 400 ng/mL (e.g., greater than 400, 800, 1200, 1600 or greater than 1800 ng/mL) and less than 7,500 ng/mL (e.g., less than 5,000 ng/mL, 4,000 ng/mL, 2,500 ng/mL, or 2000 ng/mL), which is continuously maintained in the subject for a period of at least 8 hours, optionally in conjunction with an oral or injected dose of LD or LD prodrug at the start of the infusion for acceleration of the rise of the plasma LD concentration.

At the end of the infusion the circulating plasma concentration decays, the decay typically being observed in less than about 1 hour, for example in less than 45 min, or in less than 30 min. The plasma concentration does not increase by more than 50 ng/mL, 100 ng/mL, 150 ng/mL, 200 ng/mL, 250 ng/mL or 300 ng/mL at less than 1 hour after the infusion ends, for example at 45 minutes after the infusion ends or at 30 minutes after the infusion ends.

Preferred Sites and Depths of the Infusion

The preferred route of administration of the aqueous acidic formulations is subcutaneous infusion with a cannula or two or more cannulas, and/or with a needle or two or more needles, preferably administration below the dermis. Depths below the surface of the skin where the pharmaceutical compositions may be infused are between about 1 mm and about 17 mm, the preferred depth being between about 5 mm and about 10 mm.

Because the concentrations of the subcutaneously infused LD prodrug pharmaceutical compositions generally are >0.2 M, >0.3 M, 0.4 M, >0.5 M or >0.65 M it is desired that the pharmaceutical composition be rapidly diluted following its infusion. Rapid dilution reduces the likelihood and magnitude of unwanted side effects at or near the delivery site or sites. It is preferred to infuse the aqueous LD prodrug pharmaceutical composition subcutaneously at sites where the tissue-fluid is not stagnant, i.e., it flows because of abundance of arterioles and venules and/or movement of voluntary muscles or involuntary muscles; and/or proximal to major lymphatic vessels. The distance from the delivery site at which the concentration of the administered solution is halved decreases with flow, meaning it increases with the residence time, which is the inverse of the volumetric flow-rate of the tissue's fluid. Table 3, below, shows the estimated distance from the infusing orifice over which the concentration drops to ½ of the initial when the diffusion coefficient is 3×10⁻⁶ cm²s⁻¹ and the infusion rate is 3 μL min⁻¹.

TABLE 3

| | Residence time, min | | | | | | | | | | |
|---|---|---|---|---|---|---|---|---|---|---|---|
| | 1 | 2 | 3 | 4 | 5 | 6 | 7 | 8 | 9 | 10 | ∞ |
| Distance, mm | 0.45 | 0.61 | 0.73 | 0.82 | 0.9 | 0.97 | 1.04 | 1.1 | 1.15 | 1.2 | 26.5 |

For a stagnant solution the distance from the orifice to points at which the concentration drops to ½ the initial is as long as 26.5 mm. Even slight flow reduces the distance. For a residence time as long as 10 min, the distance already drops to 1.2 mm. For a 1 minutes residence time it is as short as 450 μm. During daytime and near a large and frequently used muscle or near the diaphragm, the residence time is typically less than 4 minutes and the radius of the most affected zone is less than about 820 μm. The desired flow of the treated tissue-fluid, for example the subcutaneous fluid, is effectively induced by movement of proximal large voluntary muscles that are exercised during periods in which the subject is awake. Examples of such large muscles include the trapezius, deltoid, pectoralis major, triceps brachii, biceps, gluteus maximus, sartorius, biceps femoris, rectus femoris, and gastrocnemius muscles. The desired flow of the treated subcutaneous tissue-fluid is also induced by movement of proximal large involuntary muscles exercised during periods in which the subject is either awake or asleep, such as the diaphragm. It is therefore preferred to infuse the concentrated LD prodrug pharmaceutical composition subcutaneously near these muscles. Some preferred infusion zones, for example diaphragm-moved upper/central abdominal zones, can be recognized by visible movement of the skin upon the movement of the proximal muscle, e.g., of the diaphragm upon inhalation or exhalation of air.

Multiple Point Infusion

Because concentrated and/or acidic subcutaneously infused drug pharmaceutical compositions can damage cells near the tip of the infusing cannula or needle, it is advantageous to administer through multiple orifices, i.e., cannulas and/or needles. The subcutaneous infusion can be continuous or intermittent. Their infusion orifices are spaced preferably at distances greater than about 1 cm, 2 cm, 3 cm, 5 cm, 10 cm, 15 cm, 20 cm or 30 cm. A multifurcated infusion set, such as a bi-furcated, tri-furcated, or quadri-furcated (tetra-furcated) infusion set can be used to distribute a dilute larger volume LD-prodrug solution between multiple infusion sites, such that each site is infused daily with less than about 10 mL of the drug pharmaceutical composition, for example by less than 8 mL, 6 mL, 4 mL or 3 mL. Alternatively, a skin-adhered elongated strip, of a length to width ratio of 2 or more, with two cannulas or needles typically separated by more than 1 cm, 2 cm, 3 cm, 5 cm or 10 cm can be advantageously used.

The invention features a method for subcutaneous infusion of a pharmaceutical composition, the method including: (i) providing an aqueous pharmaceutical composition including an LD prodrug (e.g., LDEE, LDME, or any LD prodrug described herein) and having a pH of from 2.1 to 4.2, (for example from 2.1 to 3.9, e.g., 2.4±0.3, 2.6±0.3, 2.8±0.3, 3.0±0.3, 3.2±0.3, 3.4±0.3 or 3.6±0.3); and (ii) subcutaneously infusing, at one or more sites, the pharmaceutical composition at a rate of less than 0.5 mL/hour (e.g., 0.5±0.1, 0.4±0.1, 0.3±0.1, 0.2±0.1, or 0.100±0.025 mL/hour) per infused site. The pharmaceutical composition can include from 0.15 to 1.6 M LD prodrug (e.g., from 0.15 M to 1.6 M, 0.15 M to 0.35 M, 0.3 M to 0.6 M, 0.5 M to 0.9 M, 0.8 M to 1.2 M, or from 1.1 M to 1.6 M LD prodrug). In some embodiments, the pharmaceutical composition is subcutaneously infused at an infusion site at a rate of less than 0.70 millimoles/hour (e.g., 0.70±0.1, 0.60±0.1, 0.50±0.1, 0.4±0.1, or 0.30±0.1 millimoles/hour). For example, the pharmaceutical composition can include 0.4±0.2 M LD prodrug (e.g., LDEE) infused at a rate of from 0.1 mL/hour to 0.35 mL/hour. The slow low pH subcutaneous infusion can be well tolerated at the infusion site and painless (post infusion).

Multiple point infusion can be carried out by a pump driving the fluid in multiple tubings, and/or cannulas, and/or needles; and/or by multiple pumps, each pump driving the fluid in one or more tubing and/or cannula and/or needle. The infusion can be through 2 or more, 4 or more, 9 or more cannulas or needles, the tips of which may be horizontally and/or vertically separated.

Optionally, two drug pumps can be used for the subcutaneous infusion, one infusing, for example in the left arm, the second in the right arm or in the abdominal region. Multiple point infusion can be also carried out with a perforated plastic cannula having one or more orifices along its length. The orifices may have similar diameters or they may differ in their diameter, for example such that the flow through the orifices will be about the same. This can be accomplished, for example, by making orifices distal from the pump larger than orifices proximal to the pump.

The prodrug containing the aqueous LD prodrug pharmaceutical composition may be delivered alternatively with a skin patch including a microneedle array in the dermis, typically at a depth of between 1 mm and about 3 mm below the epidermis. Microneedle arrays for drug delivery are described, for example, in U.S. Pat. Nos. 6,256,533, 6,379,324, 6,689,100, 6,980,555, 6,931,277, 7,115,108, 7,530,968, 7,556,821, 7,914,480, 7,785,301, 7,658,728, and 7,588,552 and in U.S. Patent Publication No. 20080269666.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

In the following examples the pH values refer, unless otherwise mentioned, to their values at the start of the experiments at about 23±2° C. The LDEE concentrations and masses reported, unless otherwise indicated, are the calculated equivalent mass of LDEE in its free base form.

### Example 1

### Histopathology Findings of Inflammation and Induration in Minipigs Receiving Continuous, Subcutaneous Infusions of Greater than 200 mg/mL LDEE, pH 4, Pharmaceutical Compositions

Two male juvenile Yucatan minipigs weighing 5-9 kg were infused subcutaneously over 16 hours with 1,142 mg (5 millimole) LDEE doses at two concentrations, 326 mg/mL (1.45 mM) and 220 mg/mL (0.98 M). The pharmaceutical compositions were citrate buffered at pH 4.6 where most, but not all, of the LDEE is in the salt form, i.e., there is also some free base. Each pig was infused on day 1 with the two simultaneous 1,142 mg doses (2,284 mg total, 5 millimoles each, 10 mmoles total) delivered by two pumps to contralateral sites and on days 10 and 24 with 1,142 mg (5 millimoles) total doses at a single site. At the 326 mg/mL (1.45 M) concentration, the flow rate was 0.216 mL/hour and the total infused volume was 3.5 mL. The dose rate per was 71 mg LDEE/hour (0.316 millimoles/hour) for all infusion sites. At the 220 mg/mL (0.98 M) concentration, the flow rate was 0.328 mL/hour and the total infused volume was 5.2 mL.

Most, but not all, of the infused sites were swollen and/or developed firm indurations. These swollen or firm sites were biopsied one week, three weeks and one month after the infusions. Microscopic changes associated with the infusion were observed in samples taken from the swollen and or palpably hard infused sites one week, three weeks, and one month after the infusions. One week after infusion of the 220 mg LDEE per mL (0.98 M LDEE), pH 4.6 pharmaceutical composition, mild thrombus formation in the blood vessels of the panniculus, with minimal hemorrhage and subacute inflammation associated were observed. Minimal subacute inflammation in the panniculus was the only finding. A minimal to mild amount of inspissated brown matter was present in the apocrine glands of both animals. Three weeks after infusion of the 220 mg LDEE per mL (0.98 M LDEE), pH 4.6 pharmaceutical composition, minimal hemorrhage of the dermis and panniculus were observed in minipig A; but in minipig B mild granulomatous inflammation characterized by infiltration of histiocytes, lymphocytes, and formation of multinucleated giant cells, with moderate associated fibrosis were observed. An intracytoplasmic brown material was also observed in the areas of inflammation. One month after infusion of the 328 mg LDEE/mL (1.45 M LDEE), pH 4.6 pharmaceutical composition, mild to moderate granulomatous inflammation of the panniculus was observed. The granulomatous inflammation was characterized by infiltration of histocytes and lymphocytes in addition to variable amounts of multinucleated giant cells. The inflammatory process was seen in the panniculus and involved the adipose tissue. Variable degree of fibrosis, hemorrhage and intracytoplasmic brown material were associated with the inflammatory process. Occasionally, degeneration and/or necrosis of the myofiber (when present) occurred in the panniculus carnosus.

### Example 2

### Infusion Site Reactions Caused by Intermittent Subcutaneous Infusions of a 200 mg/mL LDEE (0.89 M LDEE) Pharmaceutical Composition of pH 4.5 in Minipigs

Over the course of two consecutive days, four 6-12 week old juvenile minipigs weighing about 6-13 kg were each administered six subcutaneous infusions of 140 mg (0.62 millimoles) of LDEE. Each 140 mg infusion was administered over a period of 8 hours in a pH 4.5 (pH adjusted with trisodium citrate) pharmaceutical composition (mostly LDEE.HCl, but also containing some free base), and in a volume of 0.705 mL, i.e., the LDEE concentration was 200 mg/mL (calculated mass of LDEE in its free base form), i.e. 0.89 M. At each infused site, the minipig received 8 boluses of 0.088 mL with a delivery period of 5 minutes per bolus and a non-delivery period of 55 minutes.

Each infusion site was spaced a minimum of 2.5 inches (6.3 cm) apart from any other infusion site. All minipigs received seven doses of oral carbidopa, 25 mg/dose, at: 12 hours prior to the infusion; 3 doses on the first day of infusion; 3 doses on the second day of infusion.

The rate of infusion site reactions is reported in Table 4 below. In all cases, the infusion site reaction constituted a swelling of 0.5-4.0 cm diameter, typically 0.5-1.5 cm. The data demonstrate that a dose of 140 mg of LDEE in a 200 mg/mL (0.89M), pH 4.5 pharmaceutical composition can safely be subcutaneously infused, albeit with mild infusion site reactions and a 33% rate of small granuloma formation at 14 days post-infusion.

TABLE 4
# of Infusion Site Reactions/# of Infusion Sites

| Immediately | 3 Days | 10 Days | 15 Days |
|---|---|---|---|
| Post-Infusion | Post-Infusion | Post-Infusion | Post-Infusion |
| 19/24 | 10/24 | 15/24 | 8/24 |

### Example 3

### Reduced Infusion Site Reactions with Intermittent and Continuous Subcutaneous Infusions of a 100 mg/mL LDEE Pharmaceutical Composition of pH 4.5 in Minipigs

Over the course of two consecutive days, four 6-12 week old juvenile minipigs weighing about 6-13 kg were each administered six sets of subcutaneous infusions of 281 mg (1.25 millimoles) of LDEE. Each 281 mg infusion was administered over a period of 16 hours in a pH 4.5 (pH adjusted with trisodium citrate) pharmaceutical composition (mostly LDEE.HCl, but also containing some free base) and in a volume of 2.81 mL, i.e., the LDEE concentration was 100 mg/mL (calculated mass of LDEE in its freebase form), i.e. 0.44 M LDEE.

Each infusion site was spaced a minimum of 2 inches (5 cm) apart from any other infusion site. All minipigs received seven doses of oral carbidopa, 25 mg/dose, at: 12 hours prior to the infusion; 3 doses on the first day of infusion; 3 doses on the second day of infusion.

There were four treatment groups:

A: Six infusions, intermittent with ibuprofen. In each infusion, the minipig received 16 boluses of 0.176 mL with a delivery period of 5 minutes per bolus and a non-delivery period of 55 minutes. The minipig was also administered 200 mg oral doses of ibuprofen at 12 hours prior to the infusion, then twice daily on days 1-15 post-infusion, i.e., daily doses of 400 mg.

B: Six infusions, intermittent with no ibuprofen. In each infusion, the minipig received 16 boluses of 0.176 mL with a delivery period of 5 minutes per bolus and a non-delivery period of 55 minutes.

C: Six infusions, continuous infusion with ibuprofen. In each infusion, the minipig was infused with the LDEE pharmaceutical composition continuously at a rate of 0.176 mL/hour. The minipig was also administered 200 mg oral doses of ibuprofen at 12 hours prior to the infusion, then twice daily on days 1-15 post-infusion, i.e., daily doses of 400 mg.

D: Six infusions, continuous infusion with no ibuprofen. In each infusion, the minipig was infused with the LDEE pharmaceutical composition continuously at a rate of 0.176 mL/hour.

The rate of infusion site reactions is reported in Table 5 below. In all cases, the infusion site reaction constituted a firm swelling of 0.5-1.5 cm diameter. The data demonstrate that a dose of 281 mg (1.25 millimole) of LDEE in a 100 mg/mL (0.44 M), pH 4.5 pharmaceutical composition can be safely subcutaneously infused with minimal infusion site reactions and no granuloma formation, in 24 out of 24 infusions. The data suggest that intermittent dosing has a lower rate of infusion site reactions than continuous dosing. The data also suggest that oral Ibuprofen therapy does not reduce the rate of infusion site reaction. Compared to the infusions in Example 1, the data demonstrate that subcutaneous infusion of LDEE pharmaceutical compositions of 100 mg/mL (0.44 M LDEE concentration) split among multiple sites are significantly better tolerated than infusions of pharmaceutical compositions of 200 mg/mL (0.89 M LDEE concentration) administered at a single site (infusion site reactions in 0 out of 24 infusions versus 8 out of 24 infusions at 14-15 days post-infusion).

TABLE 5
# of Infusion Site Reactions/# of Infusion Sites

| | Immediately | 3 Days | 10 Days | 15 Days |
|---|---|---|---|---|
| | Post- | Post- | Post- | Post- |
| | Infusion | Infusion | Infusion | Infusion |
| Treatment A - | 0/6 | 2/6 | 5/6 | 0/6 |
| Intermittent | | | | |
| with Ibuprofen | | | | |
| Treatment B - | 0/6 | 1/6 | 1/6 | 0/6 |
| Intermittent, | | | | |
| no Ibuprofen | | | | |
| Treatment C - | 0/6 | 2/6 | 6/6 | 0/6 |
| Continuous | | | | |
| with Ibuprofen | | | | |
| Treatment D - | 0/6 | 4/6 | 1/6 | 0/6 |
| Continuous, | | | | |
| no Ibuprofen | | | | |

### Example 4

### Reduced Infusion Site Reactions with Intermittent, Subcutaneous Infusion of 100 mg/mL and 200 mg/mL (0.89 M) LDEE Pharmaceutical Compositions of pH 3.7 in Minipigs

Over the course of two days, four 6-12 week old juvenile minipigs weighing about 6-13 kg were each administered eight subcutaneous infusions of LDEE.HCl pharmaceutical compositions at pH 3.7 (pH adjusted with trisodium citrate), where the fraction of free base in the solution was greatly reduced relative to that at pH 4.5 or 4.6 in Examples 1-3. Concentrations of 100 mg/mL (0.44 M) and 200 mg/mL (0.89 M) and doses of 140, 282 and 563 mg (calculated mass of LDEE in its freebase form), i.e., respective doses of 0.62, 1.25 and 2.5 millimoles, were administered over 8, 16 and 16 hours respectively. All infusions were intermittent, with a delivery period of 5 minutes per bolus and a non-delivery period of 55 minutes, except for those where a dose of 563 mg, i.e. 2.5 millimoles, was infused at a concentration of 100 mg/mL (0.44 M). In these the delivery period was 5 minutes per bolus and the non-delivery period was 35 minutes.

Each infusion site was spaced a minimum of 2 inches (5 cm) apart from any other infusion site. All minipigs received eight doses of oral carbidopa, 25 mg/dose: one dose at 12 hours prior to the infusion on the first day; 3 doses on the first day of infusion; one dose at 12 hours prior to the infusion on the second day; and 3 doses on the second day of infusion.

The rate of infusion site reactions is reported in Table 6 below. The data demonstrate that doses of 140, 282 and 563 mg (0.62, 1.25 and 2.5 millimoles) of LDEE in 100 mg/mL (0.44M) and 200 mg/mL (0.89 M) pharmaceutical compositions of pH 3.7 can be safely subcutaneously infused with minimal infusion site reactions, and that there are no infusion site reactions on the 14^{th} post-infusion day. The data further suggest that subcutaneous infusion of 140, 282 and 563 mg (i.e. 0.62, 1.25 and 2.5 millimole) doses of LDEE in 100 mg/mL (0.44 M) pharmaceutical compositions are better tolerated than in 200 mg/mL (0.89 M) pharmaceutical compositions (infusion site reactions in 0 out of 16 infusions for the 100 mg/mL concentration versus 5 out of 16 infusions for the 200 mg/mL concentration at 14 days post-infusion). The data further show that infusion of LDEE pharmaceutical composition volumes between 0.094 and 0.37 mL per hour per site is well tolerated.

TABLE 6
# of Infusion Site Reactions/# of Infusion Sites

| Conc. | Dose | Duration | # of | | | | | | |
|---|---|---|---|---|---|---|---|---|---|
| (mg/mL) | (mg) | (hrs) | Infusions | 0 hrs | 12 hrs | 24 hrs | 3 days | 7 days | 14 days |
| 100 | 140 | 8^{a} | 8 | 0/8 | 0/8 | 0/8 | 3/8 | 1/8 | 0/8 |
| 100 | 282 | 16^{b} | 4 | 0/4 | 0/4 | 1/4 | 1/4 | 0/4 | 0/4 |
| 100 | 563 | 16^{c} | 4 | 0/4 | 0/4 | 2/4 | 3/4 | 3/4 | 0/4 |
| 200 | 140 | 8^{a} | 8 | 0/8 | 0/8 | 0/8 | 3/8 | 3/8 | 2/8 |
| 200 | 282 | 16^{b} | 4 | 0/4 | 0/4 | 0/4 | 2/4 | 2/4 | 1/4 |
| 200 | 563 | 16^{d} | 4 | 0/4 | 0/4 | 0/4 | 3/4 | 3/4 | 2/4 |

^{a}The time between the first and the 8th bolus was 7 hours. The boli were of 0.176 mL for the 100 mg/mL pharmaceutical composition and of 0.088 mL for the 200 mg/mL pharmaceutical composition.
^{b}The time between the first and the 16th pulse (bolus) was 15 hours. The boli were of 0.176 mL for the 100 mg/mL pharmaceutical composition and of 0.088 mL for the 200 mg/mL pharmaceutical composition. The average hourly flow rate for the 100 mg/mL pharmaceutical composition in the 15 hour period was 0.094 mL.
^{c}The time between the first and the 23rd pulse (bolus) was 15 hours and 20 min. The boli were of 0.244 mL. The average hourly flow rate in the 15 hour 20 minutes period was 0.37 mL.
^{d}The time between the first and the 16th pulse (bolus) was 15 hours. The boli were of 0.176 mL. The average hourly flow rate in the 15 hour period was 0.19 mL.

### Example 5

### Greatly Reduced Infusion Site Reactions with Continuous Subcutaneous Infusion of a 100 mg/mL (0.44 M) LDEE Pharmaceutical Composition of pH 3.7 in Minipigs

During two days of infusion separated by one week, four juvenile minipigs weighing between 6.0 kg and 8.3 kg were each continuously infused for 8 hours with 5.6 mL of a 100 mg/mL (0.44 M LDEE), pH 3.7 citrate buffered LDEE pharmaceutical composition over 8 hours. At the pH of 3.7 the fraction of free base in the solution was greatly reduced relative to its fraction at pH 4.5 or 4.6 in Examples 1-3. The 5.6 mL volume was divided between two simultaneously infused sites, each site receiving 2.8 mL, i.e., 280 mg LDEE. At each site the flow rate was 0.35 mL/hour, i.e. 35 mg LDEE/hour was infused. The number of sites infused on the first day totaled in the four minipigs 8, and was 7 on the 8^{th} day, because of dislodgement of a cannula from one site. The total number of infused sites was 15. Table 7 shows the ratio of sites with observable reactions and the total number of infused sites. The reactions had an about 1 cm diameter, were slightly raised and firm.

TABLE 7
Fraction of Infused Sites with Firmness
for pH 3.7, 100 mg/mL (0.44M) LDEE
N = 15 Infusions

| | Time after end of infusion | | | | | |
|---|---|---|---|---|---|---|
| | 0 hrs | 12 hrs | 24 hrs | 3 days | 7 days | 14 days |
| Fraction of sites | 0.067 | 0.067 | 0.067 | 0.067 | 0.067 | 0 |
| with a reaction | | | | | | |

The experiment showed that the fraction of sites with reactions is reduced to 1/15 when the dose is divided between two continuously infused sites, the pH is reduced and the infused LDEE pharmaceutical composition concentration is 100 mg/mL (0.44 M LDEE) and that the reactions are transient, with no reaction seen after two weeks.

### Example 6

### Greatly Reduced Infusion Site Reactions with Subcutaneous Infusion of 50 mg/mL (0.22 M) LDEE Pharmaceutical Composition of pH 3.7 in Minipigs

Over the course of two consecutive days, four 6-12 week old juvenile minipigs weighing about 6-13 kg were each administered 18 subcutaneous infusions of LDEE pharmaceutical compositions at pH 3.7 (LDEE.HCl, pH adjusted with trisodium citrate). At the pH of 3.7 the fraction of free base in the solution was greatly reduced relative to its fraction at pH 4.5 or 4.6 in Examples 1-3. Intermittent and continuously infused doses of 137.5-140 mg (0.61-0.62 millimoles) were administered over 8 hours, and intermittent doses of 275 mg (1.22 millimoles) were administered over 16 hours (calculated mass of LDEE in its freebase form). All intermittent infusions had a delivery period of 5 minutes per bolus and a non-delivery period of 40 minutes.

Each infusion site was spaced a minimum of 2 inches (5 cm) apart from any other infusion site. All minipigs received seven doses of oral carbidopa, 25 mg/dose: one dose at 12 hours prior to the infusion on the first day; 3 doses on the first day of infusion; and 3 doses on the second day of infusion.

The rate of infusion site reactions is reported in Table 8 below. The data demonstrate that doses of 137.5-140 mg and 275 mg of LDEE in 50 mg/mL pharmaceutical compositions of pH 3.7 can be safely subcutaneously infused, either continuously or intermittently. The data further show that infusion of LDEE pharmaceutical composition volumes of about 0.34 mL per hour per site is well tolerated.

TABLE 8
# of Infusion Site Reactions/# of Infusion Sites

| | | Dura- | | # of | | | | |
|---|---|---|---|---|---|---|---|---|
| Conc. | Dose | tion | Delivery | Infu- | 0 | 12 | 24 | 3 |
| (mg/mL) | (mg) | (hrs) | Type | sions | hrs | hrs | hrs | days |
| 50 | 137.5 | 8 | Intermittent | 8 | 0/8 | 0/8 | 0/8 | 3/8 |
| 50 | 140 | 8 | Continuous | 4 | 0/4 | 0/4 | 0/4 | 0/4 |
| 50 | 275 | 16 | Intermittent | 6 | 0/6 | 0/6 | 1/6 | 1/6 |

### Example 7

### Skin Symptoms May Correlate with Result of LD or LDEE Accumulation at the Infused Site, Possibly Causing a Post-Infusion Increase in the Plasma LD-Concentration

Six minipigs, labeled A, B, C, D, E and F, weighing between 5 and 8 kg, were ported for venous access two weeks prior to infusion. The minipigs received three 25 mg oral Lodosyn doses, one 12 hours before the start of the infusion, the second at the start of the infusion, the third 4 hours after the start of the infusion.

On the first day minipigs A and B were infused over 8 hours continuously with 90 mg (400 micromoles) of a 100 mg/mL (0.44 M), i.e., with 0.9 mL of the pH 3.7 (16 mM citrate buffered) LDEE pharmaceutical composition at a flow rate of 0.113 mL/hour and at a dose rate of 50 micromoles per hour. On Day 8 minipig A was similarly re-infused at the contralateral body site after it was pre-infused over about 10 minutes with 15 units of human hyaluronidase.

Minipigs C and D were infused on the first day over 8 hours continuously with 560 mg (2.5 millimoles) of the 100 mg/mL, i.e., with 5.6 mL of the pH 3.7 (16 mM citrate buffered) LDEE pharmaceutical composition. The 560 mg dose was split between 2 sites separated by 3 inches, each site receiving 280 mg. Two pumps were used, each pump delivering 2.8 mL over 8 hours, i.e., the flow rate at each infused site was 0.35 mL/hour and the LDEE dose rate at each site was 35 mg/hour, the combined dose rate per minipig being 70 mg LDEE/hour, equaling 311 micromoles LDEE/hour.

Minipigs E and F were infused on Day 1 similarly to minipigs C and D, but were pre-infused over about 10 minutes with 15 units of human hyaluronidase.

Because the juvenile minipigs were growing rapidly, i.e., their weight increased between Day 1 and Day 8, while the infused doses were the same, the plasma LD concentrations were lower on Day 8 than they were on Day 1.

On the 8th day minipig D was re-infused at the Day 1 infusion sites over 8 hours continuously with 560 mg (2.5 millimoles) of 100 mg/mL, i.e., with 5.6 mL of the pH 3.7 (16 mM citrate buffered) LDEE pharmaceutical composition. The 560 mg (2.5 millimole) dose was again split between the two sites infused on Day 1, that were separated by 3 inches, each site receiving 280 mg (1.25 millimoles). Two pumps were used, each pump delivering 2.8 mL over 8 hours, i.e., the flow rate at each infused site was 0.35 mL/hour and the LDEE dose rate at each site was 35 mg/hour (156 micromoles/hour), the combined dose rate being 70 mg LDEE/hour, equaling 311 micromoles LDEE/hour. Minipig F was also re-infused on Day 8 similarly to minipig D but was pre-infused with 15 units of human hyaluronidase over about 10 min.

On Day 8, minipig C was also re-infused, but received only half the dose. Only one of two the day 1 sites was re-infused over 8 hours, continuously, with 280 mg (1.25 millimoles) of a 100 mg/mL (0.44 M LDEE), i.e., with 2.8 mL of the pH 3.7 (16 mM citrate buffered) LDEE pharmaceutical composition. Only one pump was used, delivering 2.8 mL over 8 hours, i.e., the flow rate at the infused site was 0.35 mL/hour and the LDEE dose rate at the site and in the animal was 35 mg/hour, equaling 155 micromoles LDEE/hour.

Blood samples collected included samples at 8 hours (end of infusion), 40 minutes after the end of the infusion, 100 minutes after the end of the infusion, and 170 minutes after the end of the infusion. The samples were rapidly cooled and spun down to plasma; because hemoglobin reacts with LD in a reaction where LD is decarboxylated, red-colored plasma samples were excluded.

The minipigs were inspected for skin symptoms just after the end of the infusions then at 12 hours, 24 hours, 3 days, 7 days and 14 days after the end of the infusions. Of the six minipigs providing blood samples that were not red, only minipigs D had a reaction, an about 1 cm diameter slightly raised swelling. The swelling was observed at one of the animal's infused sites 7 days after the first infusion, then again at the same site after the end of the 8th day reinfusion.

The plasma LD concentrations at the end of the infusions and at 40 min, 100 minutes and 170 minutes after the end of the infusions are shown in Table 9.

TABLE 9
Plasma LD concentrations at the end
and after the end of the infusions

| | End of | 40 minutes | 100 minutes | 170 minutes |
|---|---|---|---|---|
| | Infusion | Post-Infusion | Post-Infusion | Post-Infusion |
| | Plasma LD, | plasma LD, | plasma LD, | plasma LD, |
| Minipig/day | ng/mL | ng/mL | ng/mL | ng/mL |
| A/1 | 1,051 | 1,093 | | |
| B/1 | 1,351 | 1,010 | 686 | 376 |
| C/1 | 11,377 | 11,006 | 6,010 | 5,754 |
| D/1 | 15,356 | 17,055 | 9,790 | 6,942 |
| E/1 | 14,212 | 11,918 | 5,464 | 7,329 |
| F/1 | 12,982 | 12,310 | 3,046 | 5,903 |
| A/8 | 679 | 691 | 342 | 185 |
| C/8 | 4739 | 4551 | 3357 | 2306 |
| D/8 | 8075 | 8350 | 5928 | 4219 |
| F/8 | 8880 | 8223 | 6231 | 3650 |

As seen in Table 9, in most animals the plasma concentration declined at 40 minutes after cessation of the infusion, but in the skin-symptom showing minipig D it increased 40 minutes after the cessation of the Day 1 infusion by 1,699 ng/mL and increased again by 275 ng/L 40 minutes after cessation of the day 8. The animal showed both delayed and prompt swellings of about 1 cm diameter at the infused site.

The infusion of LDEE at a dose rate high enough to raise the plasma LD concentrations above 10,000 ng/mL, even above 15,000 ng/mL, respectively more than threefold and fivefold higher than the plasma concentrations of LD in advanced PD patients receiving about 2 g of LD daily, did not result in a readily observable change in the behavior of the minipigs, suggesting that the very high dose rates per kg were well tolerated.

The experiment suggests that skin symptoms may result from LD or LDEE accumulation at the infused site, which is also the cause of post-infusion increase in the plasma LD-concentration. The experiment also suggests that in order to avoid reactions like post-infusion swelling and/or firmness and/or the symptoms revealed by the biopsies of Experiment 1, it is advantageous to infuse compositions that do not cause subcutaneous depot formation. For example, it is advantageous to infuse solutions using methods such that, at or near 40 minutes post-infusion, there is a decrease or only a small increase in the plasma LD concentration.

### Example 8

### Rapid Hydrolysis of LDEE to LD Upon its Subcutaneous Infusion

In addition to measuring the LD plasma concentrations as described in Example 6, also the plasma LDEE concentrations were measured. The plasma LDEE concentrations, as shown in Table 10, averaged about 1/1000th of the concentrations of LD, showing that the subcutaneously infused LDEE was rapidly hydrolyzed after its infusion to LD (and ethanol).

TABLE 10
Plasma concentrations of LDEE and LD after 8 hours of infusion

| | Plasma LDEE, | |
|---|---|---|
| | ng/mL, 480 minutes | Plasma LD, ng/mL, |
| | after start of | 480 minutes after |
| Minipig/day | infusion | start of infusion |
| A/1 | 3 | 1,051 |
| B/1 | 3 | 1,351 |
| C/1 | 19 | 11,377 |
| D/1 | 13 | 15,356 |
| E/1 | 8 | 14,212 |
| F/1 | 9 | 12,982 |
| A/8 | 0 | 679 |
| C/8 | 2 | 4739 |
| D/8 | 6 | 8075 |
| F/8 | 6 | 8880 |
| Average | 6.9 | 7,870 |

### Example 9

### Long Refrigerated Shelf Life of an Initially 0.44 M LDEE.HCl, pH 3.7±0.1 Solution

The LD concentration was monitored in 3 samples of 0.44 M LDEE.HCl at pH 3.7±0.1 and 4.0° C. After 39 weeks of refrigerated storage the concentration of LD was observed to be less than 2 mg/mL, well below the solubility limit of LD.

### Example 10

### Painless and Symptom-Less Continuous Subcutaneous Infusion of Acidic Citric Acid Solutions of pH 2.4 and pH 2.6 in a Human Volunteer

Because it was observed that at 100 mg/mL LDEE concentration, where the LDEE.HCl concentration is 0.44 M, there are fewer and lesser skin indurations/swellings at pH 3.7 than at higher pH, e.g., near pH 4.6, an experiment was conducted to test infusion of even lower pH solutions. It is hypothesized that such lower pH infusions could be beneficial because a lower pH at the infused site could: (a) decrease the rate of hydrolysis of LDEE to less soluble LD and ethanol; (b) decrease the rates of local O₂-oxidation of LDEE and LD (their oxidation rates being slower at lower pH); (c) decrease the deposition of free or bound LD; and (d) increase dilution before the LDEE salt is neutralized, i.e., the free base is formed. Solutions were subcutaneously infused in a 79 year old male human volunteer to test for tolerability of infusion of solutions having a pH between pH 2 and pH 3. Based on the literature and discussions with physicians, the infusion of such strongly acidic solutions was anticipated to be painful, tissue damaging and therefore clinically unacceptable. In the experiments, between 2.5 mL and 2.8 mL of each of 3 solutions was subcutaneously infused at 0.35 mL/hr flow rate and at 9 mm depth over between 7 and 8 hours. All infusions were with the Medtronic Paradigm Pump and Medtronic MMT-975 Mio 9 mm (cannula length, vertically inserted) 80 cm (tubing length) infusion sets. The subcutaneously, continuously infused sterile solutions were (1) 0.1 M citric acid, with a pH of 2.1; (2) 33 mM citric acid, with a pH of 2.4; and (3) 33 mM citric acid in 0.44 M NaCl, with a pH of 2.6. Solution #3 was thought to approach in its osmolyte concentration that of 0.44 M LDEE.HCl, which is the 100 mg/mL LDEE pharmaceutical composition infused in minipigs. Solution 1 was infused in the abdomen, about 10 cm below and 7.5 cm to left of the sternum; Solution 2 was infused in the front side of the left upper arm, 10 cm below shoulder and 13 cm above elbow; Solution 3 was infused in the outer side of the left upper arm, 8 cm below shoulder and 14 cm above elbow.

Infusion of Solution 1, the pH 2.0, 0.1 M citric acid solution, caused only slight discomfort during infusion and very slight swelling/induration at 12 hours post-infusion, which resolved after 24 hours. Infusion of Solution 2, the pH 2.4, 33 mM citric acid solution, was not felt, caused no discomfort, and did not result in any visible or palpable change in the infused site. Similarly, infusion of Solution 3, the pH 2.6, 33 mM citric acid, 0.44 M NaCl solution, was painless, unfelt and did not result in any symptom, i.e., it did not result in any visible or palpable change in the infused site.

The experiment showed that citric acid solutions, having a pH of pH 2.4 or 2.6, without or with a 0.44 M osmolyte (NaCl), can be painlessly infused at 0.35 mM/h flow and that their infusion causes no visible or palpable change at the infused site. The experiment suggests that storable, >1 year shelf-life, acidic, 100 mg/mL (0.44 M) LD prodrug pharmaceutical compositions (such as LDEE.HCl pharmaceutical compositions), of a pH as low as about 2.4±0.3 and/or 2.7±0.3 could also be painlessly infused and that their acidity may not cause swelling or firmness or inflammation at the infused site. Infusion of the more acidic pharmaceutical composition is expected to further reduce the likelihood, or even eliminate, the already infrequent swelling and induration associated with infusion of about 100 mg/mL (about 0.44 mM) LDEE. It can be reasonably expected that moderately higher infusion rates (e.g., 35-70 mM/h, or greater than 70 mM/h) and larger doses (e.g., 6 or 10 mL) may also be well tolerated.

### Example 11

### Estimation of the Lower pH Threshold for Painless and Symptom-Less Continuous Subcutaneous Infusion of Acidic Citric in a Human Volunteer

Using a Medtronic Minimed Paradigm 723 insulin pump with a Medtronic Quickset Paradigm 6 mm canula (32 inch long tubing) infusion set, a 79 year old volunteer infused subcutaneously in his abdominal fat 2.91 mL of a sterile 0.1M citric acid solution; the pH of the solution was about 2.1. The continuous infusion was at a flow rate of 0.35 mL/hour; the vertically inserted cannula was 6 mm long, i.e., the solution was infused 6 mm below the epidermis. Although the infusion did not cause pain, it was slightly irritating, giving rise to a sensation of local tightness and pinching. At the end of the infusion there were no symptoms, i.e., there was no redness or swelling, nor did any post-infusion symptom appear in the month following the infusion.

The experiment confirmed the result of Experiment 9 (Solution 1), i.e., that although the infusion of the pH 2.0 solution in the abdominal fat is felt, the pain is minimal.

### Example 12

### Absence of Pain or Irritation Upon Continuous Infusion of a 10 mM, pH 3.0 Citric Acid, 0.9 Weight/Volume % NaCl Saline Solution at 0.35 mL/Minutes Flow Rate

Using a Medtronic Minimed Paradigm 723 insulin pump with a Medtronic Quickset Paradigm 6 mm canula (32 inch long tubing) infusion set, a 79 year old volunteer infused subcutaneously in his abdominal fat 2.82 mL of a sterile 10 mM citric acid, 0.9 weight/volume % NaCl solution over about 8 hours; the pH of the solution was about 3.0. The continuous infusion was at a flow rate of 0.35 mL/hour; the vertically inserted cannula was 6 mm long, i.e., the solution was infused 6 mm below the epidermis. The infusion caused no pain or irritation. At the end of the infusion there were no symptoms, i.e., there was no redness or swelling, nor did any post-infusion symptom appear in the month following the infusion.

The experiment shows that a pH 3.0 saline solution can be painlessly infused and that its infusion does not cause a visible or palpable change at or near the infused site.

### Example 13

### Regimens with One Hour Long Non-Delivery Periods Following a Two Hour Long Infusion Delivery Period Reduces Skin Symptoms

The same 79 year old healthy volunteer was infused with the same volumes of the same acidic solution on three different days at three different sites of the skin with two LDEE.HCl doses, each dose of 1.25 millimoles. In both infusions cannulas of 9 mm length were vertically inserted and their tips resided in subcutaneous tissue. The infused solution was 0.48 M LDEE.HCl, buffered with sodium citrate and citric acid to pH 3.5.

The first infusion was continuous over 16 hours, such that the dose rate was 17.6 mg/hour. In the continuous infusion the flow rate was 0.163 mL/hour and the volunteer did not take orally carbidopa. The second infusion was intermittent over 10.5 hours according to the following schedule:
1. 2 hour infusion
2. 1 hour non-infusion
3. 2 hour infusion
4. 1 hour non-infusion
5. 2 hour infusion
6. 1 hour non-infusion
7. 1.5 hour infusion

The dose rate was 37.8 mg/hour in each of the four infusion periods. The flow rate was 0.35 mL/hour. During the second infusion, the volunteer took two 25 mg pills of carbidopa prior to the infusion and two 25 mg pills of carbidopa during the infusion.

The third infusion was intermittent over 10.8 hours according to the following schedule:
1. 2 hour infusion
2. 1.3 hours non-infusion
3. 2 hour infusion
4. 1 hour non-infusion
5. 2 hour infusion
6. 1 hour non-infusion
7. 1.5 hour infusion

The dose rate was 37.8 mg/hour in each of the four infusion periods. The flow rate was 0.35 mL/hour. During the third infusion, the volunteer did not take pills of carbidopa.

There was no pain at any time during the infusions. At the end of the first continuous infusion there was a 2.5 cm diameter slightly protruding hard swelling and there were two 2 mm diameter hematomas about 1.5 cm from the infusion site; the skin was redder than the surrounding skin over a 3 cm diameter area. The swelling and redness persisted for 12 hours and subsided after 21 hours when a 1 cm diameter palpable induration was left. After 2 days the induration had a 0.5 cm diameter and the two hematomas 1.5 cm from the infused site were still visible.

In the intermittent second infusion there were no skin symptoms at the end of the infusion. After 11 hours there appeared a barely perceptible very lightly pink 2.5 cm diameter zone and a very mild soft palpable swelling; after 36 hours there remained only a barely palpable 3 cm long 1 cm wide soft horizontal induration.

In the intermittent third infusion, without oral carbidopa, there were no significant skin symptoms at the end of the infusion. The infusion showed that the cause of alleviation or avoidance of symptoms at the end of the infusion was the intermittent infusion, not the oral carbidopa. Four hours after the end of the infusion the skin was very slightly more pink over a 2.5 cm diameter zone and there was a small (2 cm×1 cm) barely palpable induration. Fourteen hours after the end of the infusion the site could be recognized by its pink color. There is a very slightly raised area.

The experiment shows that acute post-infusion inflammation can be alleviated or avoided by one hour pauses between infusions at a site. With multiple sites all the infusions can be turned on an off simultaneously, or alternatively they can be rotated. For example, there could be at any instant sites that are infused and sites at which the infusion is suspended, e.g., for a period between 10 min and 2 hours, for example between 30 min and 1 hour, or between 1 hour and 2 hours.

### OTHER EMBODIMENTS

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A pharmaceutical composition comprising an aqueous solution containing from 0.15 to 1.6 M LD prodrug acid addition salt and having a pH of from 2.1 to 3.9, wherein said pharmaceutical composition is subcutaneously infusible.

2. The pharmaceutical composition of claim 1, wherein said LD prodrug acid addition salt is an acid addition salt of LDEE or LDME.

3. The pharmaceutical composition of claim 1 or 2, wherein said pharmaceutical composition comprises an aqueous solution containing from 0.15 to 0.7 M LD prodrug acid addition salt.

4. The pharmaceutical composition of claim 1 or 2, wherein said pharmaceutical composition comprises an aqueous solution containing from 0.7 to 1.6 M LD prodrug acid addition salt.

5. The pharmaceutical composition of any one of claims 1-4, wherein said pharmaceutical composition has a pH of from 2.1 to 3.0.

6. The pharmaceutical composition of claim 5, wherein said pharmaceutical composition has a pH of 2.4±0.3.

7. The pharmaceutical composition of claim 5, wherein said pharmaceutical composition has a pH of 2.6±0.3.

8. The pharmaceutical composition of any one of claims 1-4, wherein said pharmaceutical composition has a pH of from 3.1 to 3.9.

9. The pharmaceutical composition of any one of claims 1-4, wherein said pharmaceutical composition has a pH of 2.8±0.3.

10. The pharmaceutical composition of any one of claims 1-4, wherein said pharmaceutical composition has a pH of 3.1±0.3.

11. The pharmaceutical composition of any one of claims 1-4, wherein said pharmaceutical composition has a pH of 3.4±0.3.

12. The pharmaceutical composition of any one of claims 1-4, wherein said pharmaceutical composition has a pH of 3.7±0.2.

13. The pharmaceutical composition of any one of claims 1-12, wherein said pharmaceutical composition further comprises a buffer.

14. The pharmaceutical composition of claim 13, wherein said buffer comprises citric acid, succinic acid, pyrophosphoric acid, phosphoric acid, citrate, succinate, pyrophosphate, or phosphate.

15. The pharmaceutical composition of any one of claims 1-14, wherein said pharmaceutical composition further comprises a pharmaceutically acceptable excipient.

16. The pharmaceutical composition of any one of claims 1-15, wherein said pharmaceutical composition is substantially free of oxygen.

17. The pharmaceutical composition of any one of claims 1-16, wherein said pharmaceutical composition is supersaturated in LD.

18. The pharmaceutical composition of any one of claims 1-17, wherein the solubility of LD in said pharmaceutical composition is at least 5 g per liter at about 25° C.

19. The pharmaceutical composition of claim 18, wherein the solubility of LD in said pharmaceutical composition is at least 10 g per liter at about 25° C.

20. The pharmaceutical composition of any one of claims 1-19, wherein less than 10% of the LD prodrug acid addition salt is hydrolyzed when said pharmaceutical composition is stored at 5±3° C. for a period of 6 months.

21. The pharmaceutical composition of claim 20, wherein said pharmaceutical composition remains substantially free of precipitated solid LD for at least 6 months when stored at about 4° C.

22. The pharmaceutical composition of claim 21, wherein said pharmaceutical composition remains substantially free of precipitated solid LD for at least 12 months when stored at about 4° C.

23. The pharmaceutical composition of claim 22, wherein said pharmaceutical composition remains substantially free of precipitated solid LD for at least 18 months when stored at about 4° C.

24. The pharmaceutical composition of claim 23, wherein said pharmaceutical composition remains substantially free of precipitated solid LD for at least 24 months when stored at about 4° C.

25. The pharmaceutical composition of any one of claims 1-19, wherein said pharmaceutical composition remains substantially free of precipitated solid LD for at least 3 months when stored at about 25° C.

26. The pharmaceutical composition of claim 25, wherein said pharmaceutical composition remains substantially free of precipitated solid LD for at least 6 months when stored at about 25° C.

27. The pharmaceutical composition of claim 26, wherein said pharmaceutical composition remains substantially free of precipitated solid LD for at least 12 months when stored at about 25° C.

28. The pharmaceutical composition of claim 27, wherein said pharmaceutical composition remains substantially free of precipitated solid LD for at least 18 months when stored at about 25° C.

29. The pharmaceutical composition of any one of claims 1-19, wherein said pharmaceutical composition remains substantially free of precipitated solid LD for at least 48 hours when stored at about 25° C.

30. The pharmaceutical composition of any one of claims 1-19, wherein said pharmaceutical composition remains substantially free of precipitated solid LD for at least 24 hours when stored at about 37° C.

31. The pharmaceutical composition of any one of claims 1-19, wherein said pharmaceutical composition remains substantially free of precipitated solid LD when thawed after being stored frozen for at least 3 months.

32. The pharmaceutical composition of claim 31, wherein said pharmaceutical composition remains substantially free of precipitated solid LD when thawed after being stored frozen for at least 12 months.

33. A container comprising a pharmaceutical composition of any one of claims 1 to 32.

34. The container of claim 33, wherein the container is substantially impermeable to oxygen, said container comprising an atmosphere substantially free of oxygen.

35. The container of any one of claims 33 and 34, wherein the container is a drug reservoir of an ambulatory infusion pump.

36. A kit comprising (i) a pharmaceutical composition of any of claims 1 to 32; and (ii) instructions for administering the composition to a subject for the treatment of Parkinson's disease.

37. An ambulatory infusion pump system for the treatment of Parkinson's disease comprising:
(i) the pharmaceutical composition of any of claims 1-32 in a drug reservoir; and
(ii) at least one cannula or needle in fluid communication with the drug reservoir for subcutaneously infusing said pharmaceutical composition into a subject.

38. The system of claim 37 comprising at least two cannulas or needles.

39. The system of claim 38 comprising at least three cannulas or needles.

40. The system of claim 39 comprising at least four cannulas or needles.

41. The ambulatory infusion pump system of claim 37, wherein said pump system is a patch pump comprising an adhesive for adherence of the patch pump directly or indirectly to the skin of a subject.

42. The ambulatory infusion pump system of claim 37, further comprising software, memory, a data processing unit, and information input/output capability,
wherein the system is able to input, store and recall data comprising one or more of the subject's symptoms or drug responses related to Parkinson's disease, such symptoms selected from the group of tremor, hyperkinesia, dystonia, akinesia, bradykinesia, tremor, turning on, turning off, delayed time to on, and response failure.

43. The ambulatory infusion pump system of claim 37, further comprising software, memory, a data processing unit, and user input capability to input into the system information related to the ingestion of a meal, and the system thereafter adjusts the rate of infusion of the pharmaceutical composition.

44. The ambulatory infusion pump system of claim 43, wherein said pump system is programmed to increase the rate of infusion after a meal comprising protein.

45. The ambulatory infusion pump system of claim 37, further comprising software, memory, a data processing unit, and information input/output capability,
wherein the system is able to automatically increase the rate of infusion of the pharmaceutical composition, by a factor of two or more, at a preset time in the morning or after a period of at least four hours.

46. The ambulatory infusion pump system of claim 37, further comprising a data processing unit; and a motion sensor electrically connected to, or in RF communication with, the data processing unit to detect movement of the subject,
wherein the system recommends a change in the infusion rate in response to the data from the motion sensor.

47. A method for using the pharmaceutical composition of any of claims 1-32, said method comprising the step of visually inspecting the composition prior to use to determine whether said pharmaceutical composition is suitable for infusion into a subject, wherein a transparent pharmaceutical composition is suitable for infusion and a colored, or light scattering, or opaque pharmaceutical composition is not suitable for infusion.

48. The method of claim 47, wherein said pharmaceutical composition is packed in a kit or container that is configured to permit visual inspection of the pharmaceutical composition.

49. A method for treating Parkinson's disease in a subject, said method comprising subcutaneously infusing into said subject a pharmaceutical composition of any of claims 1-32 in an amount sufficient to treat Parkinson's disease.

50. The method of claim 49, wherein the administration regimen comprises a continuous infusion regimen.

51. The method of claim 49, wherein the administration regimen comprises an intermittent infusion regimen.

52. The method of claim 49, wherein the flow rate at an infused site is between 0.1 mL per hour and 2.5 mL per hour.

53. The method of claim 49, wherein the pH is from 2.4 to 3.9 and the infusion is substantially painless.

54. The method of claim 52, wherein the flow rate is greater than 0.3 mL per hour.

55. The method of any one of claims 49-54, wherein less than one tenth ( 1/10^{th}) of the infused sites are swollen, inflamed, or hard 24 hours or more after the infusion.

56. The method of claim 49, wherein the average hourly rate of infusion of the LD-prodrug is greater than 100 micromoles per hour.

57. The method of claim 56, wherein the average hourly infusion rate is greater than 200 micromoles per hour.

58. The method of claim 57, wherein the average hourly infusion rate is greater than 500 micromoles per hour.

59. The method of claim 49, wherein said method alleviates a motor or non-motor complication in a subject afflicted with Parkinson's disease.

60. The method of claim 59, wherein said motor or non-motor complication comprises tremor.

61. The method of claim 59, wherein said motor or non-motor complication comprises akinesia.

62. The method of claim 59, wherein said motor or non-motor complication comprises bradykinesia.

63. The method of claim 59, wherein said motor or non-motor complication comprises dyskinesia.

64. The method of claim 59, wherein said motor or non-motor complication comprises dystonia.

65. The method of claim 59, wherein said motor or non-motor complication comprises cognitive impairment.

66. The method of claim 59, wherein said motor or non-motor complication comprises disordered sleep.

67. The method of claim 59, further comprising the administration of an effective amount of carbidopa or carbidopa prodrug, or benserazide or a benserazide prodrug.

68. The method of claim 67, wherein said carbidopa or carbidopa prodrug, or benserazide or a benserazide prodrug, is administered orally or by infusion.

69. The method of claim 49, wherein hyaluronidase is coinfused with said pharmaceutical composition or pre-infused prior to said pharmaceutical composition.

70. The method of claim 49, comprising subcutaneous infusion of the pharmaceutical composition at two, three, four or greater than four infusion sites during a period of less than or equal to 24 hours.

71. The method of claim 70, comprising subcutaneous infusion of the LD prodrug pharmaceutical composition at two, three, four or greater than four infusion sites during a period of less than or equal to 24 hours using a multifurcated infusion set.

72. The method of claim 71, wherein the multifurcated infusion set is bifurcated, trifurcated or quadrifurcated.

73. A method of any of claims 70-72, comprising subcutaneously infusing the LD prodrug pharmaceutical composition for a period of 8 hours or more.

74. The method of any one of claims 49-73, comprising subcutaneously infusing into the subject an LD prodrug pharmaceutical composition at such a rate that:
(i) a circulating plasma LD concentration greater than 400 ng/mL is continuously maintained for a period of at least 8 hours during said infusion; and
(ii) at 60 minutes after the end of the infusion the plasma LD concentration is not greater than it was at the end of the infusion.

75. The method of claim 74, wherein 45 minutes after the end of the infusion the plasma LD concentration is not greater than it was at the end of the infusion.

76. The method of claim 74, wherein 30 minutes after the end of the infusion the plasma LD concentration is not greater than it was at the end of the infusion.

77. The method of any of claims 74-76, wherein the circulating plasma concentration of said LD prodrug during said infusion does not exceed 100 ng/mL.

78. The method of claim 77, wherein said LD prodrug pharmaceutical composition is subcutaneously infused at such a rate that the circulating plasma concentration of said LD prodrug during said infusion does not exceed 50 ng/mL.

79. The method of claim 78, wherein said LD prodrug pharmaceutical composition is subcutaneously infused at such a rate that circulating plasma concentration of said LD prodrug during said infusion does not exceed 30 ng/mL.

80. The method of claim 79, wherein said LD prodrug pharmaceutical composition is subcutaneously infused at such a rate that circulating plasma concentration of said LD prodrug during said infusion does not exceed 15 ng/mL.

81. The method of claim 74 where the average circulating plasma concentration of the LD prodrug is less than 1/500th of the average circulating plasma concentration of L-DOPA.

82. The method of any of claims 74-81, wherein said LD prodrug pharmaceutical composition is subcutaneously infused at such a rate that a circulating plasma LD concentration greater than 800 ng/mL is continuously maintained for a period of at least 8 hours during said infusion.

83. The method of claim 82, wherein said LD prodrug pharmaceutical composition is subcutaneously infused at such a rate that a circulating plasma LD concentration greater than 1,200 ng/mL is continuously maintained for a period of at least 8 hours during said infusion.

84. The method of claim 83, wherein said LD prodrug pharmaceutical composition is subcutaneously infused at such a rate that a circulating plasma LD concentration greater than 1,600 ng/mL is continuously maintained for a period of at least 8 hours during said infusion.

85. The method of any of claims 74-84, wherein said LD prodrug pharmaceutical composition is subcutaneously infused at such a rate that a circulating plasma LD concentration greater than 400 ng/mL is achieved within 60 minutes of the initiation of said infusion.

86. The method of claim 85, wherein said LD prodrug pharmaceutical composition is subcutaneously infused at such a rate that a circulating plasma LD concentration greater than 800 ng/mL is achieved within 60 minutes of the initiation of the infusion.

87. The method of claim 86, wherein said LD prodrug pharmaceutical composition is subcutaneously infused at such a rate that a circulating plasma LD concentration greater than 1,200 ng/mL is achieved within 60 minutes of the initiation of the infusion.

88. The method of claim 87, wherein said LD prodrug pharmaceutical composition is subcutaneously infused at such a rate that a circulating plasma LD concentration greater than 1,600 ng/mL is achieved within 60 minutes of the initiation of the infusion.

89. The method of any of claims 74-88, wherein said LD prodrug pharmaceutical composition is subcutaneously infused at such a rate that a circulating plasma LD concentration less than 7,500 ng/mL is continuously maintained for a period of at least 8 hours during said infusion.

90. The method of claim 89, wherein said LD prodrug pharmaceutical composition is subcutaneously infused at such a rate that a circulating plasma LD concentration less than 5,000 ng/mL is continuously maintained for a period of at least 8 hours during said infusion.

91. The method of claim 90, wherein said LD prodrug pharmaceutical composition is subcutaneously infused at such a rate that a circulating plasma LD concentration less than 2,500 ng/mL is continuously maintained for a period of at least 8 hours during said infusion.

92. The method of claim 91, wherein said LD prodrug pharmaceutical composition is subcutaneously infused at such a rate that a circulating plasma LD concentration less than 2,000 ng/mL is continuously maintained for a period of at least 8 hours during said infusion.

93. The method of any of claims 74-89, wherein the subject receives an average daily dose of less than 20 mL of said LD prodrug pharmaceutical composition.

94. The method of claim 93, wherein the average daily dose is greater than 5 mL

95. The method of any of claims 74-94, wherein during said infusion the circulating LD plasma concentration varies by less than +/−20% from its mean for a period of at least 1 hour.

96. The method of claim 95, wherein during said infusion the circulating LD plasma concentration varies by less than +/−10% from its mean for a period of at least 1 hour.

97. The method of claim 49, further comprising administering to the subject LD, or a prodrug of LD, via a route of administration other than subcutaneous infusion.

98. The method of claim 97, further comprising orally administering to the subject LD or a prodrug of LD.

99. The method of claim 98, wherein 50-100 mg of LD is orally administered to the patient within one hour before or after initiating an infusion of the LD prodrug pharmaceutical composition.

100. The method of claim 98, wherein 100-200 mg of LD is orally administered to the patient within one hour before or after initiating an infusion of the LD prodrug pharmaceutical composition.

101. The method of claim 98, wherein 200-300 mg of LD is orally administered to the patient within one hour before or after initiating an infusion of the LD prodrug pharmaceutical composition.

102. The method of claim 98, wherein greater than 300 mg of LD is orally administered to the patient within one hour before or after initiating an infusion of the LD prodrug pharmaceutical composition.

103. The method of claim 98, wherein:
(i) doses of at least 50 mg of LD are orally administered to the patient at three or more times during the day, each dose being separated from a previous dose by at least 2 hours; and
(ii) the total dose of oral LD administered during a 24 hour period is less than three times the molar dose of the infused LD prodrug pharmaceutical composition during said 24 hour period.

104. The method of claim 98, wherein:
(i) doses of at least 100 mg of LD are orally administered to the patient at three or more times during the day, each dose being separated from a previous dose by at least 2 hours; and
(ii) the total dose of oral LD administered during a 24 hour period is less than the molar dose of the infused LD prodrug pharmaceutical composition during said 24 hour period.

105. The method of claim 97, further comprising administering to the subject LD, or a prodrug of LD, via pulmonary delivery.

106. The method of claim 105, wherein 25-50 mg of LD is administered to the patient via pulmonary delivery within one hour before or after initiating an infusion of the LD prodrug pharmaceutical composition.

107. The method of claim 105, wherein 50-100 mg of LD is administered to the patient via pulmonary delivery within one hour before or after initiating an infusion of the LD prodrug pharmaceutical composition.

108. The method of claim 105, wherein 100-200 mg of LD is administered to the patient via pulmonary delivery within one hour before or after initiating an infusion of the LD prodrug pharmaceutical composition.

109. The method of claim 105, wherein 200-300 mg of LD is administered to the patient via pulmonary delivery within one hour before or after initiating an infusion of the LD prodrug pharmaceutical composition.

110. The method of claim 105, wherein:
(i) doses of at least 50 mg of LD are administered to the patient via pulmonary delivery at three or more times during the day, each dose being separated from a previous dose by at least 2 hours; and
(ii) the total dose of LD administered via pulmonary delivery during a 24 hour period is less than three times the molar dose of the infused LD prodrug pharmaceutical composition during said 24 hour period.

111. The method of claim 105, wherein:
(i) doses of at least 100 mg of LD are administered to the patient via pulmonary delivery at three or more times during the day, each dose being separated from a previous dose by at least 2 hours; and
(ii) the total dose of LD administered via pulmonary delivery during a 24 hour period is less than the molar dose of the infused LD prodrug pharmaceutical composition during said 24 hour period.

112. The method of claim 49, wherein the average daily molar amount of infused LD prodrug acid addition salt is less than 1.6 times the average daily molar amount of oral LD taken by the patient when not infusing the LD prodrug acid addition salt.

113. The method of claim 112, wherein the average daily molar amount of infused LD prodrug acid addition salt is less than 1.2 times the average daily molar amount of oral LD taken by the patient when not infusing the LD prodrug acid addition salt.

114. The method of claim 113, wherein the average daily molar amount of infused LD prodrug acid addition salt is less than 1.0 times the average daily molar amount of oral LD taken by the patient when not infusing the LD prodrug acid addition salt.

115. The method of claim 114, wherein the average daily molar amount of infused LD prodrug acid addition salt is less than 0.8 times the average daily molar amount of oral LD taken by the patient when not infusing the LD prodrug acid addition salt.

116. The method of any of claims 49-115, wherein said LD prodrug acid addition salt is an acid addition salt of LDEE or LDME.

117. The method of any of claims 49-116, wherein said LD prodrug pharmaceutical composition is subcutaneously infused into the subject via one or more ambulatory infusion pumps.

118. The method of any of claims 49-117, wherein the average hourly rate of infusion of the LD-prodrug is greater than 100 micromoles per hour.

119. The method of claim 118, wherein the average hourly infusion rate is greater than 200 micromoles per hour.

120. The method of claim 119, wherein the average hourly infusion rate is greater than 500 micromoles per hour.

121. The method of any one of claims 49-120, wherein hyaluronidase is coinfused with said pharmaceutical composition or pre-infused prior to said pharmaceutical composition.

122. The method of any of claims 49-120, comprising subcutaneous infusion of the pharmaceutical composition at two, three, four or greater than four infusion sites during a period of less than or equal to 24 hours.

123. The method of claim 122 where the flow rate of the infused solution at any site is between 0.1 mL per hour and 1 mL per hour.

124. The method of claim 123, comprising subcutaneous infusion of the LD prodrug pharmaceutical composition at two, three, four or greater than four infusion sites during a period of less than or equal to 24 hours using a multifurcated infusion set.

125. The method of claim 124, wherein the multifurcated infusion set is bifurcated, trifurcated or quadrifurcated.

126. The method of any of claims 49-125, wherein said LD prodrug acid addition salt is subcutaneously infused into said subject at one or more infusion sites, wherein the infusion volume at each of said infusion sites is less than 20 mL over a 24 hour period and the amount of LD prodrug acid addition salt administered at each of said infusion sites is less than 10 millimoles over a 24 hour period.

127. A method of manufacturing the pharmaceutical composition of any of claims 1-32, comprising dissolving dry crystallites of an LD prodrug acid addition salt or its free base in an aqueous solution.

128. A pharmaceutical composition comprising an aqueous solution containing (i) from 0.15 to 1.6 M LD prodrug acid addition salt, (ii) greater than 0.05 M carbidopa prodrug salt or benserazide salt, and (iii) having a pH of from 2.1 to 3.9, wherein said pharmaceutical composition is subcutaneously infusible.

129. A pharmaceutical composition comprising an aqueous solution containing from 0.15 to 1.6 M LD prodrug acid addition salt, and having a pH of from 2.1 to 3.9, wherein said pharmaceutical composition is subcutaneously infusible, and wherein said pharmaceutical composition remains substantially free of LD precipitate for at least 24 hours when stored at about 37° C.

130. A kit comprising:
(i) a first container comprising a sterile aqueous solution;
(ii) a second container comprising a sterile, dry, reconstitutable solid; and
(iii) instructions for combining the contents of the first container with the contents of the second container to form a pharmaceutical composition suitable for subcutaneous infusion into a subject and for infusing said pharmaceutical composition into a subject for the treatment of Parkinson's disease;
wherein said solid fully dissolves in said solution in less than 5 minutes at 25° C.; said infusible pharmaceutical composition comprises LDEE and has a pH of from 2.1 to 3.9; and less than 10% of the LDEE is hydrolyzed when said first container and said second container are stored at 5±3° C. for a period of 3 months.

131. The kit of claim 130, wherein subsequent to storage of said first container and said second container at 5±3° C. for a period of 3 months and then forming the infusible pharmaceutical composition, said infusible pharmaceutical composition remains substantially free of precipitated LD when kept at about 37° C. for at least 24 hours.

132. The kit of claim 130, wherein said sterile, dry, reconstitutable solid comprises LDEE.

133. A method for treating Parkinson's disease in a subject, said method comprising subcutaneously infusing into the subject a pharmaceutical composition comprising LDEE in an amount sufficient to treat said Parkinson's disease, wherein said pharmaceutical composition has a pH of 3.1±0.8 and comprises from 0.15 M to 1.6 M LDEE.

134. A subcutaneously infused aqueous pharmaceutical composition comprising a therapeutic agent and having a pH of from 2.4 to 3.0
i. infused at a rate greater than 0.01 mL per hour per infused site;
ii. with fewer than 1/10^{th} of the infused sites inflamed, swollen or hard 24 hours or more after the infusion.

135. The composition of claim 134, wherein the rate is greater than 0.1 mL per hour per infused site.

136. The composition of claim 135, wherein the rate is greater than 0.3 mL per hour per infused site.

137. The composition of any of claims 134-136, wherein the infusion is substantially painless.

138. The composition of any of claims 134-137, wherein the therapeutic agent alleviates a symptom of PD.

139. A method for subcutaneously infusing a pharmaceutical composition comprising the steps of:
(i) providing a subcutaneously infusible, aqueous pharmaceutical composition containing 0.15 M-1.6 M LD prodrug acid addition salt and a pH of from 2.1 to 3.9, wherein less than 10% of the LD prodrug acid addition salt is hydrolyzed when said pharmaceutical composition is stored at 5±3° C. for a period of 6 months; and
(ii) inserting the infusible pharmaceutical composition into an infusion pump,
wherein said pharmaceutical composition remains substantially free of precipitated LD when kept at about 25° C. for at least 24 hours.

140. The method of claim 139, wherein said infusible pharmaceutical composition comprises a pharmaceutical composition of any of claims 1-32.

141. The method of claim 49, further comprising subcutaneously infusing into the subject said pharmaceutical composition in a pulsed dosing regimen, wherein said pulsed dosing regimen comprises (i) a delivery period during which said LD prodrug solution is infused at a first site for from 1 second to 3 hours; and (ii) following step (i), a non-delivery period during which said LD prodrug solution is administered at a substantially reduced rate at said first site for from 10 to 120 minutes, and repeating steps (i) and (ii).

142. A method for treating Parkinson's disease in a subject, said method comprising:
(i) subcutaneously infusing into said subject a LD prodrug acid addition salt; and
(ii) delivering LD, or a prodrug of LD, via a second route of administration other than subcutaneous infusion,
wherein (a) 50-500 mg of LD, or a prodrug of LD, is administered to the patient via said second route of administration within one hour before or after initiating an infusion of the LD prodrug pharmaceutical composition; and (b) a circulating plasma LD concentration less than 5,000 ng/mL is continuously maintained for a period of at least 8 hours during said infusion.

143. The method of claim 142, wherein 50-100 mg of LD, or a prodrug of LD, is administered to the patient via said second route of administration within one hour before or after initiating an infusion of the LD prodrug pharmaceutical composition.

144. The method of claim 142, wherein 100-200 mg of LD, or a prodrug of LD, is administered to the patient via said second route of administration within one hour before or after initiating an infusion of the LD prodrug pharmaceutical composition.

145. The method of claim 142, wherein 200-300 mg of LD, or a prodrug of LD, is administered to the patient via said second route of administration within one hour before or after initiating an infusion of the LD prodrug pharmaceutical composition.

146. The method of claim 142, wherein 300-500 mg of LD, or a prodrug of LD, is administered to the patient via said second route of administration within one hour before or after initiating an infusion of the LD prodrug pharmaceutical composition.

147. A method for treating Parkinson's disease in a subject, said method comprising:
(i) subcutaneously infusing into said subject a LD prodrug acid addition salt; and
(ii) delivering LD, or a prodrug of LD, via a second route of administration other than subcutaneous infusion;
wherein (a) doses of 50-500 mg of LD, or a prodrug of LD, are administered to the patient via said second route of administration at three or more times during the day, each dose being separated from a previous dose by at least 2 hours; and (b) the total dose of LD, or a prodrug of LD, administered to the patient via said second route of administration during a 24 hour period is less than three times the molar dose of the infused LD prodrug acid addition salt during said 24 hour period.

148. The method of claim 147, wherein:
(a) doses of at least 100-400 mg of LD, or a prodrug of LD, are administered to the patient via said second route of administration at three or more times during the day, each dose being separated from a previous dose by at least 2 hours; and
(b) the total dose of LD, or a prodrug of LD, administered to the patient via said second route of administration during a 24 hour period is less than the molar dose of the infused LD prodrug acid addition salt during said 24 hour period.

149. The method of any of claims 142-148, wherein said second route of administration is oral administration.

150. The method of any of claims 142-148, wherein said second route of administration is pulmonary administration.

151. A method for treating Parkinson's disease in a subject, said method comprising subcutaneously infusing into the subject a LD prodrug solution in a pulsed dosing regimen, wherein said pulsed dosing regimen comprises (i) a delivery period during which said LD prodrug solution is infused at a first site for from 1 second to 3 hours; and (ii) following step (i), a non-delivery period during which said LD prodrug solution is administered at a substantially reduced rate at said first site for from 10 to 120 minutes, and repeating steps (i) and (ii).

152. The method of claim 151, wherein said delivery period is repeated at least twice over an 8 hour period.

153. A method for treating Parkinson's disease in a subject, said method comprising subcutaneously infusing into the subject a LD prodrug solution in a pulsed dosing regimen, wherein said pulsed dosing regimen comprises (i) a delivery period during which said LD prodrug solution is infused at a first site for from 1 second to 3 hours; and (ii) following step (i), a non-delivery period during which said LD prodrug solution is administered at a substantially reduced rate at said first site for from 10 to 120 minutes; (iii) a delivery period during which said LD prodrug solution is infused at a second site for from 1 second to 3 hours; and (iv) following step (iii), a non-delivery period during which said LD prodrug solution is infused at a substantially reduced rate to said second site for from 10 to 120 minutes, and optionally repeating steps (i), (ii), (iii), and (iv).

154. The method of claim 153, wherein said pulsed dosing regimen further comprises (v) a delivery period during which said LD prodrug solution is infused at a third site for from 1 second to 3 hours; and (vi) following step (v), a non-delivery period during which said LD prodrug solution is administered at a substantially reduced rate to said third site for from 10 to 120 minutes, and optionally repeating steps (v) and (vi).

155. The method of claim 154, wherein said pulsed dosing regimen further comprises (vii) a delivery period during which said LD prodrug solution is infused at a fourth site for from 1 second to 3 hours; and (viii) following step (vii), a non-delivery period during which said LD prodrug solution is administered at a substantially reduced rate to said fourth site for from 10 to 120 minutes, and optionally repeating steps (vii) and (viii).

156. The method of claim 155, wherein said pulsed dosing regimen further comprises (ix) a delivery period during which said LD prodrug solution is infused at a fifth site for from 1 second to 3 hours; and (x) following step (ix), a non-delivery period during which said LD prodrug solution is administered at a substantially reduced rate to said fifth site for from 10 to 120 minutes, and optionally repeating steps (ix) and (x).

157. The method of any of claims 153-156, wherein said delivery period is repeated at least twice over an 8 hour period.

158. The method of claim 157, wherein said delivery period is repeated every 60 to 120 minutes over an 8 hour period.

159. The method of claim 157, wherein said pulsed dosing regimen comprises administration of said LD prodrug solution to a plurality of sites sequentially, wherein each of said sites are separated from each other by at least 1 cm.

160. The method of claim 159, wherein said pulsed dosing regimen comprises administration of said LD prodrug solution to a plurality of sites sequentially, wherein each of said sites are separated from each other by at least 3 cm.

161. The method of claim 160, wherein said pulsed dosing regimen comprises administration of said LD prodrug solution to a plurality of sites sequentially, wherein each of said sites are separated from each other by at least 5 cm.

162. The methods of any of claims 151-161, wherein the time averaged rate at which said LD prodrug is administered during said non-delivery period is less than 10% of the time averaged rate at which said LD prodrug is infused during said delivery period.

163. The method of claim 162, wherein the time averaged rate at which said LD prodrug is administered during said non-delivery period is from 0 μmol/minute to 0.25 μmol/minute.

164. The method of claim 162, wherein the time averaged rate at which said LD prodrug is administered during said non-delivery period is from 0.25 μmol/minute to 0.75 μmol/minute.

165. The methods of any of claims 151-164, wherein the non-delivery period is at least twice as long as said delivery period.

166. The method of any of claims 151-165, wherein (a) a circulating plasma LD concentration greater than 400 ng/mL and less than 7,500 ng/mL is continuously maintained in said subject for a period of at least 8 hours during said pulsed dosing regimen.

167. The method of claim 166, wherein at least ¼ of the total daily molar dosage of the LD prodrug and of LD is by subcutaneous infusion of the LD-prodrug; or wherein at least ½ of the total daily molar dosage of the LD prodrug and of LD is by subcutaneous infusion of the LD-prodrug; or wherein at least ¾ of the total daily molar dosage of the LD prodrug and of LD is by subcutaneous infusion of the LD-prodrug.

168. The method of claim 167, wherein the circulating LD plasma concentration varies by less than +/−20% from its mean for a period of at least 8 hours during said pulsed dosing regimen.

169. The method of claim 167, wherein the sum of said LD prodrug administered over all sites over a 24 hour period is less than 15 millimoles and the sum of infusion volume administered over all sites over a 24 hour period is less than 40 mL; or wherein the sum of said LD prodrug administered over all sites over a 24 hour period is less than 10 millimoles and the sum of infusion volume administered over all sites over a 24 hour period is less than 25 mL.

170. The method of claim 169, wherein the sum of said LD prodrug administered over all sites over a 24 hour period is from 1.0 and 15 millimoles and the sum of infusion volume administered over all sites over a 24 hour period is between 3 and 40 mL over a 24 hour period.

171. The method of claim 170, wherein the sum of said LD prodrug administered over all sites over a 24 hour period is from 1.0 and 10 millimoles and the sum of infusion volume administered over all sites over a 24 hour period is between 3 and 16 mL over a 24 hour period.

172. The method of any of claims 151-171, wherein an extracellular matrix degrading enzyme is administered at each of said sites.

173. The method of claim 172, wherein said extracellular matrix degrading enzyme is administered prior to administration of said LD prodrug.

174. The method of claim 172, wherein said extracellular matrix degrading enzyme is administered during said non-delivery period.

175. The method of claim 172, wherein said extracellular matrix degrading enzyme is co-infused with said LD prodrug solution.

176. The method of any one of claims 172-175, wherein said extracellular matrix degrading enzyme is a hyaluronidase.

177. The method of any one of claims 151-176, wherein said LD prodrug solution is administered at a depth between 5 mm and 15 mm below the surface of the skin of said subject.

178. The method of any one of claims 151-176, wherein said LD prodrug solution is administered into subcutis or fat at a depth between 2 mm and 10 mm below the dermis of said subject.

179. The method of any one of claims 151-176, wherein said LD prodrug solution comprises greater than 0.15 M LD prodrug and is substantially free of precipitated solid LD when stored for 48 hours at about 25° C.

180. The method of any one of claims 151-176, wherein said LD prodrug solution comprises a greater than 0.15 M LD prodrug and is substantially free of precipitated solid LD when stored for 3 months at about 5±3° C. and when subsequently stored for 16 hours at 37° C.

181. The method of any one of claims 151-176, wherein said LD prodrug solution remains substantially free of precipitated solid LD when thawed after being stored frozen for at least 3 months.

182. The method of any one of claims 151-181, wherein said LD prodrug is selected from LDAs, LDEs, and salts thereof.

183. The method of claim 182, wherein said LD prodrug is LDEE, LDME, or a salt thereof.

184. The method of claim 183, wherein said LD prodrug solution has a pH of from 2.5 to 4.6 and comprises from 0.15 M to 1.6 M LDEE or LDME.

185. The method of claim 184, wherein said LD prodrug solution has a pH of from 2.5 to 4.6 and comprises from 0.25 M to 0.75 M LDEE or LDME.

186. The method of claim 185, wherein said LD prodrug solution has a pH of from 2.6 to 3.9 and comprises from 0.25 M to 0.75 M LDEE or LDME.

187. The method of claim 186, wherein said LD prodrug solution has a pH of from 3.0-6.0 and comprises from 0.15 M to 4.0 M LDEE, or a salt thereof.

188. The method of claim 187, wherein said LD prodrug solution has a pH of from 3.0-5.5.

189. The method of claim 187, wherein said LD prodrug solution has a pH of 3.7±0.3.

190. The method of claim 187, wherein said LD prodrug solution has a pH of 4.0±0.3.

191. The method of claim 187, wherein said LD prodrug solution has a pH of 4.5±0.3.

192. The method of claim 183, wherein said LD prodrug solution has a pH of from 2.1 to 3.9 and comprises from 0.15 M to 1.6 M LDEE, or a salt thereof.

193. The method of any of claims 183-192, wherein said LD prodrug solution comprises a buffer.

194. The method of any one of claims 151-193, wherein said LD prodrug solution is subcutaneously infused into the subject via one or more ambulatory infusion pumps.

195. The method of claim 194, wherein said LD prodrug solution is subcutaneously infused into the subject via two ambulatory infusion pumps.

196. The method of claim 194, wherein said one or more ambulatory infusion pumps comprise a two-compartment infusion pump.

197. The method of any one of claims 151-196, wherein said LD prodrug solution is subcutaneously infused into the subject via a bifurcated, trifurcated, or quadrifurcated infusion set.

198. The method of any of claims 194-197, further comprising the steps of:
(i) providing an aqueous solution comprising greater than 0.15 M LD prodrug and having a pH of from 1.5 to 2.5, wherein less than 10% of the LD prodrug is hydrolyzed when stored at 5±3° C. for a period of 6 months;
(ii) raising the pH of said solution to from 3.0 to 6.0 to form said LD prodrug solution and diluting the solution; and
(iii) infusing at least a portion of said LD prodrug solution into said subject.

199. The method of claim 198, comprising the step of providing a solution comprising greater than 0.15 M LD prodrug and having a pH of 2.7±0.5, and raising the pH of said solution to 4.0±0.8 to form said LD prodrug solution.

200. The method of claim 198, wherein said pH is adjusted with a salt of citric acid, pyrophosphoric acid, succinic acid, or phosphoric acid.

201. An ambulatory infusion pump system for the treatment of Parkinson's disease in a subject comprising:
(i) a drug reservoir comprising a LD prodrug solution;
(ii) a first cannula in fluid communication with the drug reservoir for subcutaneously administering said LD prodrug solution into said subject at a first site; and
(iii) a software unit comprising a program for controlled infusion of said LD prodrug solution in a pulsed dosing regimen, wherein said pulsed dosing regimen comprises (a) a delivery period during which said LD prodrug solution is administered to said first site for from 1 second to 3 hours; and (b) following step (a), a non-delivery period during which said LD prodrug solution is administered at a substantially reduced rate to said first site for from 10 to 120 minutes, and optionally repeating steps (a) and (b).

202. The ambulatory infusion pump system of claim 201, further comprising:
(iv) a second cannula in fluid communication with the drug reservoir for infusing said LD prodrug solution into said subject at a second site,
wherein said pulsed dosing regimen further comprises (c) a delivery period during which said LD prodrug solution is administered to said second site for from 1 second to 3 hours; and (d) following step (c), a non-delivery period during which said LD prodrug solution is administered at a substantially reduced rate to said second site for from 10 to 120 minutes, and optionally repeating steps (c) and (d).

203. The ambulatory infusion pump system of claim 202, further comprising:
(v) a third cannula in fluid communication with the drug reservoir for infusing said LD prodrug solution into said subject at a third site,
wherein said pulsed dosing regimen further comprises (e) a delivery period during which said LD prodrug solution is administered to said third site for from 1 second to 3 hours; and (f) following step (e), a non-delivery period during which said LD prodrug solution is administered at a substantially reduced rate to said third site for from 10 to 120 minutes, and optionally repeating steps (e) and (f).

204. The ambulatory infusion pump system of claim 203, further comprising:
(vi) a fourth cannula in fluid communication with the drug reservoir for infusing said LD prodrug solution into said subject at a fourth site,
wherein said pulsed dosing regimen further comprises (g) a delivery period during which said LD prodrug solution is administered to a fourth site for from 1 second to 3 hours; and (h) following step (g), a non-delivery period during which said LD prodrug solution is administered at a substantially reduced rate to said fourth site for from 10 to 120 minutes, and optionally repeating steps (g) and (h).

205. The ambulatory infusion pump system of claim 204, further comprising:
(vii) a fifth cannula in fluid communication with the drug reservoir for infusing said LD prodrug solution into said subject at a fifth site,
wherein said pulsed dosing regimen further comprises (i) a delivery period during which said LD prodrug solution is administered to a fifth site for from 1 second to 3 hours; and (j) following step (i), a non-delivery period during which said LD prodrug solution is administered at a substantially reduced rate to said fifth site for from 10 to 120 minutes, and optionally repeating steps (i) and (j).

206. The ambulatory infusion pump system of any of claims 201-205, wherein said ambulatory infusion pump system is programmed to repeat said delivery period at least twice over an 8 hour period.

207. The ambulatory infusion pump system of claim 206, wherein said ambulatory infusion pump system is programmed to repeat said delivery period every 60 to 120 minutes over an 8 hour period.

208. The ambulatory infusion pump system of any of claims 201-205, wherein said ambulatory infusion pump system is programmed to administer no LD prodrug during said non-delivery period.

209. The ambulatory infusion pump system of any of claims 201-205, wherein said ambulatory infusion pump system is programmed for a pulsed dosing regimen in which said non-delivery period is at least twice as long as said delivery period.

210. The ambulatory infusion pump system of any of claims 201-205, wherein said pulsed dosing regimen comprises administration of said LD prodrug solution to a plurality of sites sequentially, wherein each of said sites are separated from each other by at least 1 cm.

211. The ambulatory infusion pump system of claim 210, wherein said ambulatory infusion pump system comprises an adhered patch bearing a plurality of cannulas positioned at said plurality of sites.

212. The ambulatory infusion pump system of claim 211, wherein said adhered patch comprises two, three, four, five, or six cannulas.

213. A method for treating Parkinson's disease in a subject, said method comprising subcutaneously infusing into the subject an LDEE or LDME solution in an amount sufficient to treat said Parkinson's disease, wherein said LDEE or LDME solution has a pH of 3.3±0.6 and comprises from 0.25 M to 0.75 M LDEE or LDME.

214. The method of claim 213, wherein said LDEE or LDME solution is substantially free of precipitated solid LD when stored for 48 hours at about 25° C.

215. The method of claim 213, wherein said LDEE or LDME solution is substantially free of precipitated solid LD when stored for 3 months at about 5±3° C. and when subsequently stored for 16 hours at 37° C.

216. A method for treating Parkinson's disease in a subject, said method comprising subcutaneously administering into the subject an LDEE solution in an amount sufficient to treat said Parkinson's disease, wherein said LDEE solution has a pH of 3.7±0.3 and comprises from 0.15 M to 1.5 M LDEE, or a salt thereof.

217. The method of claim 216, wherein said LDEE solution is substantially free of precipitated solid LD when stored for 48 hours at about 25° C.

218. The method of claim 216, wherein said LDEE solution is substantially free of precipitated solid LD when stored for 3 months at about 5±3° C. and when subsequently stored for 16 hours at 37° C.

219. The method of claim 216, wherein said LDEE solution remains substantially free of precipitated solid LD when thawed after being stored frozen for at least 3 months.

220. The method of claim 216, wherein said LDEE solution is subcutaneously infused into the subject via one or more ambulatory infusion pumps.

221. The method of claim 216, wherein said LDEE solution is administered in a pulsed dosing regimen.

222. An LDEE or LDME solution having a pH of 3.3±0.6 and comprising from 0.25 M to 0.75 M LDEE, LDME, or a salt thereof.

223. An LDEE solution having a pH of 3.7±0.3 and comprising from 0.15 M to 1.5 M LDEE, or a salt thereof.

224. The LDEE solution of claim 223, wherein said solution further comprises a buffer.
